(19) United States
(12) Patent Application Publication
(10) Pub. No.: US 2022/0177490 A1
(43) Pub. Date: Jun. 9, 2022

(54) COMPOUNDS HAVING BOTH EFFECTS OF BET BROMODOMAIN PROTEIN INHIBITION AND PD-L1 GENE REGULATION

(71) Applicant: MEDSHINE DISCOVERY INC., Nanjing, Jiangsu (CN)

(72) Inventors: Chunli SHEN, Shanghai (CN); Yuchuan ZHU, Shanghai (CN); Ting WANG, Shanghai (CN); Chengde WU, Shanghai (CN); Shuhui CHEN, Shanghai (CN)

(73) Assignee: MEDSHINE DISCOVERY INC., Nanjing, Jiangsu (CN)

(21) Appl. No.: 17/436,821

(22) PCT Filed: Mar. 6, 2020
(86) PCT No.: PCT/CN2020/078187

§ 371 Date: Sep. 7, 2021

(30) Foreign Application Priority Data
Mar. 7, 2019 (CN) 201910172886.0

(51) Int. Cl. C07D 495/14; A61P 35/00; C07D 519/00

(52) U.S. Cl. CPC C07D 495/14; C07D 519/00; A61P 35/00

(57) ABSTRACT

Compounds having both BET bromodomain protein inhibitory activity and PD-L1 gene expression regulation, and use thereof in preparing medicaments for treating tumor diseases related to BET bromodomain protein inhibition and PD-L1 gene expression. Specifically disclosed are a compound as shown in formula (1), and an isomer and a pharmaceutically acceptable salt thereof.

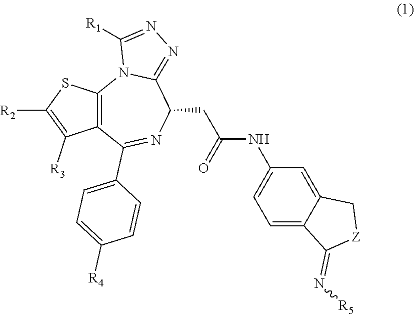

### REFERENCE TO RELATED APPLICATIONS

The present application is a United States National Phase under 35 U.S.C. § 371 of International Application No. PCT/CN2020/078187, filed Mar. 6, 2020, which claims the priority of CN201910172886.0 filed on Mar. 7, 2019. Both of the foregoing applications are incorporated by reference in their entirety for all purposes.

### FIELD OF THE INVENTION

The present disclosure relates to a class of compounds that simultaneously inhibit the activity of BET Bromodomain protein and regulate the expression of PD-L1 gene, and use thereof in the manufacture of a medicament for treating diseases related to BET bromodomain protein inhibition and PD-L1 gene expression. Specifically, the present disclosure relates to a compound as shown in formula (I) and a pharmaceutically acceptable salt thereof.

### BACKGROUND OF THE INVENTION

BET (bromodomain and extraterminal) protein BRD4 is bonded directly with acetylated lysine and other nucleoproteins on the tail of histone via RNA polymerase II (Pol II) to promote gene transcription. BET inhibitors have shown great potential in anti-tumor, and their anti-tumor activity has been confirmed in clinical trials for hematological malignancies. BET inhibitors also have the effect of regulating PD-L1 gene expression and enhancing cytotoxic T cell activity, thereby inhibiting tumor progression in ovarian cancer models.

### SUMMARY OF THE INVENTION

The present disclosure provides a compound as shown in formula (I), or an isomer or a pharmaceutically acceptable salt thereof,

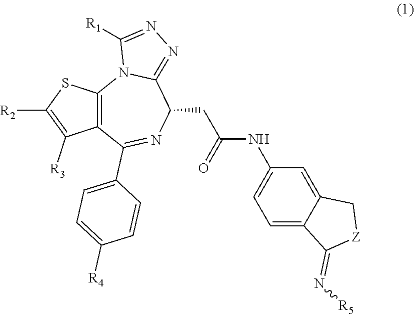

wherein:

R₁, R₂ and R₃ are each independently selected from C₁₋₃ alkyl optionally substituted with 1, 2 or 3 Rₐ;

R₄ is selected from the group consisting of H, F, C₁, Br, I, OH, and NH₂;

R₅ is selected from the group consisting of H, OH, NH₂, CN, C₁₋₆ alkyl, C₁₋₆ alkylamino, C₁₋₆ alkoxy and 4- to 6-membered heterocycloalkyl, wherein said C₁₋₆ alkyl, C₁₋₆ alkylamino, C₁₋₆ alkoxy or 4- to 6-membered heterocycloalkyl is each independently optionally substituted with 1, 2 or 3 R_{b};

Z is selected from the group consisting of O, NR₆ and CHR₆;

R₆ is selected from the group consisting of H and C₁₋₃ alkyl optionally substituted with 1, 2 or 3 R_{c};

alternatively, R₅, R₆ and the atoms to which they are attached together form a 5- to 6-membered heterocycloalkenyl or a 5- to 6-membered heteroaryl, wherein the 5- to 6-membered heterocycloalkenyl or 5- to 6-membered heteroaryl is each independently optionally substituted with 1, 2 or 3 R_{d};

Rₐ, R_{c}, and R_{d} are each independently selected from the group consisting of F, Cl, Br, I, OH, NH₂, and CH₃;

R_{b} is selected from the group consisting of F, Cl, Br, I, OH, NH₂, CN, C₁₋₃ alkyl and C₁₋₃ alkoxy, wherein the C₁₋₃ alkyl or C₁₋₃ alkoxy is each independently optionally substituted with 1, 2 or 3 R;

R is each independently selected from the group consisting of F, Cl, Br, I, OH, NH₂ and CH₃; and

the 4- to 6-membered heterocycloalkyl, 5- to 6-membered heterocycloalkenyl, and 5- to 6-membered heteroaryl each contains 1, 2, 3 or 4 heteroatoms or heteroatom groups independently selected from the group consisting of —NH—, —O—, —S— and N.

In some embodiments disclosed herein, the above-mentioned R₁, R₂ and R₃ are each independently selected from CH₃ optionally substituted with 1, 2 or 3 Rₐ, and other variables are as defined herein.

In some embodiments disclosed herein, the above-mentioned R₁, R₂, and R₃ are each independently selected from CH₃, and other variables are as defined herein.

In some embodiments disclosed herein, the above-mentioned R₄ is selected from Cl, and other variables are as defined herein.

In some embodiments disclosed herein, the above-mentioned R_{b} is selected from the group consisting of F, Cl, Br, I, OH, NH₂, CN, CH₃, CH₂CH₃, and OCH₃, wherein the CH₃, CH₂CH₃, and OCH₃ are optionally substituted with 1, 2, or 3 R, and other variables are as defined herein.

In some embodiments disclosed herein, the above-mentioned R_{b} is selected from the group consisting of F, Cl, Br, I, OH, NH₂, CN, CH₃, CH₂F, CHF₂, CF₃, CH₂CH₃, and OCH₃, and other variables are as defined herein.

In some embodiments disclosed herein, the above-mentioned R₅ is selected from the group consisting of H, OH, NH₂, CN, C₁₋₃ alkyl, C₁₋₃ alkylamino, C₁₋₃ alkoxy, and oxetanyl, wherein the C₁₋₃ alkyl, C₁₋₃ alkylamino, C₁₋₃ alkoxy, or oxetanyl are each independently optionally substituted with 1, 2, or 3 R_{b}, and other variables are as defined herein.

In some embodiments disclosed herein, the above-mentioned R₅ is selected from the group consisting of H, OH, NH₂, CN, CH₃, CH₂CH₃, CH(CH₃)₂, OCH₃, OCH₂CH₃, NH(CH₃) and oxetanyl, wherein the CH₃, CH₂CH₃, CH(CH₃)₂, OCH₃, OCH₂CH₃, NH(CH₃) or oxetanyl are each independently optionally substituted with 1, 2 or 3 R_{b}, and other variables are as defined herein.

In some embodiments disclosed herein, the above-mentioned R₅ is selected from the group consisting of H, OH, NH₂, CN, CH₃, CH₂F, CHF₂, CF₃, CH₂CH₃, CH(CH₃)₂, OCH₃, OCH₂F, OCHF₂, OCF₃, OCH₂CH₃, OCH(CH₃)₂, OCH₂CH₂OCH₃, NH(CH₃), N(CH₃)₂, NHCH(CH₃)₂, and

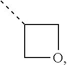

and other variables are as defined herein.

In some embodiments disclosed herein, the above-mentioned R₅, R₆ and the atoms to which they are attached together form 4,5-dihydroisoxazolyl, pyrazolyl, pyrrolyl or imidazolyl, wherein the 4,5-dihydroisoxazolyl, pyrazolyl, pyrrolyl or imidazolyl are each independently optionally substituted with 1, 2 or 3 R_{d}.

In some embodiments disclosed herein, the above-mentioned R₅, R₆ and the atoms to which they are attached together form

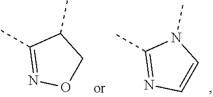

and other variables are as defined herein.

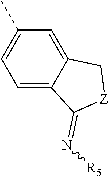

In some embodiments disclosed herein, the above-mentioned moiety R₅ is selected from the group consisting of

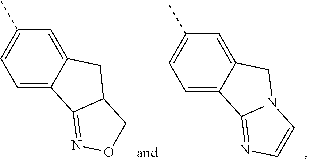

and other variables are as defined herein.

The present disclosure also includes some embodiments that are obtained by combining any of the above-mentioned variables.

In some embodiments disclosed herein, the above-mentioned compound, or an isomer or a pharmaceutically acceptable salt thereof is selected from the group consisting of

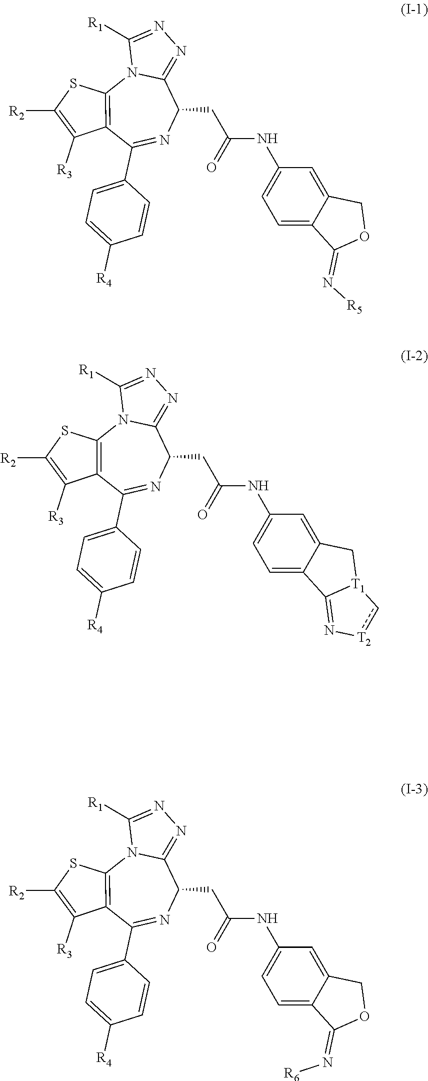

wherein, [symbol] is selected from the group consisting of single bond and double bond;

R₁, R₂, R₃, R₄ and R₅ are as defined herein;

T₁ is CH or N;

T₂ is CH, N, or O.

In some embodiments disclosed herein, the above-mentioned compound, or an isomer or a pharmaceutically acceptable salt thereof is selected from the group consisting of

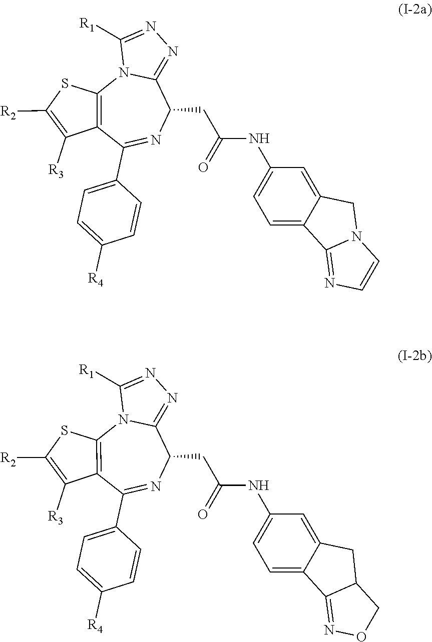

[symbol] is selected from the group consisting of single bond and double bond;

R₁, R₂, R₄ and R₃ are as define herein.

The present disclosure also provides compounds as shown in the formulae below, or isomers or pharmaceutically acceptable salts thereof, wherein the compounds are selected from the group consisting of

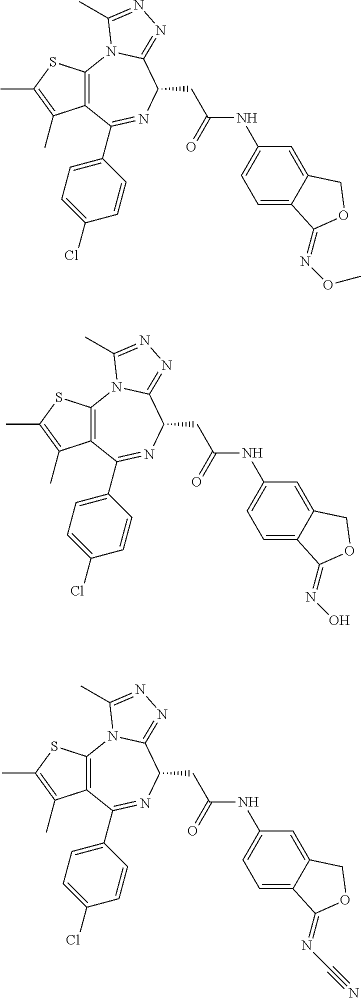

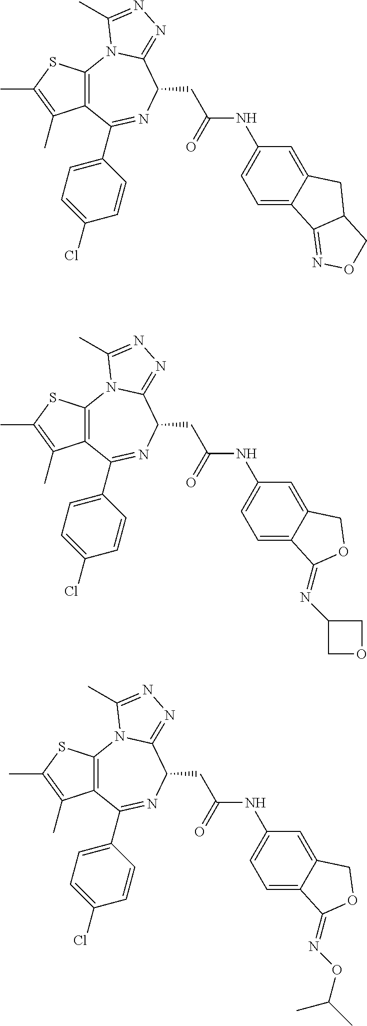 [SMILES: Cc1sc2c(c1C)C(c1ccc(Cl)cc1)=N[C@@H](CC(=O)Nc1ccc3c(c1)CC1CON=C31)c1nnc(C)n1-2 ; Cc1sc2c(c1C)C(c1ccc(Cl)cc1)=N[C@@H](CC(=O)Nc1ccc3c(c1)CO/C3=N\C1COC1)c1nnc(C)n1-2 ; Cc1sc2c(c1C)C(c1ccc(Cl)cc1)=N[C@@H](CC(=O)Nc1ccc3c(c1)CO/C3=N\OC(C)C)c1nnc(C)n1-2]

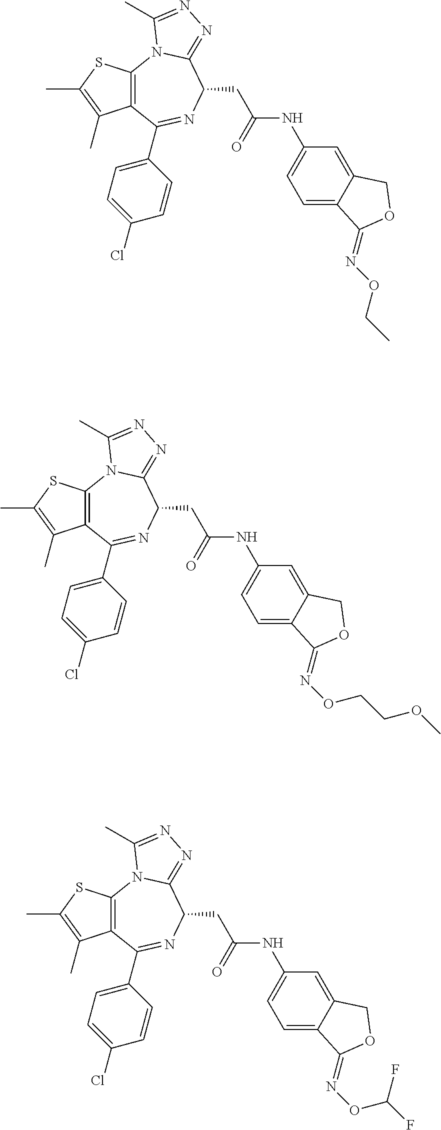

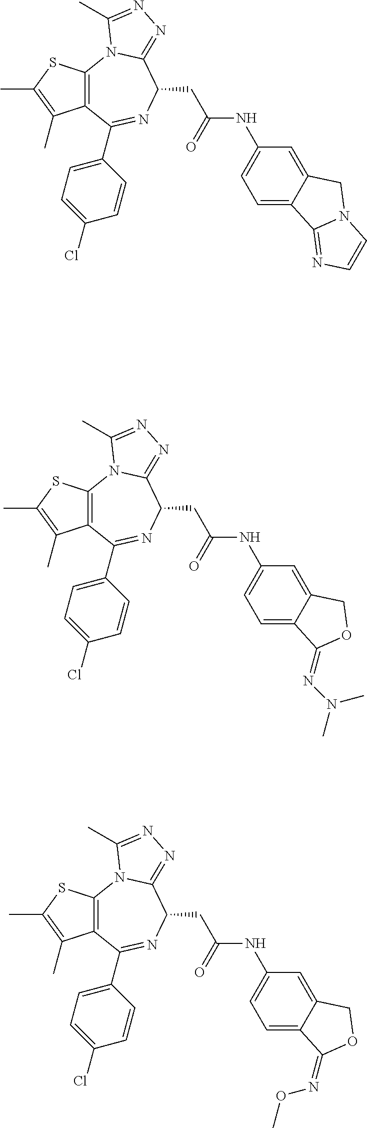

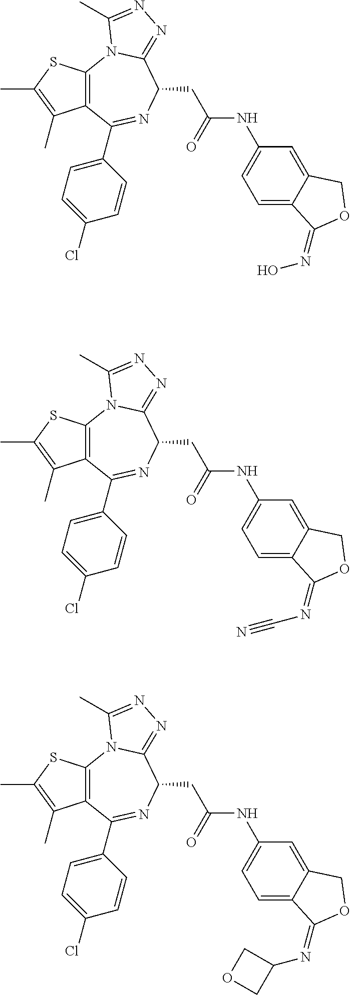 [SMILES: Cc1sc2c(c1C)C(c1ccc(Cl)cc1)=N[C@@H](CC(=O)Nc1ccc3c(c1)CO/C3=N/O)c1nnc(C)n1-2 ; Cc1sc2c(c1C)C(c1ccc(Cl)cc1)=N[C@@H](CC(=O)Nc1ccc3c(c1)CO/C3=N/C#N)c1nnc(C)n1-2 ; Cc1sc2c(c1C)C(c1ccc(Cl)cc1)=N[C@@H](CC(=O)Nc1ccc3c(c1)CO/C3=N/C1COC1)c1nnc(C)n1-2]

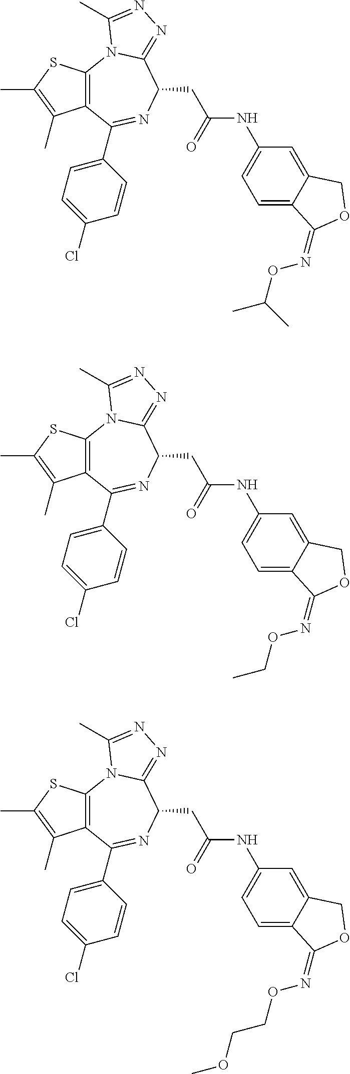 [SMILES: Cc1sc2c(c1C)C(c1ccc(Cl)cc1)=N[C@@H](CC(=O)Nc1ccc3c(c1)CO/C3=N/OC(C)C)c1nnc(C)n1-2 ; CCO/N=C1/OCc2cc(NC(=O)C[C@@H]3N=C(c4ccc(Cl)cc4)c4c(sc(C)c4C)-n4c(C)nnc43)ccc21 ; COCCO/N=C1/OCc2cc(NC(=O)C[C@@H]3N=C(c4ccc(Cl)cc4)c4c(sc(C)c4C)-n4c(C)nnc43)ccc21]

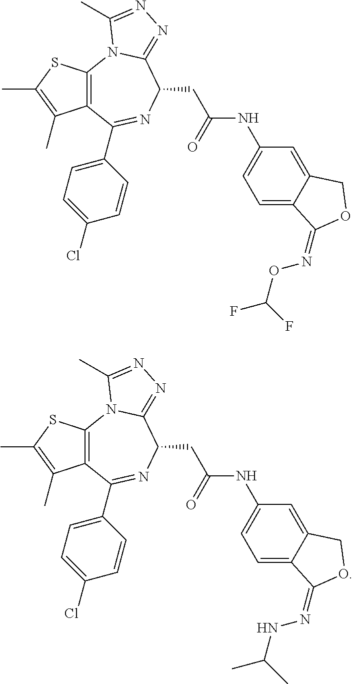 [SMILES: Cc1sc2c(c1C)C(c1ccc(Cl)cc1)=N[C@@H](CC(=O)Nc1ccc3c(c1)CO/C3=N/OC(F)F)c1nnc(C)n1-2 ; Cc1sc2c(c1C)C(c1ccc(Cl)cc1)=N[C@@H](CC(=O)Nc1ccc3c(c1)CO/C3=N/NC(C)C)c1nnc(C)n1-2]

In some embodiments disclosed herein, the above-mentioned compound, or an isomer or a pharmaceutically acceptable salt thereof is selected from the group consisting of

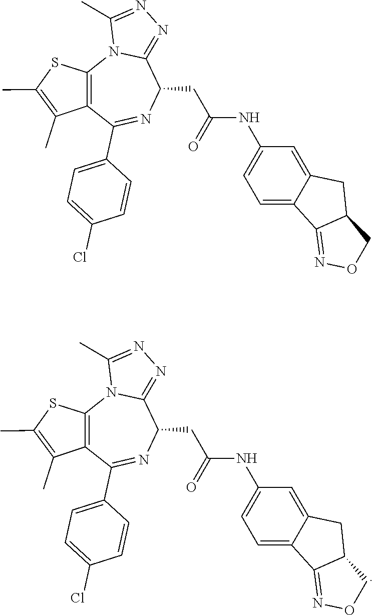 [SMILES: Cc1sc2c(c1C)C(c1ccc(Cl)cc1)=N[C@@H](CC(=O)Nc1ccc3c(c1)C[C@H]1CON=C31)c1nnc(C)n1-2 ; Cc1sc2c(c1C)C(c1ccc(Cl)cc1)=N[C@@H](CC(=O)Nc1ccc3c(c1)C[C@@H]1CON=C31)c1nnc(C)n1-2]

The present disclosure also provides a pharmaceutical composition comprising a therapeutically effective amount of the above-mentioned compound, or an isomer or a pharmaceutically acceptable salt thereof, as an active ingredient and a pharmaceutically acceptable carrier.

In some embodiments disclosed herein, the use of the above-mentioned compound, or an isomer or a pharmaceutically acceptable salt thereof, or the above-mentioned composition in the manufacture of a medicament for treating diseases related to BET Bromodomain protein inhibition and PD-L1 gene expression is provided.

In some embodiments disclosed herein, the above-mentioned medicament is a medicament for treating tumors.

### Definitions and Terms

Unless otherwise specified, the following terms and phrases used herein are intended to have the following meanings. A specific term or phrase should not be considered indefinite or unclear in the absence of a particular definition, but should be understood in the conventional sense. When a trade name appears herein, it is intended to refer to its corresponding commodity or active ingredient thereof. The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, allergic reaction or other problems or complications, commensurate with a reasonable benefit/risk ratio.

The term “pharmaceutically acceptable salt” means a salt of the compound disclosed herein that is prepared by reacting the compound having a specific substituent disclosed herein with a relatively non-toxic acid or base. When the compound disclosed herein contains a relatively acidic functional group, a base addition salt can be obtained by bringing the neutral form of the compound into contact with a sufficient amount of base in a pure solution or a suitable inert solvent. The pharmaceutically acceptable base addition salt includes a salt of sodium, potassium, calcium, ammonium, organic amine or magnesium or similar salts. When the compound disclosed herein contains a relatively basic functional group, an acid addition salt can be obtained by bringing the neutral form of the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and an salt of amino acid (such as arginine and the like), and a salt of an organic acid such as glucuronic acid and the like. Certain specific compounds disclosed herein contain both basic and acidic functional groups and can be converted to any base or acid addition salt.

The pharmaceutically acceptable salt disclosed herein can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

In addition to the salt form, the compound provided herein also exists in prodrug form. The prodrug of the compound described herein is the compound that readily undergoes chemical change under physiological condition to be converted into the compound disclosed herein. Additionally, the prodrug can be converted to the compound disclosed herein by a chemical or biochemical method in vivo environment.

Certain compounds disclosed herein can exist in an unsolvated form or a solvated form, including a hydrated form. Generally, the solvated form is equivalent to the unsolvated form, and both are encompassed within the scope disclosed herein.

The compound disclosed herein may be present in a specific geometric or stereoisomeric form. The present disclosure contemplates all such compounds, including cis and trans isomer, (−)- and (+)-enantiomer, (R)- and (S)-enantiomer, diastereoisomer, (D)-isomer, (L)-isomer, and racemic mixture and other mixtures, for example, an enantiomer or diastereoisomer enriched mixture, all of which are encompassed within the scope disclosed herein. The substituent such as alkyl may have an additional asymmetric carbon atom. All these isomers and mixtures thereof are encompassed within the scope disclosed herein.

Unless otherwise specified, the term “enantiomer” or “optical isomer” means stereoisomers that are in a mirrored relationship with each other.

Unless otherwise specified, the term “cis-trans isomer” or “geometric isomer” is produced by the inability of a double bond or a single bond between ring-forming carbon atoms to rotate freely.

Unless otherwise specified, the term “diastereomer” means a stereoisomer in which two or more chiral centers of are contained in a molecule and is in a non-mirrored relationship between molecules.

Unless otherwise specified, “(D)” or “(+)” means dextroisomer, “(L)” or “(−)” means levoisomer, and “(DL)” or “(±)” means racemate.

Unless otherwise specified, a wedged solid bond ( [symbol] ) and a wedged dashed bond ( [symbol] ) indicate the absolute configuration of a stereocenter; a straight solid bond ( [symbol] ) and a straight dashed bond ( [symbol] ) indicate the relative configuration of a stereocenter; a wavy line ( [symbol] ) indicates a wedged solid bond ( [symbol] ) or a wedged dashed bond ( [symbol] ); or a wavy line ( [symbol] ) indicates a straight solid bond ( [symbol] ) and a straight dashed bond ( [symbol] ).

Unless otherwise specified, when a double bond structure such as a carbon-carbon double bond, a carbon-nitrogen double bond, and a nitrogen-nitrogen double bond is present in a compound, and each atom on the double bond is attached to two different substituents (in the double bond containing a nitrogen atom, a pair of lone pair electrons on the nitrogen atom is considered as one of the substituents to which it is attached), the compound represents (Z) isomer, (E) isomer, or a mixture of two isomers of the compound, if the atoms on the double bond in the compound are attached to their substituents by a wavy line ( [symbol] ). For example, the compound having following formula (A) means that the compound is present as a single isomer of formula (A-1) or formula (A-2) or as a mixture of two isomers of formula (A-1) and formula (A-2); and the compound having following formula (B) means that the compound is present as a single isomer of formula (B-1) or formula (B-2) or as a mixture of two isomers of formula (B-1) and formula (B-2). The compound having following formula (C) means that the compound is present as a single isomer of formula (C-1) or formula (C-2) or as a mixture of two isomers of formula (C-1) and formula (C-2).

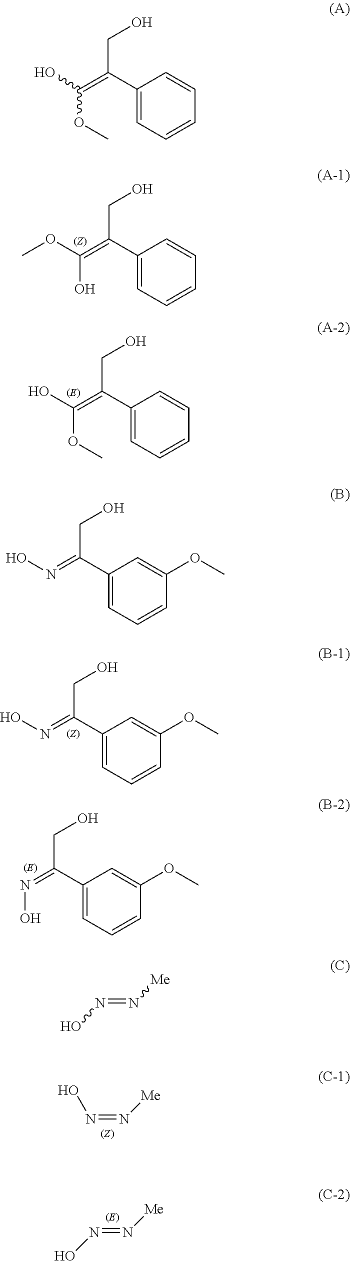

The compounds disclosed herein may be present in a particular form. Unless otherwise specified, the terms “tautomer” or “tautomeric form” means that different functional groups are in dynamic equilibrium at room temperature and can be rapidly converted into each other. If tautomers are possible (as in solution), a chemical equilibrium of tautomers can be achieved. For example, proton tautomers (also known as prototropic tautomers) include interconversions by proton transfer, such as keto-enol isomerization and imine-enamine isomerization. Valence tautomers include interconversions by recombination of some bonding electrons. A specific example of keto-enol tautomerization is interconversion between two tautomers pentane-2,4-dione and 4-hydroxypent-3-en-2-one.

Unless otherwise specified, the term “enriched in one isomer”, “isomer enriched”, “enriched in one enantiomer” or “enantiomeric enriched” means that the content of one isomer or enantiomer is less than 100%, and the content of the isomer or enantiomer is 60% or more, or 70% or more, or 80% or more, or 90% or more, or 95% or more, or 96% or more, or 97% or more, 98% or more, 99% or more, 99.5% or more, 99.6% or more, 99.7% or more, 99.8% or more, or 99.9% or more.

Unless otherwise specified, the term “isomer excess” or “enantiomeric excess” means the difference between the relative percentages of two isomers or two enantiomers. For example, if one isomer or enantiomer is present in an amount of 90% and the other isomer or enantiomer is present in an amount of 10%, the isomer or enantiomeric excess (ee value) is 80%.

Optically active (R)- and (S)-isomer, or D and L isomer can be prepared using chiral synthesis or chiral reagents or other conventional techniques. If one kind of enantiomer of certain compound disclosed herein is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to afford the pure enantiomer. In addition, the enantiomer and the diastereoisomer are generally isolated through chromatography which uses a chiral stationary phase and optionally combines with a chemical derivative method (for example, carbamate generated from amine).

The compounds disclosed herein may contain an unnatural proportion of atomic isotopes at one or more of the atoms that make up the compounds. For example, a compound may be labeled with a radioisotope such as tritium (³H), iodine-125 (¹²⁵I) or C-14 (¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs have advantages of reduced toxic side effects, increased drug stability, enhanced efficacy, and prolonged biological half-life of drugs. All changes in the isotopic composition of compounds disclosed herein, regardless of radioactivity, are included within the scope of the present disclosure.

The term “optional” or “optionally” means that the subsequent event or condition may occur but not requisite, that the term includes the instance in which the event or condition occurs and the instance in which the event or condition does not occur.

The term “substituted” means one or more than one hydrogen atom(s) on a specific atom are substituted by a substituent, including deuterium and hydrogen variants, as long as the valence of the specific atom is normal and the substituted compound is stable. When the substituent is oxo (i.e., ═O), it means two hydrogen atoms are substituted. Positions on an aromatic ring cannot be substituted by oxo.

The term “optionally substituted” means an atom can be substituted by a substituent or not, unless otherwise specified, the species and number of the substituent may be arbitrary as long as being chemically achievable.

When any variable (such as R) occurs in the constitution or structure of the compound more than once, the definition of the variable at each occurrence is independent. Thus, for example, if a group is substituted by 0-2 R, the group can be optionally substituted by up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variable is a single bond, it means that the two groups linked by the single bond are connected directly. For example, when L in A-L-Z represents a single bond, the structure of A-L-Z is actually A-Z.

When a substituent is vacant, it means that the substituent does not exist. For example, when X is vacant in A-X, the structure of A-X is actually A. When an enumerative substituent does not indicate through which atom it is linked to the substituted group, such substituent can be bonded through any of its atoms. For example, a pyridyl group as a substituent may be linked to the substituted group through any one of carbon atoms on the pyridine ring. When an enumerative linking group does not indicate its linking direction, its linking direction is arbitrary. For example, when the linking group L in

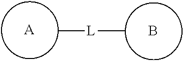

is -M-W—, the -M-W— can be linked to the ring A and the ring B in the same direction as the reading order from left to right to constitute

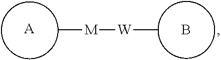

or can be linked to the ring A and the ring B in the reverse direction as the reading order from left to right to constitute

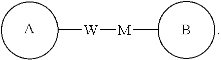

A combination of the linking groups, substituents and/or variants thereof is allowed only when such combination can result in a stable compound.

Unless otherwise specified, the term “C₁₋₆ alkyl” is used to indicate a linear or branched saturated hydrocarbon group consisting of 1 to 6 carbon atoms. The C₁₋₆ alkyl group includes C₁₋₅, C₁₋₄, C₁₋₃, C₁₋₂, C₂₋₆, C₂₋₄, C₆, and C₅ alkyl groups, and the like. It may be monovalent (e.g., methyl), divalent (e.g., methylene) or multivalent (e.g., methenyl). Examples of C₁₋₆ alkyl groups include, but are not limited to, methyl (Me), ethyl (Et), propyl (including n-propyl and isopropyl), butyl (including n-butyl, isobutyl, s-butyl and t-butyl), pentyl (including n-pentyl, isopentyl and neopentyl), hexyl, and the like.

Unless otherwise specified, the term “C₁₋₃ alkyl” is used to indicate a linear or branched saturated hydrocarbon group consisting of 1 to 3 carbon atoms. The C₁₋₃ alkyl group includes C₁₋₂ and C₂₋₃ alkyl groups and the like. It may be monovalent (e.g., methyl), divalent (e.g., methylene) or multivalent (e.g., methenyl). Examples of C₁₋₃ alkyl groups include, but are not limited to, methyl (Me), ethyl (Et), propyl (including n-propyl and isopropyl), and the like.

Unless otherwise specified, the term “C₁₋₆ alkoxy” means alkyl groups containing 1 to 6 carbon atoms and attached to the remainder of a molecule by an oxygen atom. The C₁_6 alkoxy group includes C₁₋₄, C₁₋₃, C₁₋₂, C₂₋₆, C₂₋₄, C₆, C₅, C₄, and C₃ alkoxy groups, and the like. Examples of C₁₋₆ alkoxy groups include, but are not limited to, methoxy, ethoxy, propoxy (including n-propoxy and isopropoxy), butoxy (including n-butoxy, isobutoxy, s-butoxy and t-butoxy), pentoxy (including n-pentoxy, isopropoxy and neopentoxy), hexyloxy, and the like.

Unless otherwise specified, the term “C₁₋₃ alkoxy” means alkyl groups containing 1 to 3 carbon atoms and attached to the remainder of a molecule by an oxygen atom. The C₁₋₃ alkoxy group includes C₁₋₂, C₂₋₃, C₃, and C₂ alkoxy groups, and the like. Examples of C₁₋₃ alkoxy groups include, but are not limited to, methoxy, ethoxy, propoxy (including n-propoxy and isopropoxy), and the like.

Unless otherwise specified, the term “C₁₋₆ alkylamino” means alkyl groups containing 1 to 6 carbon atoms and attached to the remainder of a molecule by an amino group. The C₁₋₆ alkylamino group includes C₁₋₄, C₁₋₃, C₁₋₂, C₂₋₆, C₂₋₄, C₆, C₅, C₄, C₃, and C₂ alkylamino groups, and the like. Examples of C₁₋₆ alkylamino groups include, but are not limited to —NHCH₃, —N(CH₃)₂, —NHCH₂CH₃, —N(CH₃)CH₂CH₃, —N(CH₂CH₃)(CH₂CH₃), —NHCH₂CH₂CH₃, —NHCH₂(CH₃)₂, —NHCH₂CH₂CH₂CH₃, and the like.

Unless otherwise specified, the term “C₁₋₃ alkylamino” means alkyl groups containing 1 to 3 carbon atoms and attached to the remainder of a molecule by an amino group. The C₁₋₃ alkylamino group includes C₁₋₂, C₃ and C₂ alkylamino groups and the like. Examples of C₁₋₃ alkylamino groups include, but are not limited to —NHCH₃, —N(CH₃)₂, —NHCH₂CH₃, —N(CH₃)CH₂CH₃, —NHCH₂CH₂CH₃, —NHCH₂(CH₃)₂, and the like.

Unless otherwise specified, the term “4- to 6-membered heterocycloalkyl” alone or in combination with other terms each means a saturated cyclic group consisting of 4 to 6 ring atoms, of which 1, 2, 3 or 4 ring atoms are heteroatoms independently selected from O, S and N, and the remainder atoms are carbon atoms, wherein the nitrogen atom is optionally quaternized and the nitrogen and sulfur heteroatoms are optionally oxidized (i.e., NO and S(O)ₚ, p is 1 or 2). The 4- to 6-membered heterocycloalkyl comprises a single ring system and a double ring system, wherein the double ring system comprises a sprio-ring, a fused-ring, and a bridge-ring. In addition, with respect to the “4- to 6-membered heterocycloalkyl”, the heteroatom may be present on the position of attachment of the heterocycloalkyl group to the remainder of a molecule. The 4- to 6-membered heterocycloalkyl group includes 5- to 6-membered, 4-membered, 5-membered, and 6-membered heterocycloalkyl groups, and the like. Examples of 4- to 6-membered heterocycloalkyl include, but are not limited to, azetidinyl, oxetanyl, thietanyl, pyrrolidinyl, pyrazolidinyl, imidazolidinyl, tetrahydrothienyl (including tetrahydrothien-2-yl and tetrahydrothien-3-yl and the like), tetrahydrofuranyl (including tetrahydrofuran-2-yl and the like), tetrahydropyranyl, piperidinyl (including 1-piperidinyl, 2-piperidinyl and 3-piperidinyl and the like), piperazinyl (including 1-piperazinyl and 2-piperazinyl and the like), morpholinyl (including 3-morpholinyl and 4-morpholinyl and the like), dioxanyl, dithianyl, isoxazolidinyl, isothiazolidinyl, 1,2-oxazinyl, 1,2-thiazinyl, hexahydropyridazinyl, homopiperazinyl, or homopiperidinyl and the like.

Unless otherwise specified, the term “5- to 6-membered heterocycloalkenyl” alone or in combination with other terms each means a partially unsaturated cyclic group containing at least one carbon-carbon double bond and consisting of 5 to 6 ring atoms, of which 1, 2, 3 or 4 ring atoms are heteroatoms independently selected from O, S and N, and the remainder atoms are carbon atoms, wherein the nitrogen atom is optionally quaternized and the nitrogen and sulfur heteroatoms are optionally oxidized (i.e., NO and S(O)ₚ, p is 1 or 2). The 4- to 6-membered heterocycloalkenyl comprises a single ring system and a double ring system, wherein the double ring system comprises a sprio-ring, a fused-ring and a bridge-ring, and any ring of the system is non-aromatic. In addition, with respect to the “5- to 6-membered heterocycloalkenyl”, the heteroatom may be present on the position of attachment of the heterocycloalkenyl group to the remainder of a molecule. The 5- to 6-membered heterocycloalkenyl group includes 5-membered and 6-membered heterocycloalkenyl groups and the like. Examples of 5- to 6-membered heterocycloalkenyl groups include, but are not limited to

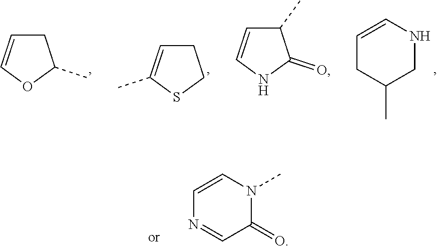

Unless otherwise specified, the terms “5- to 6-membered heteroaromatic ring” and “5- to 6-membered heteroaryl” may be used interchangeably. The term “5- to 6-membered heteroaryl” means a monocyclic group having a conjugated pi electron system and consisting of 5 to 6 ring atoms, of which 1, 2, 3 or 4 ring atoms are heteroatoms independently selected from O, S and N, and the remainder atoms are carbon atoms, wherein the nitrogen atom is optionally quaternized and the nitrogen and sulfur heteroatoms are optionally oxidized (i.e., NO and S(O)ₚ, p is 1 or 2). The 5- to 6-membered heteroaryl group may be attached to the remainder of a molecule by a heteroatom or a carbon atom. The 5- to 6-membered heteroaryl group includes 5-membered and 6-membered heteroaryl groups. Examples of the 5- to 6-membered heteroaryl group include, but are not limited to, pyrrolyl (including N-pyrrolyl, 2-pyrrolyl, 3-pyrrolyl, and the like), pyrazolyl (including 2-pyrazolyl and 3-pyrazolyl, and the like), imidazolyl (including N-imidazolyl, 2-imidazolyl, 4-imidazolyl, and 5-imidazolyl, and the like), oxazolyl (including 2-oxazolyl, 4-oxazolyl, and 5-oxazolyl, and the like), triazolyl (1H-1,2,3-triazolyl, 2H-1,2,3-triazolyl, 1H-1,2,4-triazolyl and 4H-1,2,4-triazolyl, and the like), tetrazolyl, isoxazolyl (3-isoxazolyl, 4-isoxazolyl and 5-isoxazolyl, and the like), thiazolyl (including 2-thiazolyl, 4-thiazolyl and 5-thiazolyl, and the like), furyl (including 2-furyl and 3-furyl, and the like), thienyl (including 2-thienyl and 3-thienyl, and the like), pyridyl (including 2-pyridyl, 3-pyridyl and 4-pyridyl, and the like), pyrazinyl or pyrimidinyl (including 2-pyrimidinyl and 4-pyrimidinyl, and the like).

Unless otherwise specified, Cₙ₋ₙ₊ₘ or Cₙ—Cₙ₊ₘ includes any one of n to n+m carbons. For example, C₁₋₁₂ includes Cl, C₂, C₃, C₄, C₅, C₆, C₇, C₈, C₉, C₁₀, C₁₁, and C₁₂. Cₙ₋ₙ₊ₘ or Cₙ—Cₙ₊ₘ also includes any range of n to n+m. For example, C₁₋₁₂ includes C₁₋₃, C₁₋₆, C₁₋₉, C₃₋₆, C₃₋₉, C₃₋₁₂, C₆₋₉, C₆₋₁₂, C₉₋₁₂, and the like. Similarly, the n-membered to n+m-membered ring means that the number of atoms on the ring is n to n+m. For example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring. The n-membered to n+m-membered ring also means that the number of atoms on the ring includes any range from n to n+m. For example, 3- to 12-membered ring includes 3- to 6-membered ring, 3- to 9-membered ring, 5- to 6-membered ring, 5- to 7-membered ring, 6- to 7-membered ring, 6- to 8-membered ring, and 6- to 10-membered ring, and the like.

The term “leaving group” refers to a functional group or atom which can be replaced by another functional group or atom through a substitution reaction (such as affinity substitution reaction). For example, representative leaving groups include triflate; chlorine, bromine and iodine; sulfonate group, such as mesylate, tosylate, p-bromobenzenesulfonate, p-toluenesulfonate and the like; acyloxy, such as acetoxy, trifluoroacetoxy and the like.

The term “protecting group” includes, but is not limited to “amino protecting group”, “hydroxy protecting group” or “thio protecting group”. The term “amino protecting group” refers to a protecting group suitable for blocking the side reaction on the nitrogen of an amino. Representative amino protecting groups include, but are not limited to: formyl; acyl, such as alkanoyl (e.g. acetyl, trichloroacetyl or trifluoroacetyl); alkoxycarbonyl, such as tert-butoxycarbonyl (Boc); arylmethoxycarbonyl such as benzyloxycarbonyl (Cbz) and 9-fluorenylmethoxycarbonyl (Fmoc); arylmethyl such as benzyl (Bn), trityl (Tr), 1,1-bis-(4′-methoxyphenyl)methyl; silyl such as trimethylsilyl (TMS) and tert-butyldimethylsilyl (TBS) and the like. The term “hydroxy protecting group” refers to a protecting group suitable for blocking the side reaction on hydroxy. Representative hydroxy protecting groups include, but are not limited to: alkyl such as methyl, ethyl and tert-butyl; acyl such as alkanoyl (e.g. acetyl); arylmethyl such as benzyl (Bn), p-methoxybenzyl (PMB), 9-fluorenylmethyl (Fm), and diphenylmethyl (benzhydryl, DPM); silyl such as trimethylsilyl (TMS) and tert-butyl dimethyl silyl (TBS) and the like.

The compound disclosed herein can be prepared by a variety of synthetic methods well known to the skilled in the art, including the following enumerative embodiment, the embodiment formed by the following enumerative embodiment in combination with other chemical synthesis methods and the equivalent replacement well known to the skilled in the art. The alternative embodiment includes, but is not limited to the embodiment disclosed herein.

The structures of the compounds of the present disclosure can be confirmed by conventional methods well known to those skilled in the art. If the present disclosure relates to an absolute configuration of a compound, the absolute configuration can be confirmed by conventional techniques in the art, such as single crystal X-Ray diffraction (SXRD). In the single crystal X-Ray diffraction (SXRD), the diffraction intensity data of the cultivated single crystal is collected using a Bruker D8 venture diffractometer with a light source of CuKα radiation in a scanning mode of φ/ω scan; after collecting the relevant data, the crystal structure is further analyzed by the direct method (Shelxs97) to confirm the absolute configuration.

All of the solvents used in the present disclosure are commercially available. The present disclosure employs the following abbreviations: aq represents water; HATU represents O-(7-azabenzotriazol-1-yl)-N,N,N′,N′-tetramethyluronium hexafluorophosphate; EDC represents N-(3-dimethylaminopropyl)-N′-ethylcarbodiimide hydrochloride; m-CPBA represents 3-chloroperoxybenzoic acid; eq represents equivalent or equivalence; CDI represents carbonyl diimidazole; DCM represents dichloromethane; PE represents petroleum ether; DIAD represents diisopropyl azodicarboxylate; DMF represents N,N-dimethylformamide; DMSO represents dimethyl sulfoxide; EtOAc represents ethyl acetate; EtOH represents ethanol; MeOH represents methanol; CBz represents benzyloxycarbonyl, which is an amino protecting group; BOC represents tert-butoxycarbonyl, which is an amino protecting group; HOAc represents acetic acid; NaCNBH₃ represents sodium cyanoborohydride; r.t. represents room temperature; O/N represents overnight; THF represents tetrahydrofuran; Boc₂O represents di-tert-butyldicarbonate; TFA represents trifluoroacetic acid; DIPEA represents diisopropylethylamine; SOCl₂ represents thionyl chloride; CS₂ represents carbon disulfide; TsOH represents p-toluenesulfonic acid; NFSI represents N-fluoro-N-(phenylsulfonyl)benzenesulfonamide; NCS represents 1-chloropyrrolidine-2,5-dione; n-Bu₄NF represents tetrabutylammonium fluoride; iPrOH represents 2-propanol; mp represents melting point; LDA represents lithium diisopropylamide; LiHMDS represents lithium hexamethyldisilazide; Xantphos represents 4,5-bis(diphenylphosphino)-9,9-dimethylxanthene; LiAlH₄ represents lithium aluminum hydride; Pd₂(dba)₃ represents tris(dibenzylideneacetone)dipalladium; Pd(dba)₂ represents bis(dibenzylideneacetone)palladium; Pd(dppf)Cl₂ represents [1,1′-bis(diphenylphosphino)ferrocene]dichloropalladium; Pd(PPh₃)₄ represents tetrakis(triphenylphosphine)palladium; IPA represents isopropyl alcohol; DEA represents diethylamine; DMAP represents 4-dimethylaminopyridine; T₃P represents 1-(n-propyl)phosphonic anhydride; TBAI represents tetrabutylammonium iodide; NBS represents N-bromosuccinimide; POCl₃ represents phosphorus oxychloride; NaBH₄ represents sodium borohydride; PPh₃ represents triphenylphosphine; HEPES represents 2-(4-(2-hydroxyethyl)piperazin-1-yl)ethanesulfonic acid; BSA represents bovine serum albumin; CHAPS represents 3-((3-cholamidopropyl)dimethylammonio)-1-propanesulfonic acid; and solutol represents polyethylene glycol-15 hydroxystearate.

Compounds are named according to general naming principles in the art or by ChemDraw® software, and commercially available compounds are named with their vendor directory names.

### Technical Effects

The present compounds can inhibit the activity of BET Bromodomain and regulate the expression of PD-L1 gene. The present compounds have good PK property, can remarkably down-regulate the expression of PD-L1 gene, and have significant tumor inhibition effect on an animal transplanted tumor model of MC38 mouse colon cancer cells.

### BRIEF DESCRIPTION OF THE DRAWINGS

FIG. 1: Effect of a compound of the present disclosure on PD-L1 expression level in MCF7 cells.

FIG. 2: Effect of a compound of the present disclosure on PD-L1 expression level in MDA-MB-231 cells.

FIG. 3: Effect of compound 4 on tumor volume of a mouse PAN02 pancreatic cancer tumor model.

FIG. 4: Effect of compound 4 on tumor volume of a mouse EMT-6 breast cancer tumor model.

### DETAILED DESCRIPTION OF THE INVENTION

The present disclosure is described in detail below by means of examples. However, it is not intended that these examples have any disadvantageous limitations to the present disclosure. The present disclosure has been described in detail herein, and the embodiments are also disclosed herein. It will be apparent to those skilled in the art that various changes and modifications may be made to the embodiments disclosed herein without departing from the spirit and scope disclosed herein.

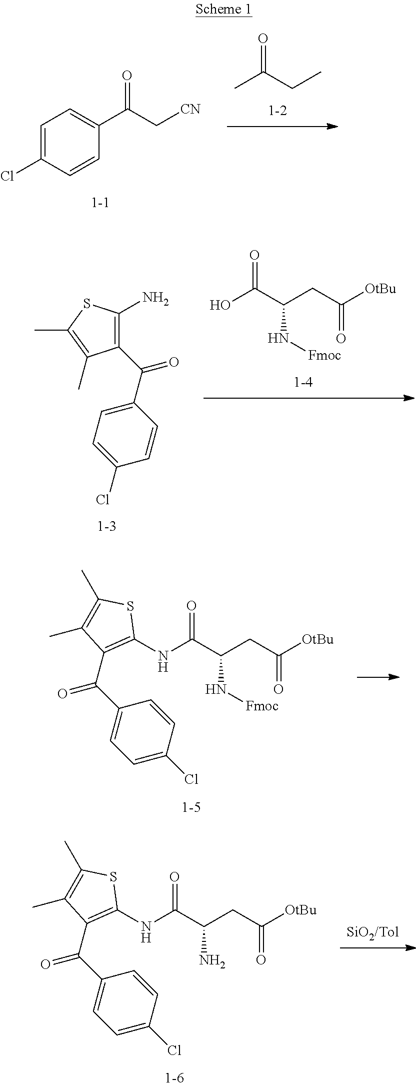

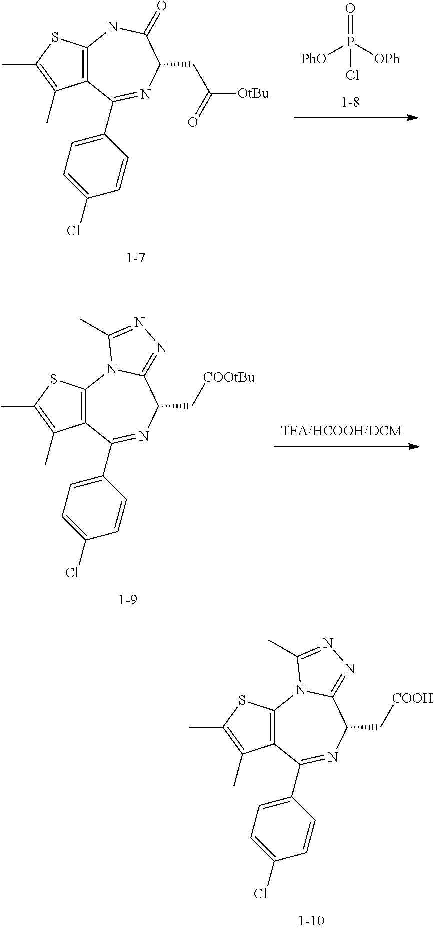

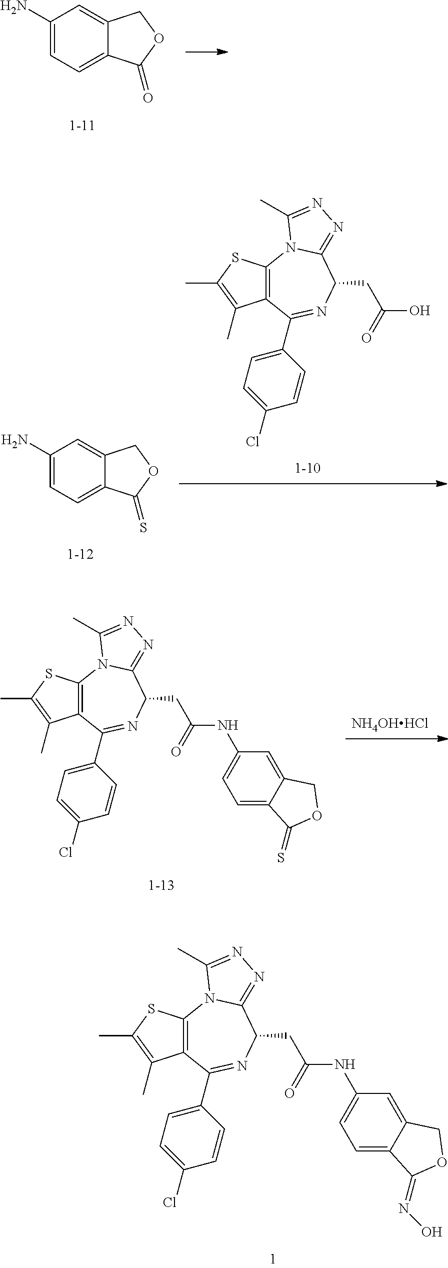 [SMILES: Nc1ccc2c(c1)COC2=O ; Nc1ccc2c(c1)COC2=S ; Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)Nc1ccc3c(c1)COC3=S)c1nnc(C)n1-2 ; "NH4OH·HCl" ; Cc1sc2c(c1C)C(c1ccc(Cl)cc1)=N[C@@H](CC(=O)Nc1ccc3c(c1)CO/C3=N\O)c1nnc(C)n1-2]

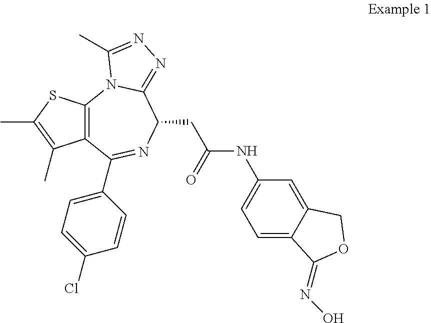 [SMILES: Cc1sc2c(c1C)C(c1ccc(Cl)cc1)=N[C@@H](CC(=O)Nc1ccc3c(c1)CO/C3=N\O)c1nnc(C)n1-2]

### Synthesis of Compound 1-3

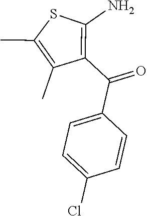 [SMILES: Cc1sc(N)c(C(=O)c2ccc(Cl)cc2)c1C]

Compound 1-1 (850 g, 4.73 mol, 1 eq) was added to a 5 L three-necked flask, to which i-PrOH (3400 mL) was then added at room temperature. To the reaction system were successively added compound 1-2 (344.66 g, 4.78 mol, 427.62 mL, 1.01 eq), and morpholine (461.79 g, 5.30 mol, 466.45 mL, 1.12 eq). The reaction was heated to 65° C. upon completion of the addition. Sublimed sulfur (160.88 g, 5.02 mol, 1.06 eq) was then added to the reaction system, and the reaction was stirred at 80° C. for 12 hours. TLC and LCMS showed that the starting materials were completely consumed and the target product was produced. The reaction solution was cooled to room temperature and was slowly added to a saturated saline (10 L) with stirring to precipitate a solid, which was then filtered. The obtained solid was dissolved in methyl tert-butyl ether (7 L), and insoluble substances were filtered off. The filtrate was concentrated under reduced pressure to dryness. A mixture of petroleum ether and ethyl acetate (petroleum ether/ethyl acetate=5/1, 2 L) was added to the concentrated dry solid, and the mixture was stirred at room temperature for 10 min. The mixture was filtered to afford a solid, which was then dissolved in n-heptane (2 L). The resulting mixture was stirred at room temperature for 10 min and then filtered to afford Compound 1-3.

¹HNMR (400 MHz, CDCl₃) δ=7.49-7.40 (m, 2H), 7.38-7.36 (m, 2H), 6.43 (brs, 2H), 2.14 (s, 3H), 1.56 (s, 3H). LCMS(ESI) m/z: 266.0 (M+1).

### Synthesis of Compound 1-5

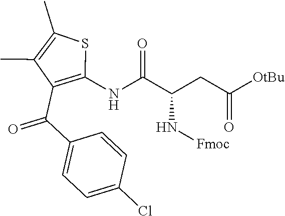

Under nitrogen protection, 1-3 (564 g, 2.12 mol, 1 eq) and pyridine (2200 mL) were added to a 5 L three-necked flask. The reaction was cooled to 0° C. and 1-4 (828.94 g, 2.02 mol, 0.95 eq) was added into the reaction. POCl₃ (390.21 g, 2.55 mol, 236.49 mL, 1.2 eq) was added into the reaction at 0-40° C. After the completion of the dropwise addition, the reaction was further stirred at room temperature (25° C.) for 1 hour. TLC and LCMS showed that the starting materials were completely consumed and the target product was produced. The reaction solution was slowly poured into a solution of 5N HCl (4.5 L) with stirring and a solid was precipitated. The resulting solid was filtered and the filter cake was washed with water (2 L). The filter cake was dispersed in 5 L of water. The mixture was stirred for 20 min, and then filtered. The filter cake was washed with water (2 L) to afford Compound 1-5. LCMS(ESI) m/z: 659.0 (M+1).

### Synthesis of Compound 1-6

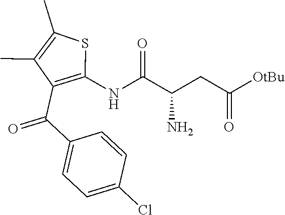 [SMILES: Cc1sc(NC(=O)[C@@H](N)CC(=O)OC(C)(C)C)c(C(=O)c2ccc(Cl)cc2)c1C]

Compound 1-5 (2.02 kg, 3.07 mol, 1 eq) and DMF (5.7 L) were added to a reaction flask, to which piperidine (621.94 g, 7.30 mol, 721.34 mL, 2.38 eq) was added slowly in batches. The reaction was further stirred at room temperature (25° C.) for 2 hours. LCMS showed that the starting materials were completely consumed and the target product was produced. The reaction solution was diluted with toluene (40 L). The mixture was washed with water (20 L*3) until the pH of the aqueous phase was ˜7, and then washed with saturated saline (20 L). The organic phase was dried over anhydrous sodium sulfate, and filtered. The filter cake was washed with anhydrous toluene (2 L). The filtrates were combined, and 4A molecular sieve was added to the combined filtrate. A 40 L toluene solution containing Compound 1-6 was obtained and used directly for the next reaction.

### Synthesis of Compound 1-7

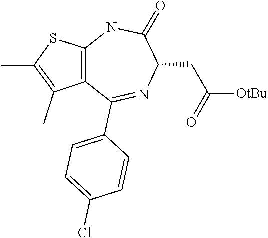 [SMILES: Cc1sc2c(c1C)C(c1ccc(Cl)cc1)=N[C@@H](CC(=O)OC(C)(C)C)C(=O)N2]

To a 50 L kettle were added the 40 L toluene solution containing Compound 1-6 from the above step and then SiO₂ (2.68 kg, 44.62 mol, 14.55 eq). The reaction was heated to 90° C. and stirred for 12 hours. LCMS showed that the starting materials were completely consumed. The reaction solution was cooled, and filtered. The filtrate was concentrated to afford concentrate 1 (about 900 g). The filter cake obtained in the above step was slurried with 12 L of ethyl acetate, filtered, and washed with ethyl acetate. The process was repeated 2 times. The filtrates were combined, and the combined filtrate was concentrated to afford concentrate 2 (about 400 g). The concentrate 1 was slurried with methyl tert-butyl ether (2.7 L, 1.8 L), and filtered. The filter cake was added to methyl tert-butyl ether (900 mL). The mixture was heated to 60° C., stirred for 1 hour, and then filtered while it was hot. The obtained filter cake was concentrated under reduced pressure to a constant weight to afford the filter cake 1. The concentrate 2 was slurried with methyl tert-butyl ether (800 mL), and filtered. The filter cake was added to methyl tert-butyl ether (400 mL). The mixture was heated to 60° C., stirred for 1 hour, and filtered while it was hot (the process was repeated three times). The filter cake was concentrated under reduced pressure to a constant weight to afford the filter cake 2. The resulting filter cake 1 and filter cake 2 was combined to afford Compound 1-7.

¹HNMR (400 MHz, DMSO-d6) δ=11.20 (s, 1H), 7.53-7.50 (m, 2H), 7.44-7.42 (m, 2H), 3.98-3.94 (m, 1H), 3.11-3.07 (m, 1H), 2.95-2.89 (m, 1H), 2.29 (s, 3H), 1.57 (s, 3H), 1.40 (s, 9H).

### Synthesis of Compound 1-9

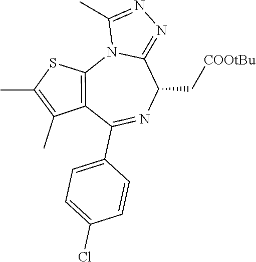

Under nitrogen protection, to a 5 L three-necked flask were successively added 1-7 and 2-methyltetrahydrofuran (3360 mL). The reaction was cooled to 0° C. T-BuOK (105.00 g, 935.70 mmol, 1.4 eq) was added to the reaction in batches at 0-5° C. After the addition, the reaction was further stirred at 0-5° C. for 2 hours. 1-8 (251.36 g, 935.70 mmol, 193.35 mL, 1.4 eq) was added to the reaction in batches. After the addition, the reaction was stirred at 0-5° C. for 1 hr. Acetyl hydrazine (99.03 g, 1.34 mol, 2 eq) was added to the reaction and the mixture was stirred at 0-5° C. for 1 hour. Then the reaction was heated to 70° C. and stirred for 12 hours. LCMS showed that the starting materials were not completely consumed and the target product was produced. The reaction solution was diluted with 4 L of ethyl acetate, and filtered. The filter cake was washed with 1 L of ethyl acetate. Water (4 L) was added to the filtrate, and solid sodium bicarbonate was added to adjust the pH of the solution to 8. The layers were separated. The aqueous phase was extracted with ethyl acetate (3 L), and the organic phases were combined, dried over anhydrous sodium sulfate, and filtered. The filtrate was concentrated, and the residue was purified by column chromatography to afford Compound 1-9.

¹HNMR (400 MHz, CDCl3) δ=7.41-7.39 (m, 2H), 7.33-7.31 (m, 2H), 4.57-4.54 (m, 1H), 3.59-3.53 (m, 2H), 2.69 (s, 3H), 2.14 (s, 3H), 1.65 (s, 3H), 1.53 (s, 9H). LCMS(ESI) m/z: 457.2 (M+1).

### Synthesis of Compound 1-10

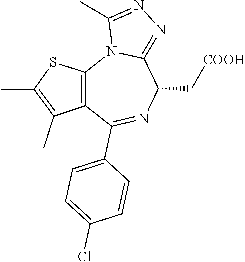 [SMILES: Cc1sc2c(c1C)C(c1ccc(Cl)cc1)=N[C@@H](CC(=O)O)c1nnc(C)n1-2]

To a reaction flask were added dichloromethane (8 mL), compound 1-9 (4.6 g, 10.07 mmol, 1 eq), formic acid (8 mL), and TFA (13 mL). The mixture was reacted at 15° C. for 36 hours. LCMS showed that the starting materials were completely consumed. The reaction solution was concentrated under reduced pressure to dryness. 20 mL of water and 30 mL of dichloromethane were added to the reaction, and a saturated aqueous solution of sodium bicarbonate was added to adjust the pH of the solution to 5. The layers were separated. The organic phase was washed once with 10 mL of saturated aqueous solution of sodium chloride. The organic phase was dried over anhydrous sodium sulfate, and filtered. The filtrate was concentrated to afford a crude product. The crude product was slurried successively with 8 mL of ethyl acetate, 8 mL of dichloromethane, and 8 mL of acetonitrile at room temperature for half an hour to afford Compound 1-10. LCMS(ESI) m/z: 401.1 (M+1).

### Synthesis of Compound 1-12

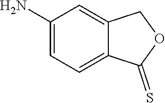 [SMILES: Nc1ccc2c(c1)COC2=S]

To a flask were successively added toluene (10 mL), Compound 1-11 (700 mg, 4.69 mmol, 1 eq) and 2,4-bis-(4-methoxyphenyl)-1,3-dithia-2,4-diphosphetan-2,4-disulfide (Lawson's reagent) (949.16 mg, 2.35 mmol, 0.5 eq). The reaction was heated to 110° C. for 18 hours, and then cooled to room temperature. 100 mL of water was added to the mixture, and the mixture was extracted with 20 mL of ethyl acetate. The combined organic phase was washed once with 10 mL of water and once with 10 mL of saturated aqueous solution of sodium chloride, respectively. The obtained organic phase was dried over anhydrous sodium sulfate, and filtered. The filtrate was concentrated to afford a crude product. The crude product was purified by column chromatography to afford compound 1-12. LCMS(ESI) m/z: 166.0 (M+1).

### Synthesis of Compound 1-13

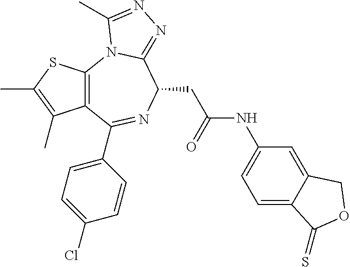 [SMILES: Cc1sc2c(c1C)C(c1ccc(Cl)cc1)=N[C@@H](CC(=O)Nc1ccc3c(c1)COC3=S)c1nnc(C)n1-2]

To a reaction flask were added dichloromethane (10 mL), Compound 1-12 (50 mg, 211.85 μmol, 1 eq), and Compound 1-10 (84.93 mg, 211.85 μmol, 1 eq). DMAP (2.59 mg, 21.18 μmol, 0.1 eq), T₃P (202.22 mg, 317.77 μmol, 188.99 μL, 50% purity, 1.5 eq) and DIPEA (51.94 mg, 401.88 μmol, 70.00 μL, 1.90 eq) were added successively to the mixture. The mixture was reacted at 20° C. for 18 hours. To the reaction system was added 10 mL of saturated aqueous solution of sodium bicarbonate. The mixture was extracted with 30 mL of dichloromethane. The layers were separated. The obtained organic phase was dried over anhydrous sodium sulfate, and filtered. The filtrate was concentrated under reduced pressure to afford a crude product. The crude product was purified by preparative TLC to afford compound 1-13. LCMS(ESI) m/z: 549.1 (M+1)

### Synthesis of Compound 1

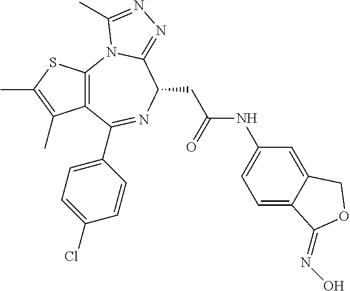 [SMILES: Cc1sc2c(c1C)C(c1ccc(Cl)cc1)=N[C@@H](CC(=O)Nc1ccc3c(c1)CO/C3=N\O)c1nnc(C)n1-2]

To a reaction flask were added EtOH (10 mL), Compound 1-13 (100 mg, 182.46 μmol, 1 eq), NH₂OH.HCl (38.04 mg, 547.37 μmol, 3 eq), and NaOAc (44.90 mg, 547.37 μmol, 3 eq). The mixture was stirred at 30° C. for 0.5 hours. The reaction solution was concentrated under reduced pressure to dryness and then purified by preparative HPLC (basic) to afford Compound 1.

¹HNMR (400 MHz, CDCl3) δ=9.59 (s, 1H), 7.89 (s, 1H), 7.53-7.51 (m, 1H), 7.46-7.40 (m, 2H), 7.39-7.32 (m, 3H), 6.62 (brs, 1H), 5.40 (s, 2H), 4.64-4.61 (m, 1H), 3.87-3.81 (m, 1H), 3.52-3.48 (m, 1H), 2.70 (s, 3H), 2.43 (s, 3H), 1.70 (s, 3H). LCMS(ESI) m/z: 547.1 (M+1).

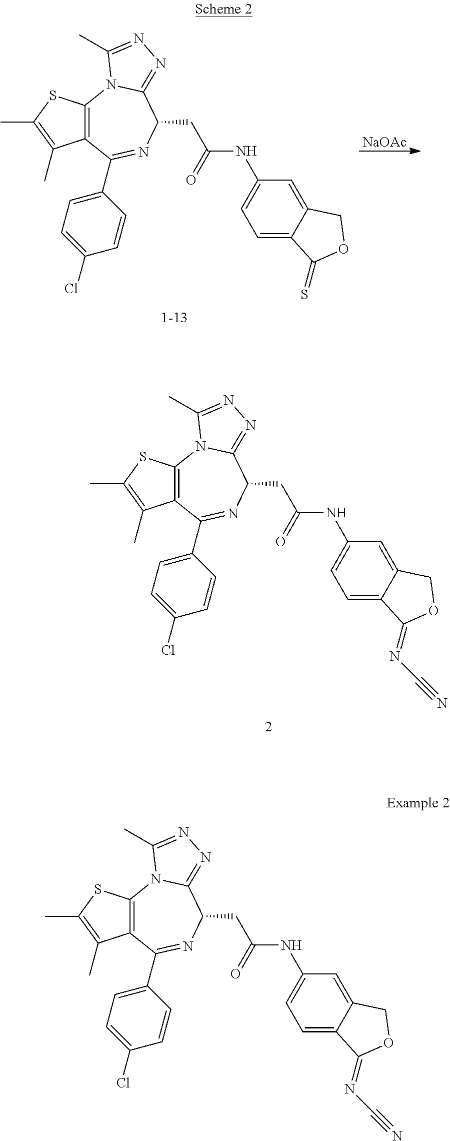 [SMILES: Cc1sc2c(c1C)C(c1ccc(Cl)cc1)=N[C@@H](CC(=O)Nc1ccc3c(c1)COC3=S)c1nnc(C)n1-2 ; Cc1sc2c(c1C)C(c1ccc(Cl)cc1)=N[C@@H](CC(=O)Nc1ccc3c(c1)CO/C3=N\C#N)c1nnc(C)n1-2 ; "NaOAc" ; Cc1sc2c(c1C)C(c1ccc(Cl)cc1)=N[C@@H](CC(=O)Nc1ccc3c(c1)CO/C3=N\C#N)c1nnc(C)n1-2]

Compound 1-13 (70 mg, 127.72 μmol, 1 eq), EtOH (42 mL), sodium acetate (52.39 mg, 638.60 μmol, 5 eq), and NH₂CN (42.95 mg, 510.88 μmol, 42.95 μL, 4 eq) were added to a reaction flask, and the mixture was stirred at 75° C. for 20 hours. The reaction solution was concentrated under reduced pressure, and the residue was purified by preparative HPLC (basic) to afford Compound 2.

¹HNMR (400 MHz, CDCl3) δ=10.18 (s, 1H), 8.13 (s, 1H), 7.757-7.75 (m, 1H), 7.44-7.42 (m, 2H), 7.34-7.27 (m, 3H), 5.52 (s, 2H), 4.62 (s, 1H), 3.84-3.82 (m, 1H), 3.55-3.53 (m, 1H), 2.71 (s, 3H), 2.44 (s, 3H), 1.72 (s, 3H). LCMS(ESI) m/z: 556.1 (M+1).

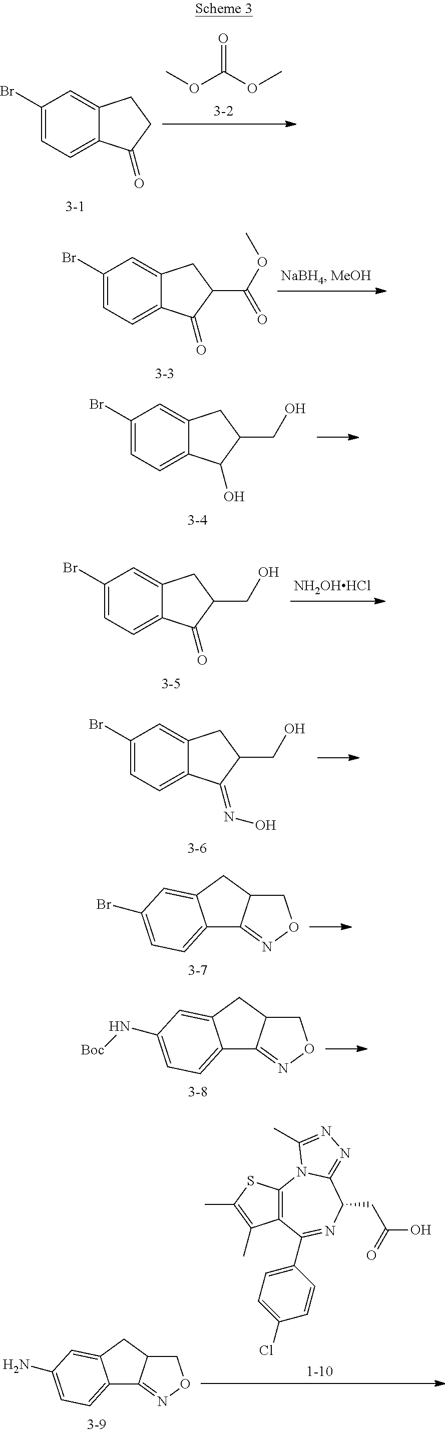

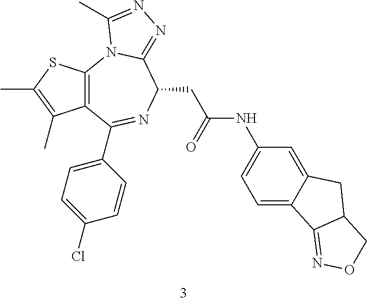 [SMILES: Cc1sc2c(c1C)C(c1ccc(Cl)cc1)=N[C@@H](CC(=O)Nc1ccc3c(c1)CC1CON=C31)c1nnc(C)n1-2]

### Example 3

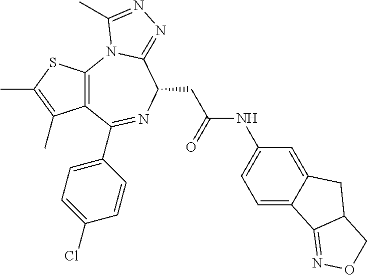 [SMILES: Cc1sc2c(c1C)C(c1ccc(Cl)cc1)=N[C@@H](CC(=O)Nc1ccc3c(c1)CC1CON=C31)c1nnc(C)n1-2]

### Synthesis of Compound 3-3

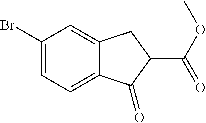

To a reaction flask was added Compound 3-1 (6 g, 28.43 mmol, 1 eq) in THF (120 mL), followed by NaH (3.41 g, 85.29 mmol, 60% purity, 3 eq) and Compound 3-2 (3.84 g, 42.64 mmol, 3.59 mL, 1.5 eq). The mixture was reacted at 65° C. for 2 hours. The system was cooled, and 50 mL saturated saline was added into the system to quench the reaction. Then 50 mL ethyl acetate was added into the system, and the layers were separated. The organic phase was dried over anhydrous sodium sulfate, and filtered. The filtrate was concentrated, and the residue was purified by column chromatography to afford compound 3-3. LCMS(ESI) m/z: 269.0, 270.9 (M+1, M+3).

### Synthesis of Compound 3-4

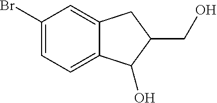 [SMILES: OCC1Cc2cc(Br)ccc2C1O]

To a reaction flask was added compound 3-3 (4 g, 14.86 mmol, 1 eq) in MeOH (40 mL), followed by NaBH₄ (4.50 g, 118.92 mmol, 8 eq). The mixture was reacted at 75° C. for 16 hours. The system was cooled, and then 40 mL of saturated ammonium chloride solution was added into the system. The mixture was concentrated. Then 50 mL of ethyl acetate was added into the system and the layers were separated. The organic phase was washed with 50 mL of saturated saline, dried over anhydrous sodium sulfate, and filtered. The filtrate was concentrated, and the residue was purified by column chromatography to afford compound 3-4. LCMS(ESI) m/z: 241.0, 243.0 (M-1).

### Synthesis of Compound 3-5

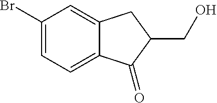 [SMILES: O=C1c2ccc(Br)cc2CC1CO]

To a reaction flask was added compound 3-4 (3 g, 12.34 mmol, 1 eq) in acetone (60 mL), and Jones reagent (2.5M, 9.87 mL, 2 eq) was added to the mixture at −78° C. The reaction was slowly warmed to 25° C. and stirred for 2 hours. 50 mL of isopropanol was added into the system, and the mixture was concentrated. 50 mL of water and 50 mL of ethyl acetate were added, and the layers were separated. The aqueous phase was extracted with ethyl acetate (50 mL*2), and the organic phases were combined. The organic phase was washed with 50 mL of saturated saline, dried over anhydrous sodium sulfate, and filtered. The filtrate was concentrated, and the residue was purified by column chromatography to afford Compound 3-5. LCMS(ESI) m/z: 241.0, 243.0 (M+1, M+3).

### Synthesis of Compound 3-6

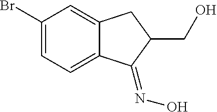 [SMILES: OCC1Cc2cc(Br)ccc2/C1=N/O]

To a reaction flask was added Compound 3-5 (1.2 g, 4.98 mmol, 1 eq) in MeOH (24 mL), followed by NH₂OH.HCl (518.85 mg, 7.47 mmol, 1.5 eq) and AcONa (612.49 mg, 7.47 mmol, 1.5 eq). The reaction mixture was stirred for 16 hours in an oil bath at 75° C. The system was concentrated, and then 10 mL of water and 10 mL of ethyl acetate were added into the system. The layers were separated. The organic phase was washed with saturated saline (10 mL*3), dried over anhydrous sodium sulfate, and filtered. The filtrate was concentrated, and the residue was purified by column chromatography to afford Compound 3-6. LCMS(ESI) m/z: 256.0, 258.0 (M+1, M+3).

### Synthesis of Compound 3-7

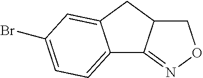 [SMILES: Brc1ccc2c(c1)CC1CON=C21]

To a reaction flask were added THF (60 mL) and Compound 3-6 (0.9 g, 3.51 mmol, 1 eq), followed by NaH (702.80 mg, 17.57 mmol, 60% purity, 5 eq) and p-toluenesulfonyl chloride (669.99 mg, 3.51 mmol, 1 eq). The reaction was stirred at 25° C. for 16 hours. Then 10 mL of saturated ammonium chloride solution was added into the system to quench the reaction. 10 mL of ethyl acetate was then added into the mixture. The layers were separated. The organic phase was washed with 10 mL of saturated saline, dried over anhydrous sodium sulfate, and filtered. The filtrate was concentrated, and the residue was purified by column chromatography to afford Compound 3-7. LCMS(ESI) m/z: 237.9, 239.9 (M+1, M+3).

### Synthesis of Compound 3-8

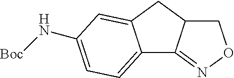 [SMILES: CC(C)(C)OC(=O)Nc1ccc2c(c1)CC1CON=C21]

To a reaction flask was added Compound 3-7 (0.04 g, 168.01 μmol, 1 eq) in toluene (2 mL), followed by tert-butyl carbamate (59.05 mg, 504.03 μmol, 3 eq), Cs₂CO₃ (136.85 mg, 420.03 μmol, 2.5 eq), Xantphos (19.44 mg, 33.60 μmol, 0.2 eq), and Pd₂(dba)₃ (15.39 mg, 16.80 μmol, 0.1 eq). The system was purged with nitrogen three times, and the mixture was stirred in an oil bath at 120° C. for 2 hours. To the system was added 10 mL of saturated saline. The mixture was extracted with ethyl acetate (10 mL*3), and the organic phase was dried over anhydrous sodium sulfate, and filtered. The filtrate was concentrated to afford Compound 3-8. LCMS(ESI) m/z: 275.1 (M+1).

### Synthesis of Compound 3-9

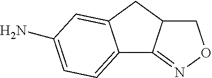 [SMILES: Nc1ccc2c(c1)CC1CON=C21]

To a reaction flask was added Compound 3-8 (0.09 g, 328.09 μmol, 1 eq), and the reaction was cooled to 0° C. TFA (1 mL) that was pre-cooled to 0° C. was then added into the system. After the completion of dropwise addition, the reaction mixture was slowly warmed to 25° C. and stirred for 1 hour. A proper amount of ice pieces was added into the system to quench the reaction. 10 mL of ethyl acetate was added into the mixture, and the mixture was adjusted to a pH of 7-8 with saturated sodium bicarbonate solution. The layers were separated after extraction. The organic phase was washed with 10 mL of saturated saline, dried over anhydrous sodium sulfate, and filtered. The filtrate was concentrated under reduced pressure to dryness. The residue was purified by preparative TLC to afford Compound 3-9. LCMS(ESI) m/z: 175.1 (M+1).

### Synthesis of Compound 3

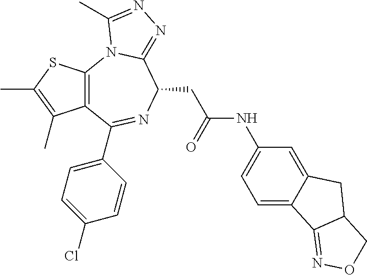 [SMILES: Cc1sc2c(c1C)C(c1ccc(Cl)cc1)=N[C@@H](CC(=O)Nc1ccc3c(c1)CC1CON=C31)c1nnc(C)n1-2]

To a reaction flask was added Compound 3-9 (14.08 mg, 80.82 μmol, 1.08 eq) in dichloromethane (1 mL), followed by Compound 1-10 (0.03 g, 74.84 μmol, 1 eq) and DMAP (914.25 μg, 7.48 μmol, 0.1 eq). The system was purged with nitrogen three times, and then DIPEA (13.35 mg, 103.27 μmol, 17.99 μL, 1.38 eq), and T₃P (33.81 mg, 106.27 μmol, 31.60 μL, 1.42 eq, 50% solution in ethyl acetate) were added to the system. The reaction was stirred at 20° C. for 1 hour. To the system were added 5 mL of a saturated sodium bicarbonate solution and 5 mL of dichloromethane. The layers were separated. The organic phase was washed with 10 mL of saturated saline, dried over anhydrous sodium sulfate, and filtered. The filtrate was concentrated, and the residue was purified by preparative HPLC (basic) to afford Compound 3.

¹HNMR (400 MHz, CDCl3) δ=9.82-9.37 (m, 1H), 7.84-7.82 (m, 1H), 7.63-7.54 (m, 1H), 7.46-7.39 (m, 2H), 7.39-7.29 (m, 3H), 4.84-4.54 (m, 2H), 4.00-3.72 (m, 3H), 3.61-3.41 (m, 1H), 3.25-3.02 (m, 1H), 2.71-2.70 (m, 4H), 2.43 (s, 3H), 1.71 (s, 3H). LCMS(ESI) m/z: 557.2 (M+1).

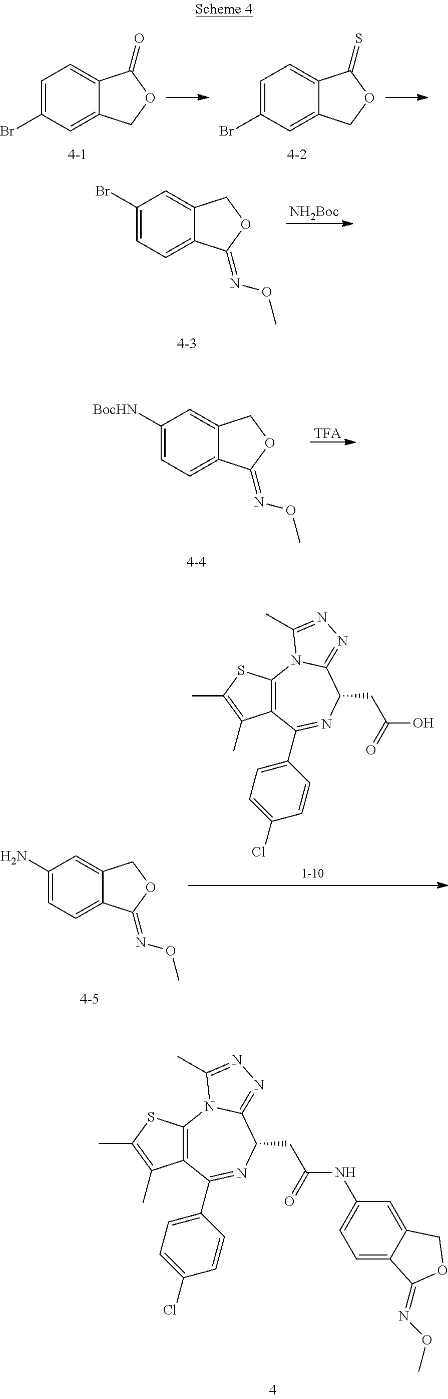

### Example 4

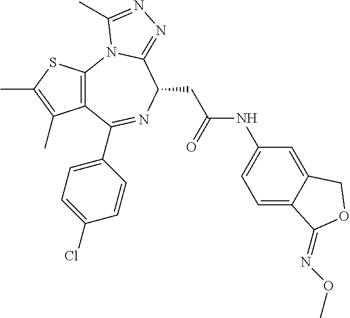 [SMILES: CO/N=C1\OCc2cc(NC(=O)C[C@@H]3N=C(c4ccc(Cl)cc4)c4c(sc(C)c4C)-n4c(C)nnc43)ccc21]

### Synthesis of Compound 4-2

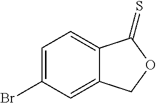 [SMILES: S=C1OCc2cc(Br)ccc21]

2,4-bis-(4-methoxyphenyl)-1,3-dithia-2,4-diphosphetan-2,4-disulfide (Lawson's reagent) (28.00 g, 69.23 mmol, 1.47 eq) was added to Compound 4-1 (10 g, 46.94 mmol, 1 eq) in toluene (200 mL). The reaction solution was heated to 120° C. and stirred for 12 hours. The reaction solution was cooled to room temperature and a large amount of solid was precipitated. The mixture was filtered, and the filter cake was washed with dichloromethane (20 mL*2). The filtrates were combined and concentrated to afford a crude product. The crude product was purified by column chromatography to afford Compound 4-2.

¹HNMR (400 MHz, CDCl3) δ=5.55 (s, 2H), 7.65-7.68 (m, 2H), 7.87-7.91 (m, 1H).

### Synthesis of Compound 4-3

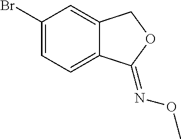

O-methylhydroxylamine hydrochloride (6.5 g, 77.83 mmol, 5.91 mL, 3.24 eq) and NaOAc (6.5 g, 79.24 mmol, 3.30 eq) were added to Compound 4-2 (5.5 g, 24.01 mmol, 1 eq) in EtOH (100 mL). The reaction solution was heated to 90° C. and stirred for 12 hours. The reaction solution was cooled to room temperature and concentrated. The resulting crude product was dissolved in dichloromethane (80 mL). The mixture was washed with water (50 mL*2). The organic phase was dried over anhydrous sodium sulfate, and filtered. The filtrate was concentrated to afford Compound 4-3. LCMS(ESI) m/z: 241.9, 243.9 (M+1, M+3).

### Synthesis of Compound 4-4

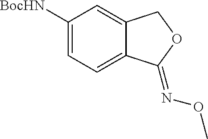 [SMILES: CO/N=C1\OCc2cc(NC(=O)OC(C)(C)C)ccc21]

To a solution of Compound 4-3 (5.1 g, 21.07 mmol, 1 eq) and tert-butyl carbamate (3.2 g, 27.32 mmol, 1.30 eq) in toluene (150 mL) were successively added Cs₂CO₃ (15 g, 46.04 mmol, 2.19 eq), Pd(dba)₂ (1 g, 1.74 mmol, 8.25 e-2 eq), 4,5-bis diphenyl phosphine 1 g, 1.73 mmol, 8.20 e-2 eq). The mixture was purged with nitrogen three times, heated to 120° C., and reacted for 1 hour. The reaction solution was cooled to room temperature, and filtered. The filter cake was washed with ethyl acetate (50 mL). The filtrate was concentrated to afford a crude product. The crude product was purified by column chromatography to afford Compound 4-4. LCMS(ESI) m/z: 278.8 (M+1).

### Synthesis of Compound 4-5

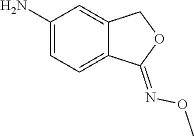

TFA (6.16 g, 54.02 mmol, 4 mL, 4.42 eq) was added to a solution of Compound 4-4 (3.4 g, 12.22 mmol, 1 eq) in dichloromethane (20 mL). The mixture was stirred at room temperature (26° C.) for 0.5 h. Dichloromethane (20 mL) and TFA (6.16 g, 54.02 mmol, 4 mL, 4.42 eq) were added into the mixture and the reaction was stirred at room temperature (26° C.) for 0.5 h. The reaction solution was adjusted to a pH of 7-8 with saturated aqueous NaHCO₃ solution. The reaction solution was allowed to stand, and the layers were separated. The organic phase was washed successively with saturated aqueous NaHCO₃ solution (40 mL) and water (40 mL), dried over anhydrous sodium sulfate, and filtered. The filtrate was concentrated to afford Compound 4-5.

¹HNMR (400 MHz, CDCl3) δ=3.82 (s, 3H), 3.92 (s, 2H), 5.25 (s, 2H), 6.49 (s, 1H), 6.59-6.61 (m, 1H), 7.39-7.41 (m, 1H). LCMS(ESI) m/z: 178.7 (M+1).

### Synthesis of Compound 4

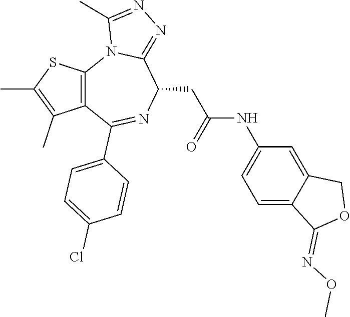 [SMILES: CO/N=C1\OCc2cc(NC(=O)C[C@@H]3N=C(c4ccc(Cl)cc4)c4c(sc(C)c4C)-n4c(C)nnc43)ccc21]

To Compound 1-10 (1.8 g, 4.49 mmol, 1 eq) in dichloromethane (50 mL) was added DIPEA (742.00 mg, 5.74 mmol, 1.00 mL, 1.28 eq). The system was purged with nitrogen three times. T₃P (3.75 g, 5.89 mmol, 3.50 mL, 50% purity, 1.31 eq), DMAP (500.00 mg, 4.09 mmol, 9.11 e-1 eq), and Compound 4-5 (1 g, 5.61 mmol, 1.25 eq) were added into the above reaction solution. The reaction solution was stirred at 30° C. for 1 hour. The reaction solution was then diluted with dichloromethane (20 mL) and washed successively with 1 M aqueous HCl solution (50 mL*2), saturated aqueous NaHCO₃ solution (50 mL*2) and water (50 mL*2). The organic phase was dried over anhydrous sodium sulfate, and filtered. The filtrate was concentrated to afford a crude product. The crude product was purified by column chromatography followed by preparative HPLC (basic) to afford Compound 4.

¹HNMR (400 MHz, CDCl3) δ=1.62-1.96 (s, 3H), 2.41 (s, 3H), 2.69 (s, 3H), 3.50 3.55 (m, 1H), 3.83-3.89 (m, 1H), 3.92 (s, 3H), 4.64-4.68 (m, 1H), 5.30-5.33 (m, 2H), 7.30-7.39 (m, 5H), 7.41-7.54 (m, 1H), 7.82 (s, 1H).9.79 (s, 1H). LCMS(ESI) m/z: 561.1 (M+1).

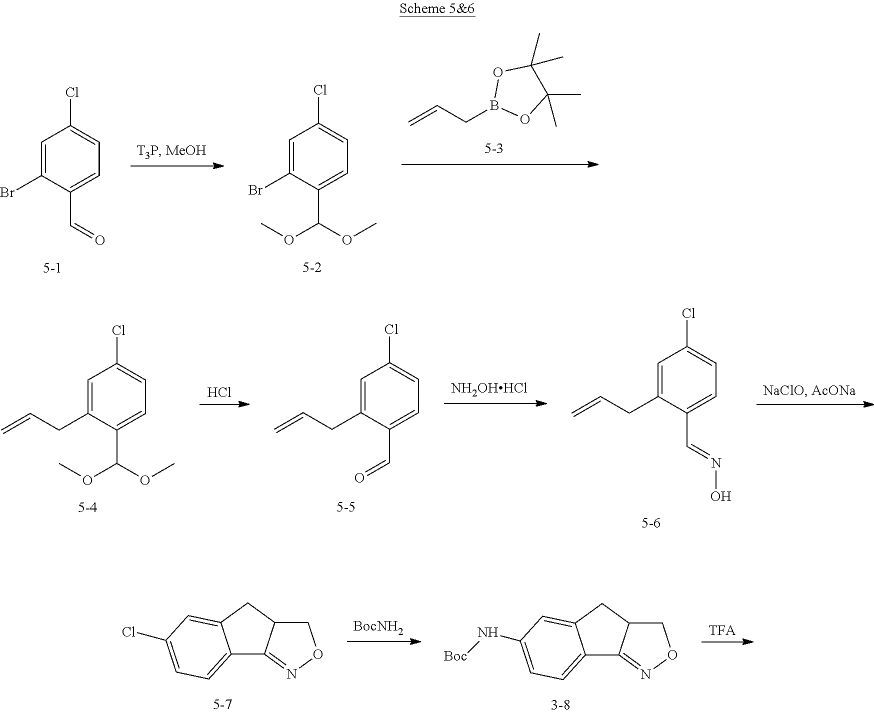

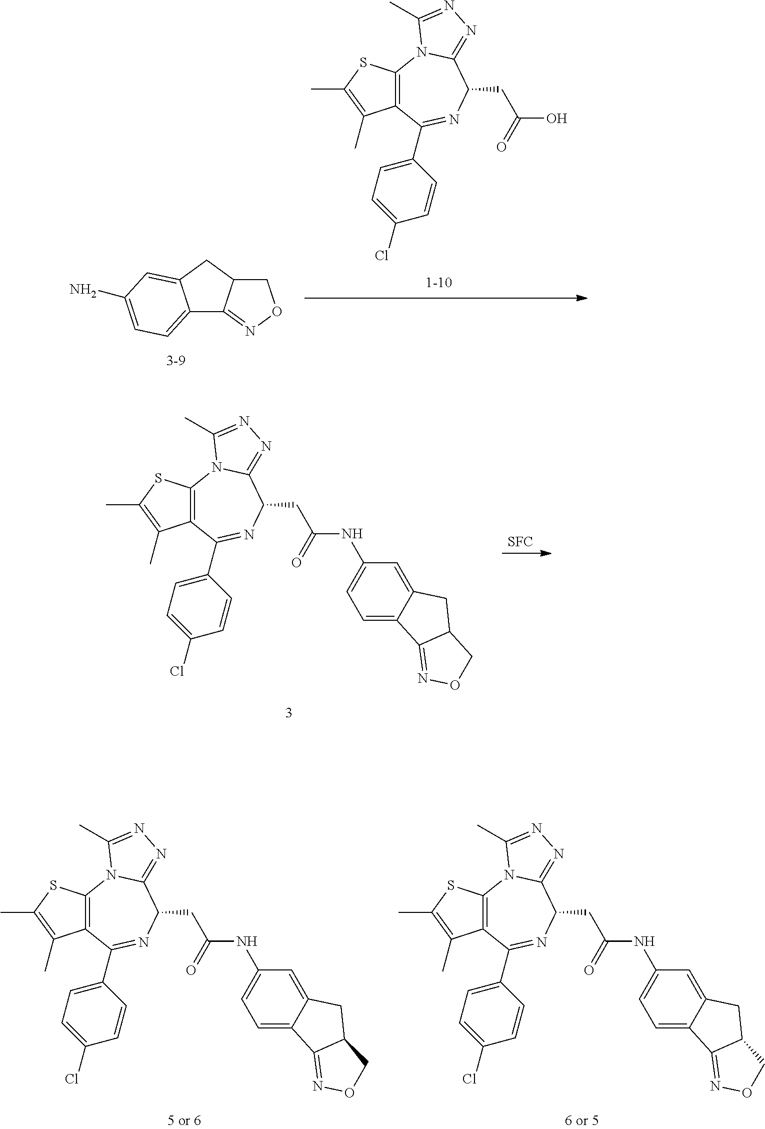 [SMILES: Cc1sc2c(c1C)C(c1ccc(Cl)cc1)=N[C@@H](CC(=O)O)c1nnc(C)n1-2 ; Nc1ccc2c(c1)CC1CON=C21 ; Cc1sc2c(c1C)C(c1ccc(Cl)cc1)=N[C@@H](CC(=O)Nc1ccc3c(c1)CC1CON=C31)c1nnc(C)n1-2 ; "SFC" ; Cc1sc2c(c1C)C(c1ccc(Cl)cc1)=N[C@@H](CC(=O)Nc1ccc3c(c1)C[C@H]1CON=C31)c1nnc(C)n1-2 ; Cc1sc2c(c1C)C(c1ccc(Cl)cc1)=N[C@@H](CC(=O)Nc1ccc3c(c1)C[C@@H]1CON=C31)c1nnc(C)n1-2]

### Examples 5&6

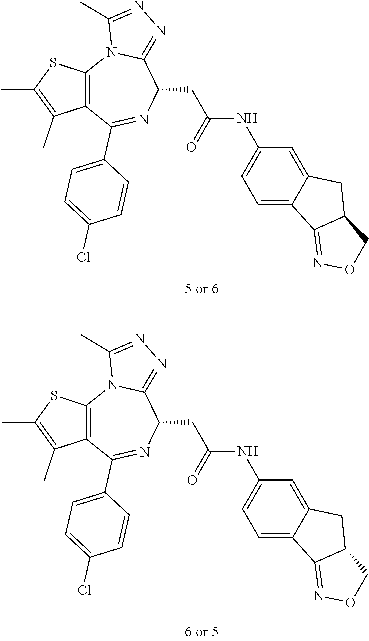 [SMILES: Cc1sc2c(c1C)C(c1ccc(Cl)cc1)=N[C@@H](CC(=O)Nc1ccc3c(c1)C[C@H]1CON=C31)c1nnc(C)n1-2 ; Cc1sc2c(c1C)C(c1ccc(Cl)cc1)=N[C@@H](CC(=O)Nc1ccc3c(c1)C[C@@H]1CON=C31)c1nnc(C)n1-2]

### Synthesis of Compound 5-2

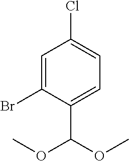

To a reaction flask was added Compound 5-1 (30 g, 136.70 mmol, 1 eq) in MeOH (180 mL), followed by T₃P (217.47 g, 341.74 mmol, 203.25 mL, 50% purity, 2.5 eq). The system was purged 3 times with nitrogen, and the reaction was stirred at room temperature (25° C.) for 16 hours. LCMS and TLC showed that the starting materials disappeared and a new material was produced. Saturated sodium bicarbonate solution was added into the system to adjust the pH to 7-8. The mixture was concentrated under reduced pressure. 200 mL of ethyl acetate was added into the system. After extraction, the layers were separated. The organic phase was washed with 200 mL of saturated saline, dried over anhydrous sodium sulfate, and filtered. The filtrate was concentrated to afford a crude product. The crude product was purified by column chromatography with neutral alumina of 200-300 meshes to afford Compound 5-2. LCMS(ESI) m/z: 232.9, 234.9 (M-31).

### Synthesis of Compound 5-4

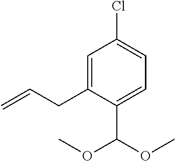

To a reaction flask were added Compound 5-2 (33 g, 124.28 mmol, 1 eq) and Compound 5-3 (52.21 g, 310.70 mmol, 2.5 eq) in THF (350 mL), followed by H₂O (90 mL), Na₂CO₃ (26.34 g, 248.56 mmol, 2 eq), and Pd(dppf)Cl₂ (18.19 g, 24.86 mmol, 0.2 eq). The system was purged three times with nitrogen, and the reaction was stirred in an oil bath at 80° C. for 16 hours. LCMS showed that the starting materials were completely consumed and the target product was produced. The reaction solution was directly used to the next step without further work up. LCMS(ESI) m/z: 227.1 (M+1).

### Synthesis of Compound 5-5

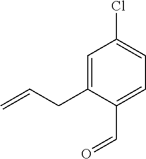

To the reaction solution of Compound 5-4 (33 g, 145.57 mmol, 1 eq) from the above step was added dilute HCl solution (1M, 330 mL, 2.27 eq), and the mixture was stirred at room temperature (25° C.) for 3 hours. LCMS and TLC showed that there were starting materials remaining and a new material was produced. 300 mL of ethyl acetate was added into the system. The mixture was emulsified and filtered. The layers were separated, and the organic phase was washed with 300 mL of saturated saline, dried over anhydrous sodium sulfate, and filtered. The filtrate was concentrated to afford a crude product. The crude product was purified by column chromatography to afford Compound 5-5.

¹HNMR (400 MHz, CDCl3) δ=10.23-10.16 (m, 1H), 7.79-7.77 (m, 1H), 7.57-7.53 (m, 1H), 7.38-7.29 (m, 1H), 6.03-5.96 (m, 1H), 5.15-5.12 (m, 1H), 5.06-5.03 (dm, 1H), 3.82-3.78 (m, 2H). LCMS(ESI) m/z: 181.0 (M+1).

### Synthesis of Compound 5-6

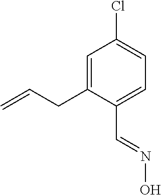

To a reaction flask were added H₂O (21 mL), NaHCO₃ (2.44 g, 29.06 mmol, 1.13 mL, 1.25 eq), and NH₂OH.HCl (2.02 g, 29.06 mmol, 1.25 eq). Compound 5-5 (4.2 g, 23.25 mmol, 1 eq) in MeOH (21 mL) was added into the mixture at 0° C. The reaction was stirred at room temperature (25° C.) for 3 hours. TLC and LCMS showed that the starting materials were completely consumed and the target product was produced. 50 mL of water was added into the system. The reaction solution was concentrated. Then 100 mL of ethyl acetate was added into the concentrate. The layers were separated. The organic phase was washed with 100 mL of saturate saline, dried over anhydrous sodium sulfate, and filtered. The filtrate was concentrated to afford a crude product. The crude product was purified by column chromatography to afford Compound 5-6. LCMS(ESI) m/z: 196.0 (M+1).

### Synthesis of Compound 5-7

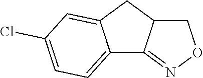 [SMILES: Clc1ccc2c(c1)CC1CON=C21]

To a reaction flask were successively added 5-6 (3.75 g, 19.17 mmol, 1 eq) in dichloromethane (80 mL), NaClO (40.77 g, 38.33 mmol, 33.69 mL, 7% purity, 2 eq) and AcONa (786.19 mg, 9.58 mmol, 0.5 eq). The mixture was stirred at 25° C. for 16 hours. LCMS and TLC showed that the starting materials were completely consumed and the target product was produced. Then 50 mL of dichloromethane and 50 mL of water were added into the system. After extraction, the layers were separated. The organic phase was washed with 100 mL of saturated ammonium chloride solution, dried over anhydrous sodium sulfate, and filtered. The filtrate was concentrated to afford a crude product. The crude product was purified by column chromatography to afford compound 5-7.

¹HNMR (400 MHz, CDCl3) δ=7.68-7.66 (m, 1H), 7.37-7.33 (m, 2H), 4.74-4.70 (m, 1H), 4.00-3.94 (m, 1H), 3.88-3.82 (m, 1H), 3.23-3.17 (m, 1H), 2.81-2.75 (m, 1H). LCMS(ESI) m/z: 194.0 (M+1).

### Synthesis of Compound 3-8

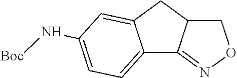 [SMILES: CC(C)(C)OC(=O)Nc1ccc2c(c1)CC1CON=C21]

To a reaction flask was added compound 5-7 (0.5 g, 2.58 mmol, 1 eq) in 1,4-dioxane (5 mL), followed by Cs₂CO₃ (1.18 g, 3.62 mmol, 1.4 eq), tert-butyl carbamate (363.00 mg, 3.10 mmol, 1.2 eq), 2-dicyclohexylphosphino-2′,4′,6′-triisopropylbiphenyl (443.16 mg, 929.61 μmol, 0.36 eq), and Pd(dba)₂ (178.18 mg, 309.87 μmol, 0.12 eq). The system was purged three times with nitrogen. The mixture was stirred in an oil bath at 105° C. for 2.5 hours. LCMS and TLC showed that the starting materials were completely consumed and the target product was produced. The system was cooled, and 20 mL of water and 20 mL of ethyl acetate were added into the system. The layers were separated. The organic phase was washed with 20 mL of saturated saline, dried over anhydrous sodium sulfate, and filtered. The filtrate was concentrated, and the residue was purified by column chromatography to afford Compound 3-8. LCMS(ESI) m/z: 275.1 (M+1).

### Synthesis of Compound 3-9

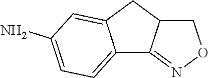 [SMILES: Nc1ccc2c(c1)CC1CON=C21]

To a reaction flask was added Compound 3-8 (0.37 g, 1.35 mmol, 1 eq) in dichloromethane (1 mL), and the mixture was cooled to 0° C. TFA (768.98 mg, 6.74 mmol, 499.34 μL, 5 eq) was then added, and the mixture was stirred at room temperature (25° C.) for 5 hours. TLC and LCMS showed that the starting materials were completely consumed and a new material was produced. A proper amount of ice pieces was added into the system to quench the reaction. Then 10 mL of ethyl acetate was added to the mixture, and the pH of the mixture was adjusted to be 7-8 by saturated sodium bicarbonate. The layers were separated. The organic phase was washed with 10 mL of saturated saline, dried over anhydrous sodium sulfate, and filtered. The filtrate was concentrated, and the residue was purified by column chromatography to afford Compound 3-9.

¹HNMR (400 MHz, CDCl3) δ=7.52-7.45 (m, 1H), 6.81-6.57 (m, 2H), 4.60-4.56 (m, 1H), 3.91-3.83 (m, 1H), 3.73-3.67 (m, 1H), 3.07-3.02 (m, 1H), 2.73 (s, 2H), 2.64 (dd, J=7.4, 15.6 Hz, 1H). LCMS(ESI) m/z: 175.1 (M+1).

### Synthesis of Compound 3

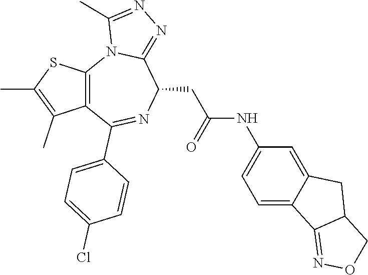 [SMILES: Cc1sc2c(c1C)C(c1ccc(Cl)cc1)=N[C@@H](CC(=O)Nc1ccc3c(c1)CC1CON=C31)c1nnc(C)n1-2]

To a reaction flask was added Compound 3-9 (0.1 g, 574.06 μmol, 1.1 eq) in dichloromethane (2 mL), followed by Compound 1-10 (209.21 mg, 521.87 μmol, 1 eq), and DMAP (6.38 mg, 52.19 μmol, 0.1 eq). The system was purged 3 times with nitrogen. To the mixture were successively added DIPEA (93.08 mg, 720.18 μmol, 125.44 μL, 1.38 eq), and T₃P (235.79 mg, 741.06 μmol, 220.36 μL, 1.42 eq, 50% solution in ethyl acetate). The reaction was stirred at room temperature (25° C.) for 3 hours. LCMS showed that the starting materials were completely consumed and the target product was produced. To the system were added 10 mL of a saturated sodium bicarbonate solution, and 10 mL of dichloromethane. The layers were separated. The organic phase was washed with 10 mL of saturated saline, dried over anhydrous sodium sulfate, and filtered. The filtrate was concentrated to afford a crude product. The crude product was purified by preparative HPLC (basic) to afford Compound 3. LCMS(ESI) m/z: 557.2 (M+1).

### Synthesis of Compounds 5&6

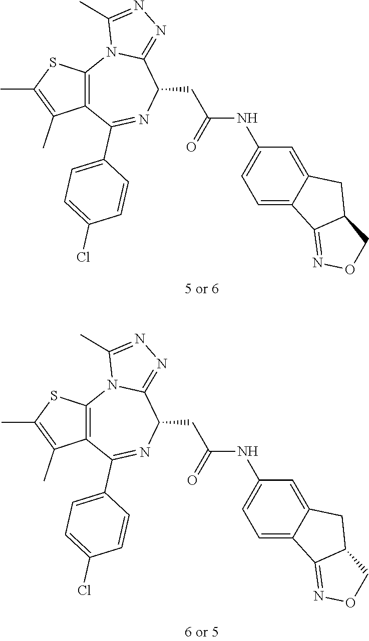 [SMILES: Cc1sc2c(c1C)C(c1ccc(Cl)cc1)=N[C@@H](CC(=O)Nc1ccc3c(c1)C[C@H]1CON=C31)c1nnc(C)n1-2 ; Cc1sc2c(c1C)C(c1ccc(Cl)cc1)=N[C@@H](CC(=O)Nc1ccc3c(c1)C[C@@H]1CON=C31)c1nnc(C)n1-2]

Compound 3 (0.18 g, 323.12 μmol, 1 eq) was subjected to SFC (chromatography column: DAICEL CHIRALPAK AS (250 mm*50 mm, 10 μm); mobile phase: [neutral-MeOH]; B (methanol) %: 40%-40%, 12 min) to be separated into two single configuration compounds. Compound 5 (Rt=1.893 min) and compound 6 (Rt=2.22 min) were afforded.

Compound 5 (Rt=1.893 min)

¹HNMR (400 MHz, CDCl3) δ=9.60 (s, 1H), 7.82 (s, 1H), 7.57-7.55 (m, 1H), 7.43-7.41 (m, 2H), 7.34-7.32 (m, 2H), 7.28 (brs, 1H), 4.70-4.65 (m, 2H), 3.9-3.82 (m, 3H), 3.52-3.47 (m, 1H), 3.10-3.09 (m, 1H), 2.71-2.66 (m, 4H), 2.43 (s, 3H), 1.71 (s, 3H).

LCMS(ESI) m/z: 557.2 (M+1).

Compound 6 (Rt=2.22 min)

¹HNMR (400 MHz, CDCl3) δ=9.55 (s, 1H), 7.79 (s, 1H), 7.62-7.60 (m, 1H), 7.43-7.40 (m, 2H), 7.38-7.32 (m, 2H), 7.29 (brs, 1H), 4.68-4.62 (m, 2H), 3.90-3.79 (m, 3H), 3.53-3.50 (m, 1H), 3.13-3.11 (m, 1H), 2.73-2.70 (m, 4H), 2.42 (s, 3H), 1.70 (s, 3H).

LCMS(ESI) m/z: 557.2 (M+1).

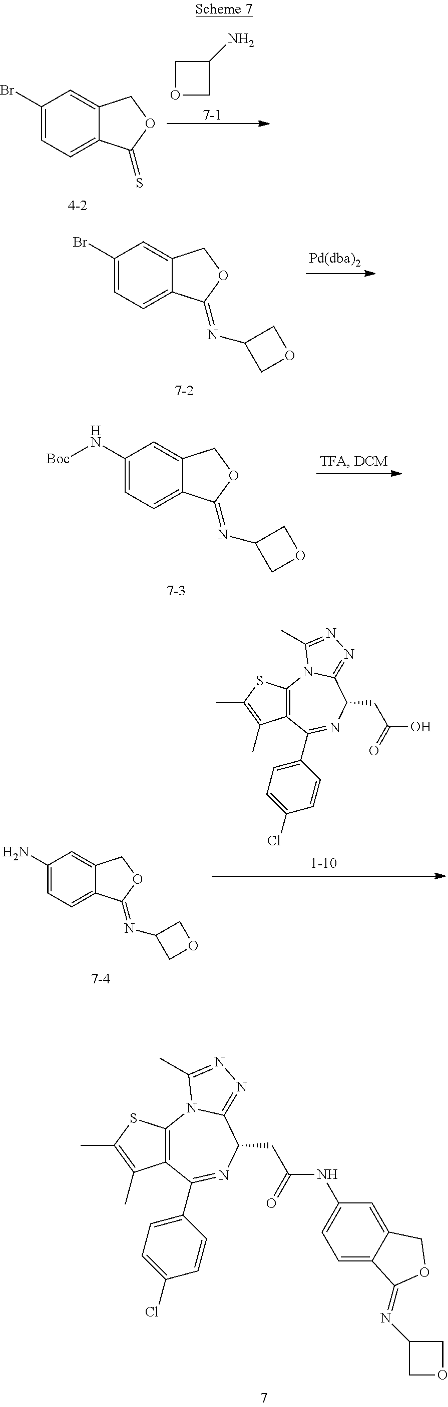

### Example 7

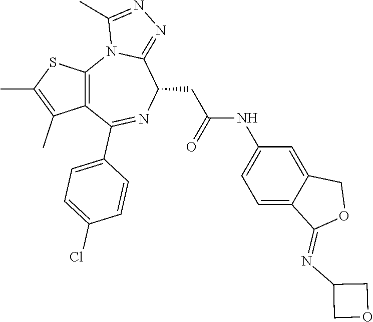 [SMILES: Cc1sc2c(c1C)C(c1ccc(Cl)cc1)=N[C@@H](CC(=O)Nc1ccc3c(c1)CO/C3=N\C1COC1)c1nnc(C)n1-2]

### Synthesis of Compound 7-2

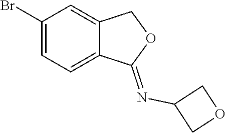 [SMILES: Brc1ccc2c(c1)CO/C2=N\C1COC1]

To a reaction flask were added Compound 4-2 (0.3 g, 1.31 mmol, 1 eq) in EtOH (3 mL) and Compound 7-1 (215.19 mg, 1.96 mmol, 1.5 eq), followed by AcONa (322.27 mg, 3.93 mmol, 3 eq). The reaction was stirred at room temperature (25° C.) for 3 hours. TLC showed that the starting points disappeared and a new point appeared. The system was concentrated to afford a crude product. The crude product was purified by column chromatography to afford Compound 7-2. LCMS(ESI) m/z: 267.9, 270.0 (M+1, M+3).

### Synthesis of Compound 7-3

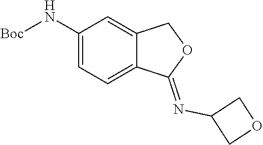 [SMILES: CC(C)(C)OC(=O)Nc1ccc2c(c1)CO/C2=N\C1COC1]

To a reaction flask was added Compound 7-2 (0.18 g, 671.38 μmol, 1 eq) in 1,4-dioxane (2 mL). To the reaction flask were successively added tert-butyl carbamate (235.95 mg, 2.01 mmol, 3 eq), Cs₂CO₃ (546.87 mg, 1.68 mmol, 2.5 eq), 2-dicyclohexylphosphino-2′,4′,6′-triisopropylbiphenyl (64.01 mg, 134.28 μmol, 0.2 eq), and Pd(dba)2 (38.60 mg, 67.14 μmol, 0.1 eq). The system was purged three times with nitrogen. The reaction was heated to 105° C. and stirred for 3 hours. LCMS and TLC showed that the starting materials were completely consumed and the target product was produced. To the system were added 10 mL of water and 10 mL of ethyl acetate. After extraction, the layers were separated. The organic phase was washed with 10 mL of saturated saline, dried over anhydrous sodium sulfate, and filtered. The filtrate was concentrated, and the residue was purified by column chromatography to afford Compound 7-3. LCMS(ESI) m/z: 305.1 (M+1).

### Synthesis of Compound 7-4

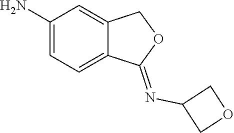 [SMILES: Nc1ccc2c(c1)CO/C2=N\C1COC1]

To a reaction flask was added Compound 7-3 (0.1 g, 328.58 μmol, 1 eq) in dichloromethane (1 mL), and the mixture was cooled to 0° C. TFA (1.54 g, 13.51 mmol, 1 mL, 41.10 eq) that was pre-cooled to 0° C. was added. The reaction was warmed to room temperature (25° C.) and stirred for 2 hours. TLC showed that the starting points disappeared and a new point appeared. A proper amount of ice pieces was added into the system to quench the reaction, and then saturated sodium bicarbonate solution was added to adjust the pH to be 7-8. 10 mL of dichloromethane was added to the mixture. After extraction, the layers were separated. The obtained organic phase was washed with 10 mL of saturated saline, dried over anhydrous sodium sulfate, and filtered. The filtrate was concentrated under reduced pressure. The residue was purified by column chromatography to afford Compound 7-4. LCMS(ESI) m/z: 205.1 (M+1).

### Synthesis of Compound 7

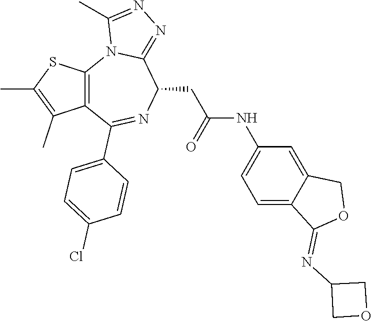 [SMILES: Cc1sc2c(c1C)C(c1ccc(Cl)cc1)=N[C@@H](CC(=O)Nc1ccc3c(c1)CO/C3=N\C1COC1)c1nnc(C)n1-2]

To a reaction flask was added Compound 7-4 (25.00 mg, 122.41 μmol, 1.1 eq) in dichloromethane (1 mL), followed by Compound 1-10 (44.61 mg, 111.29 μmol, 1 eq), and DMAP (1.36 mg, 11.13 μmol, 0.1 eq). The system was purged three times with nitrogen. To the reaction were successively added DIPEA (19.85 mg, 153.57 μmol, 26.75 μL, 1.38 eq), and T₃P (100.56 mg, 158.03 μmol, 93.98 μL, 50% purity, 1.42 eq). The reaction was stirred at room temperature (25° C.) for 3 hours. LCMS showed that the starting materials were completely consumed and the target product was produced. To the system were added 10 mL of saturated sodium bicarbonate solution and 10 mL of dichloromethane. After extraction, the layers were separated. The obtained organic phase was washed with 10 mL of saturated salt, dried over anhydrous sodium sulfate, and filtered. The filtrate was concentrated under reduced pressure to afford a crude product. The crude product was purified by preparative HPLC (basic) to afford Compound 7.

¹HNMR (400 MHz, CDCl3) δ=9.60 (s, 1H), 7.88 (s, 1H), 7.73-7.71 (m, 1H), 7.44-7.42 (m, 2H), 7.37-7.33 (m, 3H), 5.20 (s, 2H), 5.01-4.93 (m, 3H), 4.76-4.73 (m, 2H), 4.64-4.62 (m, 1H), 3.87-3.81 (m, 1H), 3.53-3.48 (m, 1H), 2.70 (s, 3H), 2.43 (s, 3H), 1.71 (s, 3H). LCMS(ESI) m/z: 587.2 (M+1).

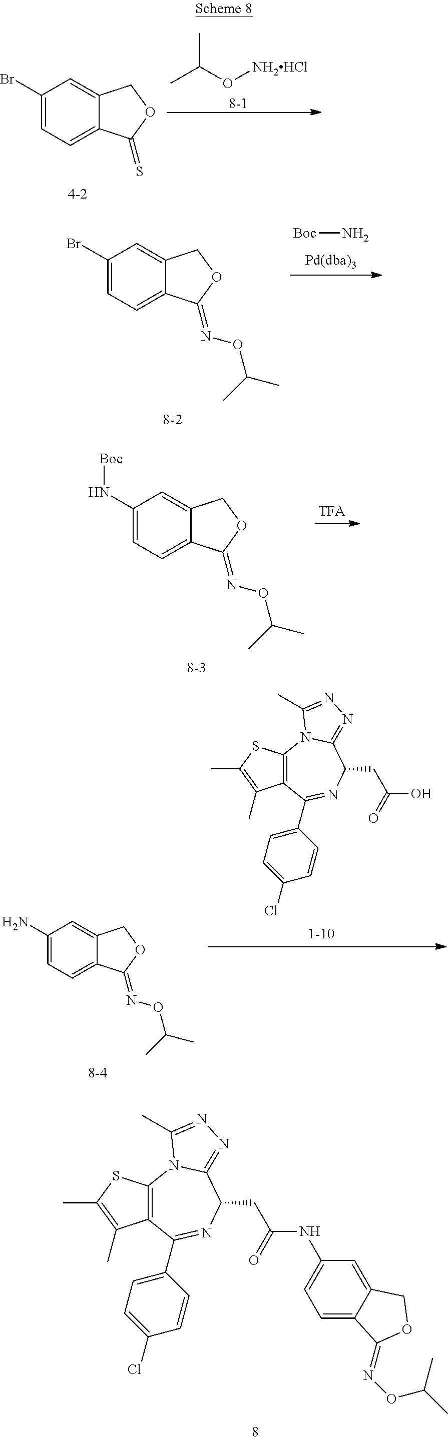

### Example 8

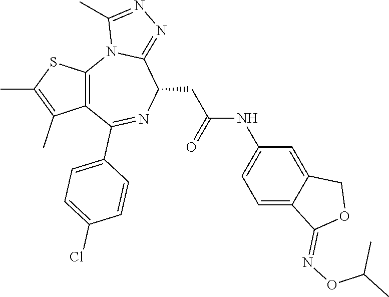 [SMILES: Cc1sc2c(c1C)C(c1ccc(Cl)cc1)=N[C@@H](CC(=O)Nc1ccc3c(c1)CO/C3=N\OC(C)C)c1nnc(C)n1-2]

### Synthesis of Compound 8-2

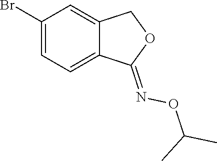

To a reaction flask were added Compound 4-2 (650 mg, 2.84 mmol, 1 eq), EtOH (6.5 mL), Compound 8-1 (411.52 mg, 3.69 mmol, 1.3 eq), and AcONa (698.22 mg, 8.51 mmol, 3 eq). The reaction was stirred at 25° C. for 15 hours. TLC showed that the starting points disappeared and a new point appeared. The system was filtered, and the filtrate was collected and concentrated to dryness under reduced pressure. Water (30 mL) was added to the residue. The mixture was extracted with dichloromethane (10 mL*5). The obtained mixture was extracted. The organic phase was collected and concentrated under reduced pressure. The residue was mixed with silica gel and purified by column chromatography to afford Compound 8-2. LCMS(ESI) m/z: 270.0, 272.0 (M+1, M+3).

### Synthesis of Compound 8-3

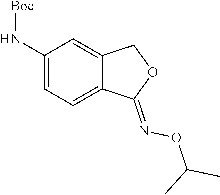 [SMILES: CC(C)O/N=C1\OCc2cc(NC(=O)OC(C)(C)C)ccc21]

To a reaction flask were added Compound 8-2 (535 mg, 1.98 mmol, 1 eq), tert-butyl carbamate (696.05 mg, 5.94 mmol, 3 eq), Cs₂CO₃ (1.61 g, 4.95 mmol, 2.5 eq), Xantphos (114.60 mg, 198.06 μmol, 0.1 eq), and toluene (5.5 mL). The system was purged three times with nitrogen, and then Pd₂(dba)₃ (181.37 mg, 198.06 μmol, 0.1 eq) was added. The system was purged three times with nitrogen again. The reaction was stirred at 120° C. for 15 hours. LCMS showed that the starting materials were completely consumed and the target product was produced. The reaction solution was filtered, and the filtrate was collected. The filtrate was concentrated under reduced pressure. The residue was mixed with silica gel and purified by column chromatography to afford Compound 8-3. LCMS(ESI) m/z: 307.1 (M+1).

### Synthesis of Compound 8-4

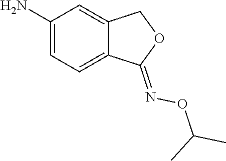

To a reaction flask were added Compound 8-3 (470.00 mg, 1.53 mmol, 1 eq), TFA (10 mL), and dichloromethane (10 mL). The reaction was stirred at 25° C. for 2 hours. TLC showed that the starting points disappeared and a new point appeared. To the system was added a saturated aqueous sodium bicarbonate solution until pH reached 8. The mixture was extracted with dichloromethane (10 mL*5). The organic phase was collected and concentrated to dryness under reduced pressure to afford Compound 8-4, which was directly used for the next step.

### Synthesis of Compound 8

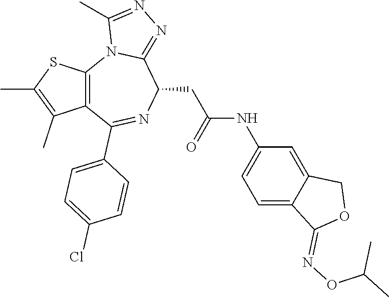 [SMILES: Cc1sc2c(c1C)C(c1ccc(Cl)cc1)=N[C@@H](CC(=O)Nc1ccc3c(c1)CO/C3=N\OC(C)C)c1nnc(C)n1-2]

To a reaction flask were added Compound 8-4 (66.88 mg, 324.29 μmol, 1.3 eq), Compound 1-10 (100 mg, 249.45 μmol, 1 eq), dichloromethane (6 mL), DMAP (3.05 mg, 24.95 μmol, 0.1 eq), DIPEA (41.91 mg, 324.29 μmol, 56.48 μL, 1.3 eq), and T₃P (111.12 mg, 349.23 μmol, 103.85 μL, 1.4 eq, 50% solution in ethyl acetate). The reaction was stirred at 25° C. for 2 hours. LCMS showed the disappearance of starting material peaks and the appearance of target product peaks. Water (40 mL) was added to the system. The mixture was extracted with dichloromethane (10 mL*5). The organic phase was collected, concentrated under reduced pressure to dryness. The residue was purified by preparative HPLC (basic) to afford Compound 8.

¹HNMR (400 MHz, CDCl3) δ=9.36 (s, 1H), 7.86 (s, 1H), 7.60-7.58 (m, 1H), 7.44-7.42 (m, 2H), 7.37-7.33 (m, 3H), 5.38 (s, 2H), 4.62-4.60 (m, 1H), 4.34-4.31 (m, 1H), 3.81-3.78 (m, 1H), 3.52-3.48 (m, 1H), 2.69 (s, 3H), 2.42 (s, 3H), 1.70 (s, 3H), 1.34 (d, J=6.3 Hz, 6H). LCMS(ESI) m/z: 589.3 (M+1).

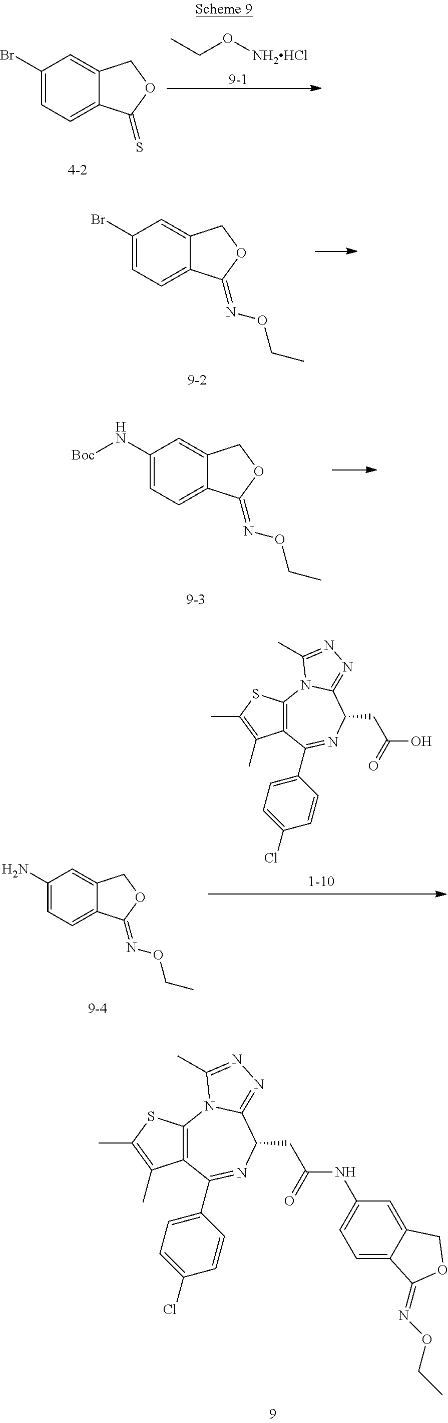

### Example 9

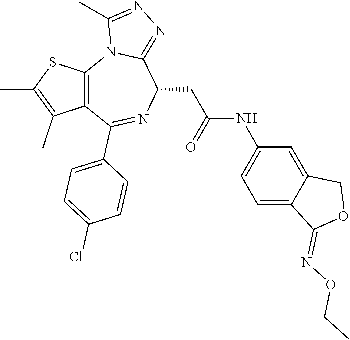 [SMILES: CCO/N=C1\OCc2cc(NC(=O)C[C@@H]3N=C(c4ccc(Cl)cc4)c4c(sc(C)c4C)-n4c(C)nnc43)ccc21]

### Synthesis of Compound 9-2

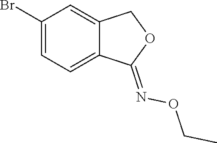

Compound 9-1 (1.00 g, 10.25 mmol, 2.35 eq) and NaOAc (895.16 mg, 10.91 mmol, 2.5 eq) were added to a solution of Compound 4-2 (1 g, 4.37 mmol, 1 eq) in EtOH (10 mL), and the reaction was stirred at 90° C. for 20 hours. LCMS showed that the starting materials were completely consumed and the target product was produced. The reaction was stopped. The reaction solution was cooled to room temperature and concentrated. The crude product obtained was dissolved in dichloromethane (50 mL), and washed with water (50 mL*2). The organic phase was dried over anhydrous sodium sulfate, and filtered. The filtrate was concentrated to afford the product. The obtained crude product was purified by column chromatography to afford Compound 9-2.

¹HNMR (400 MHz, CDCl3) δ=7.52-7.57 (m, 3H), 5.44 (s, 2H), 4.19 (q, J=7.2 Hz, 2H), 1.38 (t, J=7.2 Hz, 3H). LCMS(ESI) m/z: 255.5, 257.5 (M+1, M+3).

### Synthesis of Compound 9-3

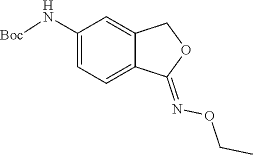 [SMILES: CCO/N=C1\OCc2cc(NC(=O)OC(C)(C)C)ccc21]

To a solution of Compound 9-2 (200 mg, 780.96 μmol, 1 eq) and tert-butyl carbamate (112.28 mg, 958.46 μmol, 1.23 eq) in anhydrous 1,4-dioxane (5 mL) were successively added 2-dicyclohexylphosphino-2′,4′,6′-triisopropylbiphenyl (44.21 mg, 92.74 μmol, 1.19 e-1 eq), Cs₂CO₃ (525.93 mg, 1.61 mmol, 2.07 eq) and Pd(dba)₂ (44.21 mg, 76.89 μmol, 9.85 e-2 eq). The system was purged three times with nitrogen, and then the mixture was reacted in a microwave synthesizer at 120° C. for 0.5 h. LCMS showed that the starting materials were completely consumed and the target product was produced. The reaction was stopped. The reaction solution was diluted with ethyl acetate (10 mL), washed successively with water (10 mL) and saturated saline (10 mL), dried over anhydrous sodium sulfate, and filtered. The filtrate was concentrated under reduced pressure to dryness. The residue was purified by column chromatography to afford Compound 9-3. LCMS(ESI) m/z: 292.9 (M+1).

### Synthesis of Compound 9-4

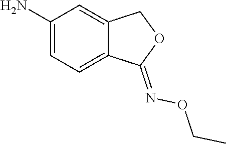

TFA (385.00 mg, 3.38 mmol, 250 μL, 9.87 eq) was added to a solution of Compound 9-3 (0.1 g, 342.08 μmol, 1 eq) in dichloromethane (1 mL). The reaction was stirred at room temperature (25° C.) for 1 hour. LCMS showed that the starting materials were completely consumed and the target product was produced. The reaction was stopped. The reaction solution was diluted with dichloromethane (10 mL) and neutralized with a saturated aqueous sodium bicarbonate solution to a pH of 7. The separated organic phase was washed with saturated saline (10 mL), dried over anhydrous sodium sulfate, and filtered. The filtrate was concentrated to dryness under reduced pressure to afford Compound 9-4. LCMS(ESI) m/z: 192.7 (M+1).

### Synthesis of Compound 9

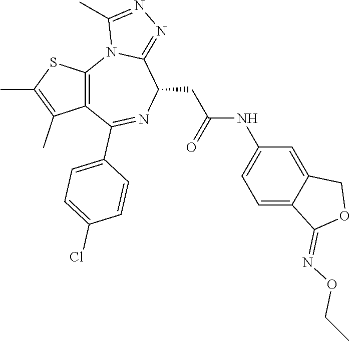 [SMILES: CCO/N=C1\OCc2cc(NC(=O)C[C@@H]3N=C(c4ccc(Cl)cc4)c4c(sc(C)c4C)-n4c(C)nnc43)ccc21]

To a solution of Compound 9-4 (35 mg, 182.09 μmol, 1 eq), Compound 1-10 (90 mg, 224.51 μmol, 1.23 eq) and DMAP (2.22 mg, 18.21 μmol, 0.1 eq) in anhydrous dichloromethane (2 mL) were successively added DIPEA (74.20 mg, 574.11 μmol, 100 μL, 3.15 eq) and T₃P (181.90 mg, 285.84 μmol, 170 μL, 50% purity, 1.57 eq). The reaction was stirred at room temperature (25° C.) for 2 hours. LCMS showed that the starting materials were completely consumed and the target product was produced. The reaction was stopped. The reaction solution was diluted with dichloromethane (10 mL) and washed with water (10 mL). The organic phase was dried over anhydrous sodium sulfate and filtered. The filtrate was concentrated to dryness under reduced pressure. The resulting crude product was purified by preparative HPLC (basic) to afford Compound 9.

¹HNMR (400 MHz, CDCl3) δ=9.61 (s, 1H), 7.83 (s, 1H), 7.56 (d, J=8.4 Hz, 1H), 7.31-7.42 (m, 5H), 5.36 (s, 2H), 4.62-4.65 (m, 1H), 4.15 (q, J=7.2 Hz, 2H), 3.81-3.84 (m, 1H), 3.50-3.54 (m, 1H), 2.69 (s, 3H), 2.41 (s, 3H), 1.69 (s, 3H), 1.35 (t, J=7.2 Hz, 2H). LCMS(ESI) m/z: 575.3 (M+1).

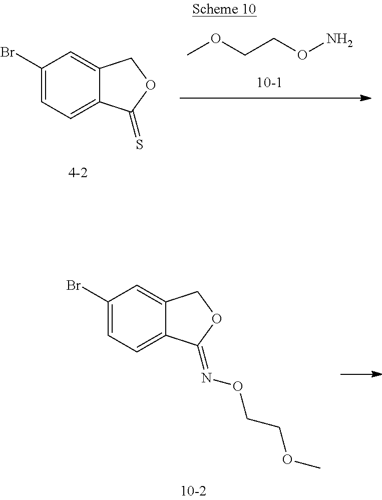

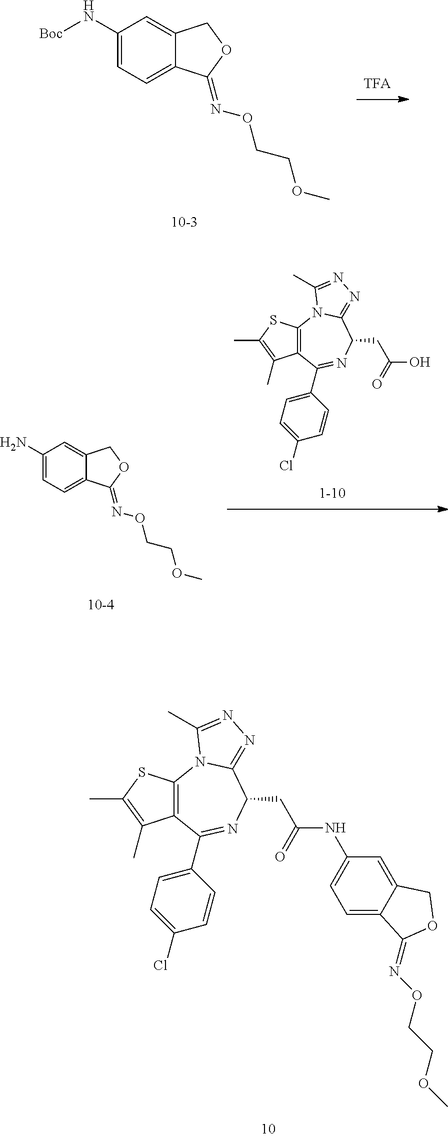

### Example 10

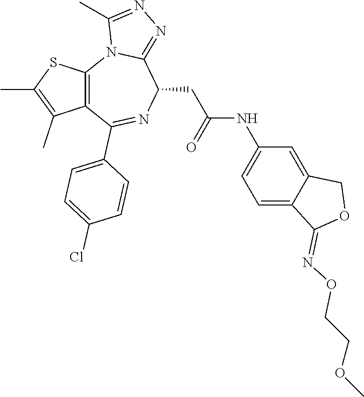

### Synthesis of Compound 10-2

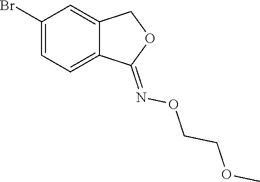 [SMILES: COCCO/N=C1\OCc2cc(Br)ccc21]

Compound 10-1 (200.00 mg, 2.20 mmol, 2.51 eq) and NaOAc (71 mg, 865.50 μmol, 9.91 e-1 eq) were added to a solution of Compound 4-2 (0.2 g, 873.01 μmol, 1 eq) in EtOH (2 mL) and the reaction was stirred at 90° C. for 20 hours. LCMS showed that the starting materials were completely consumed and the target product was produced. The reaction was stopped. The reaction solution was cooled to room temperature and concentrated to afford a crude product. The crude product was dissolved in dichloromethane (10 mL) and washed with water (5 mL*2). The organic phase was dried over anhydrous sodium sulfate, and filtered. The filtrate was concentrated to afford a crude product. The crude product was purified by column chromatography to afford Compound 10-2. LCMS(ESI) m/z: 285.7, 287.7 (M+1, M+3).

### Synthesis of Compound 10-3

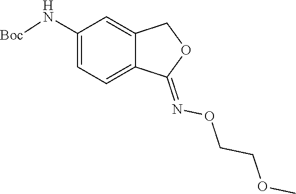 [SMILES: COCCO/N=C1\OCc2cc(NC(=O)OC(C)(C)C)ccc21]

To a solution of Compound 10-2 (80 mg, 279.60 μmol, 1 eq) and tert-butyl carbamate (60 mg, 512.18 μmol, 1.83 eq) in anhydrous 1,4-dioxane (1 mL) were successively added 2-dicyclohexylphosphino-2′,4′,6′-triisopropylbiphenyl (16.00 mg, 33.56 μmol, 0.12 eq), Cs₂CO₃ (200.00 mg, 613.84 μmol, 2.20 eq) and Pd(dba)₂ (16.00 mg, 27.83 μmol, 9.95 e-2 eq). The system was purged three times with nitrogen, and then the mixture was reacted in a microwave synthesizer at 120° C. for 0.5 h. TLC showed that the starting materials were almost completely consumed and the product was mainly produced. The reaction was stopped. The reaction solution was directly filtered, and the filtrate was concentrated under reduced pressure to dryness. The residue was purified by preparative TLC to afford compound 10-3. LCMS(ESI) m/z: 323.1 (M+1).

### Synthesis of Compound 10-4

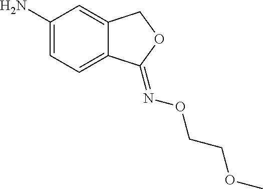 [SMILES: COCCO/N=C1\OCc2cc(N)ccc21]

TFA (308.00 mg, 2.70 mmol, 200 μL, 10.88 eq) was added to a solution of Compound 10-3 (80 mg, 248.17 μmol, 1 eq) in dichloromethane (1 mL), and the reaction was stirred at room temperature (20° C.) for 1 hour. LCMS showed that the starting materials were completely consumed and the target product was produced. The reaction was stopped. The reaction solution was diluted with dichloromethane (10 mL) and neutralized with a saturated aqueous sodium bicarbonate solution to a pH of 7. The separated organic phase was washed with saturated saline (10 mL), dried over anhydrous sodium sulfate, and filtered. The filtrate was concentrated under reduced pressure to dryness to afford Compound 10-4. LCMS(ESI) m/z: 222.6 (M+1).

### Synthesis of Compound 10

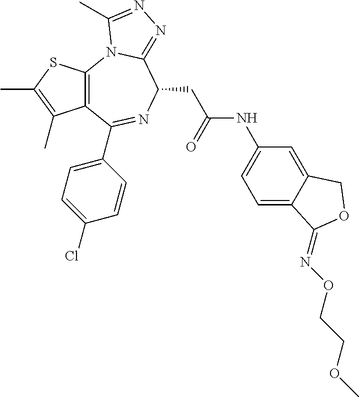

To a solution of Compound 10-4 (50 mg, 224.98 μmol, 1 eq), Compound 1-10 (130 mg, 324.29 μmol, 1.44 eq) and DMAP (3 mg, 24.56 μmol, 1.09 e-1 eq) in anhydrous dichloromethane (2 mL) were successively added DIPEA (111.30 mg, 861.19 μmol, 150 μL, 3.83 eq) and T₃P (267.50 mg, 420.36 μmol, 250 μL, 50% purity, 1.87 eq). The reaction was stirred at room temperature (19° C.) for 2 hours. LCMS showed that the starting materials were completely consumed and the target product was produced. The reaction was stopped. The reaction solution was diluted with dichloromethane (10 mL) and washed with water (10 mL). The organic phase was dried over anhydrous sodium sulfate and filtered. The filtrate was concentrated to dryness under reduced pressure. The resulting crude product was purified by preparative HPLC (basic) to afford Compound 10.

¹HNMR (400 MHz, CDCl3) δ=9.87 (s, 1H), 7.80 (s, 1H), 7.52-7.50 (m, 1H), 7.41-7.36 (m, 3H), 7.32-7.26 (m, 2H), 5.32 (s, 2H), 4.70-4.67 (m, 1H), 4.25-4.22 (m, 2H), 3.87-3.84 (m, 1H), 3.70-3.73 (m, 2H), 3.58-3.59 (m, 1H), 3.40 (s, 3H), 2.69 (s, 3H), 2.41 (s, 3H), 1.68 (s, 3H). LCMS(ESI) m/z: 605.3 (M+1).

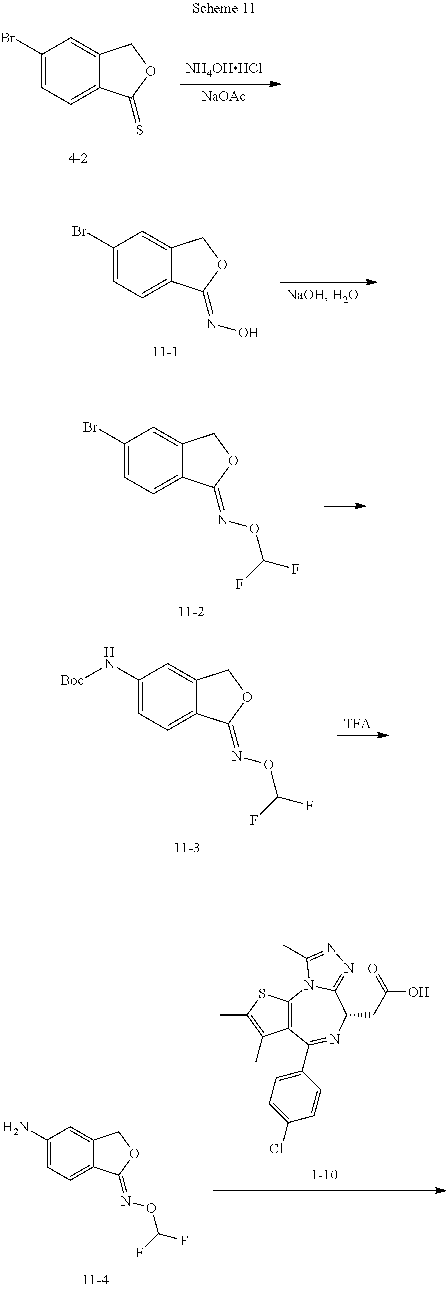

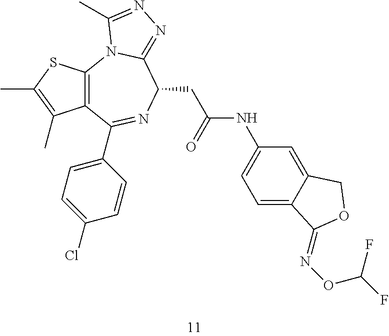 [SMILES: Cc1sc2c(c1C)C(c1ccc(Cl)cc1)=N[C@@H](CC(=O)Nc1ccc3c(c1)CO/C3=N\OC(F)F)c1nnc(C)n1-2]

### Example 11

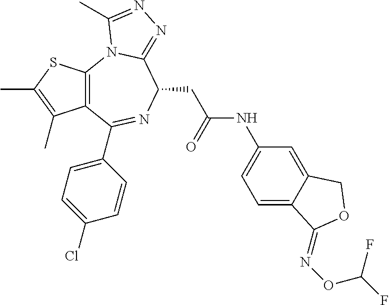 [SMILES: Cc1sc2c(c1C)C(c1ccc(Cl)cc1)=N[C@@H](CC(=O)Nc1ccc3c(c1)CO/C3=N\OC(F)F)c1nnc(C)n1-2]

### Synthesis of Compound 111

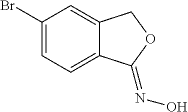 [SMILES: O/N=C1\OCc2cc(Br)ccc21]

To a reaction flask were added EtOH (5 mL), Compound 4-2 (500 mg, 2.18 mmol, 1 eq), NH₂OH.HCl (454.99 mg, 6.55 mmol, 3 eq) and NaOAc (537.10 mg, 6.55 mmol, 3 eq). The reaction was stirred at room temperature (20° C.) for 1 hour. LCMS showed that the starting materials were completely consumed and the target product was produced. The system was filtered, and the filter cake was washed with 10 mL of ethanol. The filtrate was concentrated to dryness, and the residue was then purified by column chromatography to afford Compound 11-1. LCMS(ESI) m/z: 227.9, 229.9 (M+1, M+3).

### Synthesis of Compound 11-2

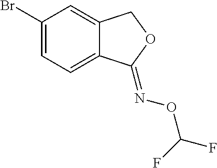 [SMILES: FC(F)O/N=C1\OCc2cc(Br)ccc21]

To a reaction flask were added toluene (35 mL), H₂O (11 mL), and NaOH (4.43 g, 110.83 mmol, 24.07 eq), followed by Compound 11-1 (1.05 g, 4.60 mmol, 1 eq) and TBAI (350.00 mg, 947.57 μmol, 2.06 e-1 eq). The mixture was bubbled with chlorodifluoromethane (398.14 mg, 4.60 mmol, 1 eq) at room temperature (20° C.) for 1 hour. Under chlorodifluoromethane (398.14 mg, 4.60 mmol, 1 eq) balloon of 15 psi, the reaction was then heated in an oil bath at 80° C. for 9 hours. LCMS showed that the starting materials were remained and the target product was produced. To the reaction system was added 200 mL of water. The mixture was extracted twice respectively with 50 mL of ethyl acetate. The organic phases were combined and washed once more with 30 mL of water and once more with 30 mL of saturated saline. The organic phase was then dried over anhydrous sodium sulfate, and filtered. The filtrate was concentrated to dryness. The residue was purified by column chromatography to afford Compound 11-2. LCMS(ESI) m/z: 277.9, 279.9 (M+1, M+3).

### Synthesis of Compound 11-3

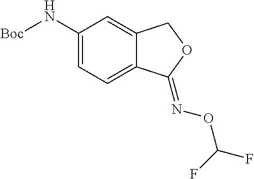 [SMILES: CC(C)(C)OC(=O)Nc1ccc2c(c1)CO/C2=N\OC(F)F]

To a reaction flask were added toluene (2 mL), Compound 11-2 (140.00 mg, 503.51 μmol, 1 eq), tert-butyl carbamate (182.26 mg, 1.56 mmol, 3.09 eq), Cs₂CO₃ (411.60 mg, 1.26 mmol, 2.51 eq), Xantphos (30.80 mg, 53.23 μmol, 1.06 e-1 eq) and Pd₂(dba)₃ (47.60 mg, 51.98 μmol, 1.03 e-1 eq). The system was purged three times with nitrogen, and then the mixture was heated to 120° C. and reacted for 18 hours. LCMS showed that the starting materials were completely consumed and the target product was produced. The reaction solution was concentrated to dryness under reduced pressure, and 20 mL of ethyl acetate and 20 mL of water were added. After extraction, the layers were separated. The organic phase was dried over anhydrous sodium sulfate, and filtered. The filtrate was concentrated to afford a crude product. The crude product was purified by column chromatography to afford Compound 11-3. LCMS(ESI) m/z: 315.1 (M+1).

### Synthesis of Compound 11-4

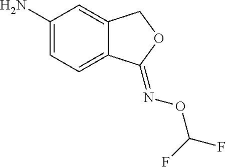 [SMILES: Nc1ccc2c(c1)CO/C2=N\OC(F)F]

To a reaction flask were added dichloromethane (3 mL), Compound 11-3 (110.00 mg, 350.00 μmol, 1 eq) and TFA (1.54 g, 13.51 mmol, 1 mL, 38.59 eq). The mixture was reacted at 15° C. for 1 hour. TLC showed disappearance of starting materials and appearance of a new product. To the reaction system were added 20 mL of saturated sodium bicarbonate solution, and 10 mL of dichloromethane. The layers were separated. The organic phase was dried over anhydrous sodium sulfate, and filtered. The filtrate was concentrated to afford Compound 11-4. LCMS(ESI) m/z: 215.0 (M+1).

### Synthesis of Compound 11

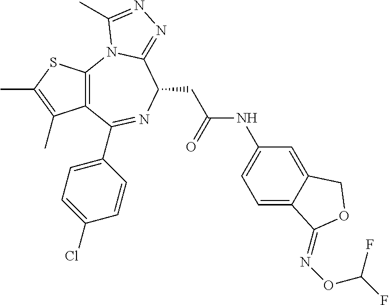 [SMILES: Cc1sc2c(c1C)C(c1ccc(Cl)cc1)=N[C@@H](CC(=O)Nc1ccc3c(c1)CO/C3=N\OC(F)F)c1nnc(C)n1-2]

To a reaction flask were added dichloromethane (2 mL), Compound 1-10 (70 mg, 174.62 μmol, 1 eq) and Compound 11-4 (41.14 mg, 192.08 μmol, 1.1 eq), followed by DMAP (2.1 mg, 17.19 μmol, 9.84 e-2 eq). The system was purged three times with nitrogen, and then DIPEA (32 mg, 247.60 μmol, 43.13 μL, 1.42 eq) and T₃P (167 mg, 262.43 μmol, 156.07 μL, 50% solution in ethyl acetate, 1.5 eq) were added to the reaction. The mixture was reacted at room temperature (20° C.) for 2 hours. LCMS showed disappearance of the starting materials and production of the target product. To the reaction system was added 20 mL of water, followed by 20 mL of dichloromethane. The layers were separated. The aqueous phase was further extracted with 10 mL of dichloromethane. The organic phases were combined, dried over anhydrous sodium sulfate, and filtrated. The filtrate was concentrated to afford a crude product. The crude product was purified by preparative HPLC (basic) to afford Compound 11.

¹HNMR (400 MHz, CDCl3) δ=10.33 (s, 1H), 7.80 (s, 1H), 7.49-7.47 (m, 1H), 7.41-7.39 (m, 3H), 7.31-7.29 (m, 1H), 6.82-6.31 (m, 1H), 5.33 (s, 2H), 4.77-4.74 (m, 1H), 3.99-3.98 (m, 1H), 3.59-3.54 (m, 1H), 2.72 (s, 3H), 2.44 (s, 3H), 1.71 (s, 3H). LCMS(ESI) m/z: 597 (M+1).

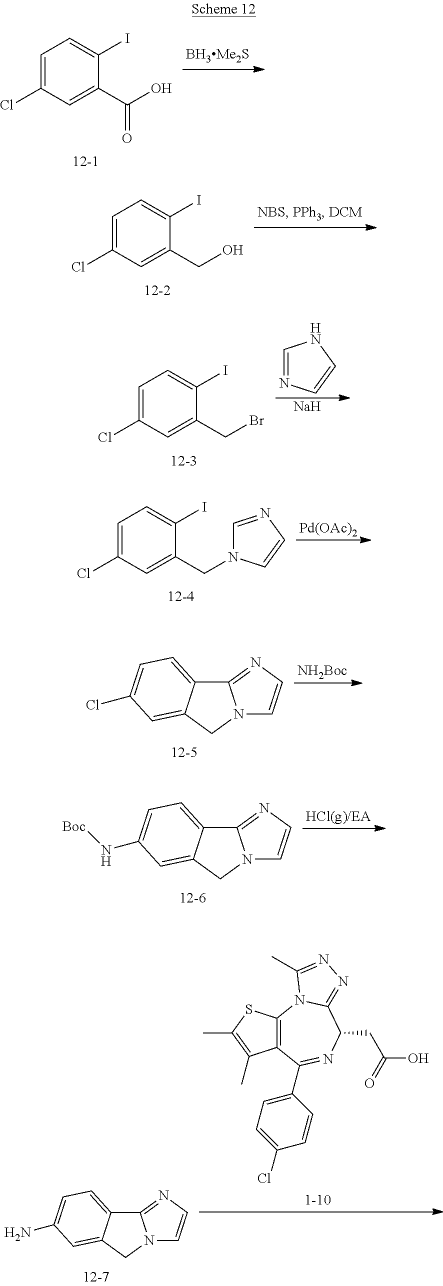

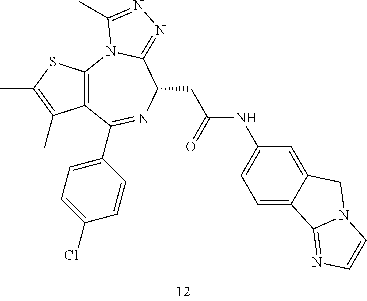 [SMILES: Cc1sc2c(c1C)C(c1ccc(Cl)cc1)=N[C@@H](CC(=O)Nc1ccc3c(c1)Cn1ccnc1-3)c1nnc(C)n1-2]

### Example 12

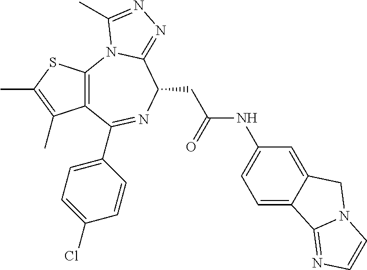 [SMILES: Cc1sc2c(c1C)C(c1ccc(Cl)cc1)=N[C@@H](CC(=O)Nc1ccc3c(c1)Cn1ccnc1-3)c1nnc(C)n1-2]

### Synthesis of Compound 12-2

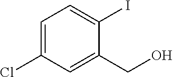

To a 100 mL three-necked flask were added Compound 12-1 (5 g, 17.70 mmol, 1 eq) and THF (50 mL). The reaction was cooled to 0° C. Borane dimethyl sulfide (10M, 3.54 mL, 2 eq) was added to the reaction at 0-5° C. The reaction was slowly warmed to room temperature (20° C.) and stirred for 12 hours. The reaction was then heated to 50° C. and stirred for 6 hours. TLC showed that the starting materials were completely consumed and one main point appeared. The reaction solution was cooled to 0 to 5° C., and anhydrous methanol (50 mL) was slowly added to the solution at this temperature with bubbles formed. After the dropwise addition, the mixture was heated to 80° C. in an oil bath, and refluxed for 1 hour. The reaction was fully quenched, and the mixture was concentrated. The residue was purified by column chromatography to afford Compound 12-2.

¹HNMR (400 MHz, CDCl3) δ=7.73 (d, J=8.4 Hz, 1H), 7.49 (d, J=2.4 Hz, 1H), 7.02 (d, J=8.4, 2.4 Hz, 1H), 4.65 (d, J=6.0 Hz, 2H), 2.02 (t, J=6.0 Hz, 1H).

### Synthesis of Compound 12-3

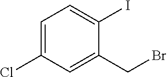

To a reaction flask were added dichloromethane (75 mL), NBS (12.43 g, 69.84 mmol, 2.5 eq), and PPh₃ (14.65 g, 55.87 mmol, 2 eq) at 0° C. Compound 12-2 (7.5 g, 27.94 mmol, 1 eq) was added to the reaction at 0° C. The reaction was stirred at room temperature (20° C.) for 16 hours. LCMS and TLC showed that the starting material points disappeared and a new point appeared. 100 mL of water was added to the system. The layers were separated. The organic phase was washed with 100 mL of saturated saline, dried over anhydrous sodium sulfate, and filtered. The filtrate was concentrated under reduced pressure. The residue was purified by column chromatography to afford Compound 12-3.

¹HNMR (400 MHz, CDCl3) δ=7.77 (d, J=8.4 Hz, 1H), 7.47 (d, J=2.6 Hz, 1H), 6.99 (dd, J=2.5, 8.4 Hz, 1H), 4.53 (s, 2H).

### Synthesis of Compound 12-4

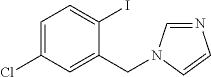 [SMILES: Clc1ccc(I)c(Cn2ccnc2)c1]

To a reaction flask was added Compound 12-3 (5 g, 15.09 mmol, 1 eq) in THF (50 mL), and the mixture was cooled to 0° C. NaH (663.90 mg, 16.60 mmol, 60% purity, 1.1 eq) was added to the reaction in batches at 0-5° C. and the mixture was reacted at 0-5° C. for 0.5 h. Imidazole (1.08 g, 15.84 mmol, 1.05 eq) was then added to the reaction. The reaction was slowly warmed to room temperature (20° C.) and further stirred for 12 hours. TLC showed that the starting material points disappeared and a main point appeared. Saturated ammonium chloride (100 mL) was added to the reaction solution. The layers were separated. The organic phase was extracted with ethyl acetate (100 mL*2). The organic phases were combined, washed with saturated saline (50 mL), dried over anhydrous sodium sulfate, and filtered. The filtrate was concentrated and the residue was purified by column chromatography to afford Compound 12-4.

¹HNMR (400 MHz, CDCl3) δ=7.81 (d, J=8.4 Hz, 1H), 7.59 (s, 1H), 7.16 (s, 1H), 7.05 (dd, J=8.4, 2.4 Hz, 1H), 6.95 (s, 1H), 6.79 (s, 1H), 5.13 (s, 2H). LCMS(ESI) m/z: 318.9 (M+1).

### Synthesis of Compound 12-5

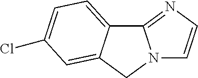 [SMILES: Clc1ccc2c(c1)Cn1ccnc1-2]

To a microwave tube were added Compound 12-4 (1 g, 3.14 mmol, 1 eq) and DMF (12 mL). Pd(OAc)₂ (70.48 mg, 313.93 μmol, 0.1 eq), tris(2-methylphenyl)phosphine (286.65 mg, 941.80 μmol, 0.3 eq) and AcOK (616.19 mg, 6.28 mmol, 2 eq) were successively added to the reaction. The reaction was heated with microwave at 150° C. for 1 hour (0 bar). LCMS showed that the starting materials were completely consumed and the target product was produced. The reaction solution was cooled to room temperature. Water (100 mL) was added to the reaction, and the mixture was stirred for 2 minutes. The mixture was extracted with ethyl acetate (100 mL*3). The combined organic phase was washed with saturated saline (100 mL), dried over anhydrous sodium sulfate, and filtered. The filtrate was concentrated under reduced pressure to afford a crude product. The crude product was purified by column chromatography to afford Compound 12-5.

¹HNMR (400 MHz, CDCl3) δ=7.74 (s, 1H), 7.49 (d, J=8.0 Hz, 1H), 7.40-7.36 (m, 2H), 7.20 (s, 1H), 5.02 (s, 2H). LCMS(ESI) m/z: 191.0 (M+1).

### Synthesis of Compound 12-6

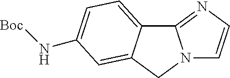 [SMILES: CC(C)(C)OC(=O)Nc1ccc2c(c1)Cn1ccnc1-2]

To a reaction flask were added degassed 1,4-dioxane (8.8 mL) and Compound 12-5 (880 mg, 4.62 mmol, 1 eq). Tert-butyl carbamate (1.62 g, 13.85 mmol, 3 eq), Cs₂CO₃ (3.76 g, 11.54 mmol, 2.5 eq), 2-dicyclohexylphosphino-2′,4′,6′-triisopropylbiphenyl (220.07 mg, 461.63 μmol, 0.1 eq) and Pd(dba)₂ (530.88 mg, 923.26 μmol, 0.2 eq) were successively added to the reaction. The reaction was heated to 105° C. and stirred for 12 hours. LCMS showed that the starting materials were completely consumed and the target product was produced. The reaction solution was cooled to room temperature and concentrated under reduced pressure. Ethyl acetate (20 mL) and H₂O (20 mL) were added, and insolubles were filtered off through a pad of celite. The layers were separated, and the aqueous phase was extracted with ethyl acetate (20 mL*2). The combined organic phase was washed with saturated saline (10 mL), dried over anhydrous sodium sulfate, and filtered. The filtrate was concentrated to afford Compound 12-6. LCMS(ESI) m/z: 272.1 (M+1).

### Synthesis of Compound 12-7

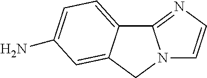 [SMILES: Nc1ccc2c(c1)Cn1ccnc1-2]

To a reaction flask were added Compound 12-6 (0.9 g, 3.32 mmol, 1 eq) and ethyl acetate (0.5 mL). After the starting materials were dissolved, the system was cooled to 0-5° C. HCl (gas)/ethyl acetate (4M, 14.93 mL, 18 eq) was added to the system. After the addition, the reaction was slowly warmed to room temperature (20° C.) and further stirred for 12 hours. TLC showed that the starting materials were completely consumed and the target product was produced. The reaction solution was washed with water (20 mL*2). The combined aqueous phase was cooled to 0-5° C., and saturated aqueous solution of sodium bicarbonate was added to adjust pH to ˜8. The aqueous phase was extracted with ethyl acetate (30 mL*3) and the combined organic phase was washed with saturated saline (10 mL). The organic phase was dried over anhydrous sodium sulfate, and filtered. The filtrate was concentrated. The residue was purified by column chromatography to afford Compound 12-7.

¹HNMR (400 MHz, CDCl3) δ=7.65 (s, 1H), 7.35 (d, J=8.0 Hz, 1H), 7.02 (s, 1H), 6.73 (s, 1H), 6.70-6.68 (m, 1H), 4.92 (s, 2H), 3.76 (brs, 2H). LCMS(ESI) m/z: 172.1 (M+1).

### Synthesis of Compound 12

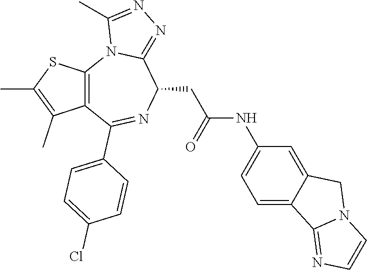 [SMILES: Cc1sc2c(c1C)C(c1ccc(Cl)cc1)=N[C@@H](CC(=O)Nc1ccc3c(c1)Cn1ccnc1-3)c1nnc(C)n1-2]

To a reaction flask were added Compound 1-10 (18.45 mg, 107.76 μmol, 1.08 eq) and dichloromethane (2 mL). Compound 12-7 (40 mg, 99.78 μmol, 1 eq) and DMAP (1.22 mg, 9.98 μmol, 0.1 eq) were then added to the reaction. The system was purged 3 times with nitrogen. DIPEA (17.80 mg, 137.70 μmol, 23.98 μL, 1.38 eq) and T₃P (90.16 mg, 141.69 μmol, 84.27 μL, 50% purity, 1.42 eq) were then added to the reaction. The reaction was stirred at room temperature (20° C.) for 2 hours. LCMS showed that the starting materials were completely consumed and the target product was produced. To the reaction solution were added dichloromethane (10 mL) and H₂O (10 mL), and the mixture was stirred for 2 min. The layers were separated, and the aqueous phase was extracted with dichloromethane (5 mL*2). The combined organic phase was washed with saturated saline (5 mL), dried over anhydrous sodium sulfate, and filtered. The filtrate was concentrated to afford a crude product. The crude product was purified by preparative HPLC (basic) to afford Compound 12.

¹HNMR (400 MHz, CDCl3) δ=9.45 (s, 1H), 7.85 (s, 1H), 7.63 (s, 1H), 7.44-7.40 (m, 3H), 7.35-7.31 (m, 3H), 7.10 (s, 1H), 4.92-4.79 (m, 2H), 4.70-4.66 (m, 1H), 3.93-3.87 (m, 1H), 3.53-3.49 (m, 1H), 2.72 (s, 3H), 2.43 (s, 3H), 1.71 (s, 3H). LCMS(ESI) m/z: 554.2 (M+1).

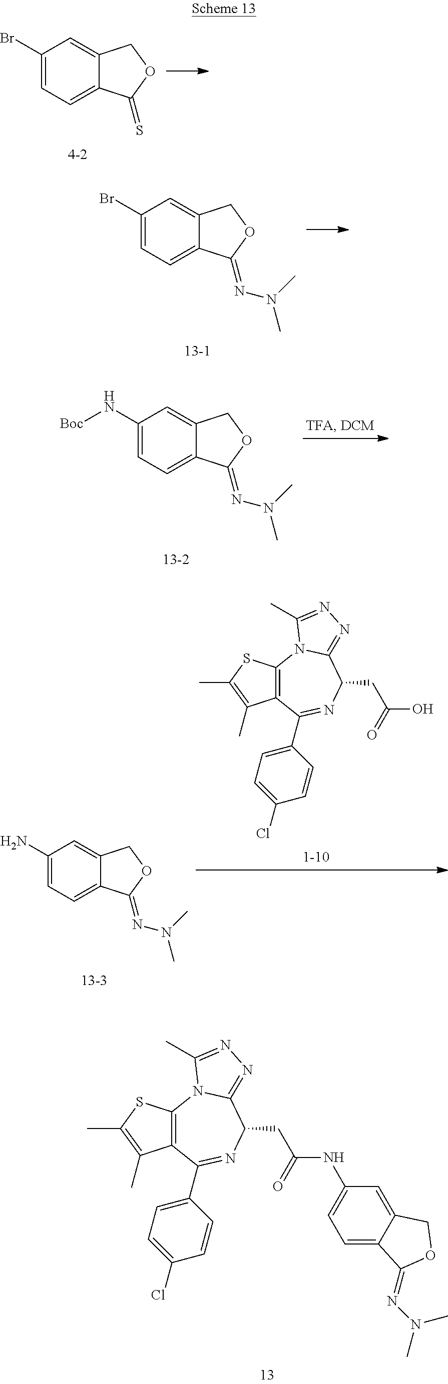

### Example 13

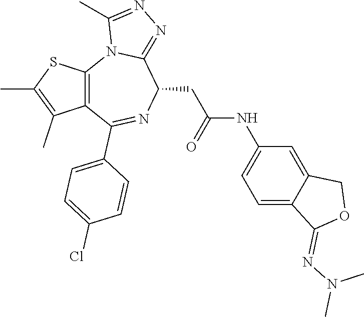

### Synthesis of Compound 13-1

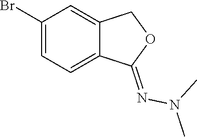

1,1-dimethylhydrazine hydrochloride (720.00 mg, 7.46 mmol, 909.09 μL, 1.42 eq) and NaOAc (1.3 g, 15.85 mmol, 3.03 eq) were added to a solution of Compound 4-2 (3.0 g, 5.24 mmol, 1 eq) in EtOH (30 mL), and the reaction was stirred at 90° C. for 20 hours. LCMS showed complete consumption of the starting materials and production of the product (23.11%). The reaction was stopped. The reaction solution was cooled to room temperature, and water (20 mL) and dichloromethane (20 mL) were added. The mixture was stirred at room temperature for 5 minutes, and then allowed to stand for layer separation. The organic phase was separated, washed with saturated saline (20 mL), dried over anhydrous sodium sulfate, and filtered. The filtrate was concentrated under reduced pressure to dryness. The obtained crude product was purified by column chromatography to afford Compound 13-1. LCMS(ESI) m/z: 254.5, 256.5 (M+1, M+3).

### Synthesis of Compound 13-2

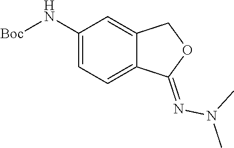 [SMILES: CN(C)/N=C1\OCc2cc(NC(=O)OC(C)(C)C)ccc21]

To a solution of Compound 13-1 (85 mg, 333.19 μmol, 1 eq) and tert-butyl carbamate (80 mg, 682.91 μmol, 2.05 eq) in anhydrous 1,4-dioxane (2 mL) were successively added 2-dicyclohexylphosphino-2′,4′,6′-triisopropylbiphenyl (17.00 mg, 35.66 μmol, 1.07 e-1 eq), Cs₂CO₃ (600.00 mg, 1.84 mmol, 5.53 eq) and Pd(dba)₂ (17.00 mg, 29.56 μmol, 8.87 e-2 eq). The system was purged three times with nitrogen, and then the mixture was reacted in a microwave synthesizer at 120° C. for 1 hour. LCMS showed that the starting materials were completely consumed and the target product was produced. The reaction solution was purified by preparative TLC to afford Compound 13-2. LCMS(ESI) m/z: 291.9 (M+1).

### Synthesis of Compound 13-3

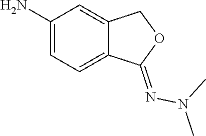

TFA (308.00 mg, 2.70 mmol, 200 μL, 11.24 eq) was added to a solution of Compound 13-2 (70 mg, 240.26 μmol, 1 eq) in dichloromethane (1 mL), and the reaction was stirred at room temperature (20° C.) for 1 hour. LCMS showed that the starting materials were completely consumed and the target product was produced. The reaction solution was diluted with dichloromethane (10 mL) and then neutralized with a saturated aqueous sodium bicarbonate solution to a pH of 7. The separated organic phase was washed with saturated saline (10 mL), dried over anhydrous sodium sulfate, and filtered. The filtrate was concentrated to dryness under reduced pressure to afford Compound 13-3. LCMS(ESI) m/z: 191.9 (M+1).

### Synthesis of Compound 13

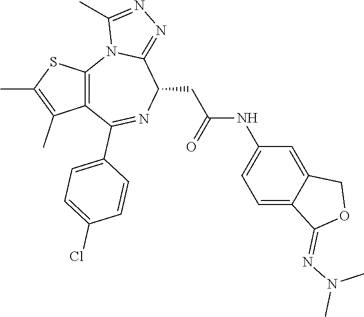

To a solution of Compound 13-3 (25 mg, 130.73 μmol, 1 eq), Compound 1-10 (80 mg, 199.56 μmol, 1.53 eq) and DMAP (2 mg, 16.37 μmol, 1.25 e-1 eq) in anhydrous dichloromethane (2 mL) were successively added DIPEA (59.36 mg, 459.30 μmol, 80 μL, 3.51 eq) and T₃P (149.80 mg, 235.40 μmol, 140 μL, 50% solution in ethyl acetate, 1.80 eq). The reaction was stirred at room temperature (19° C.) for 2 hours. LCMS showed that the starting materials were completely consumed and the target product was produced. The reaction solution was diluted with dichloromethane (10 mL) and washed with water (10 mL). The organic phase was dried over anhydrous sodium sulfate and filtered. The filtrate was concentrated to dryness under reduced pressure. The resulting crude product was purified by preparative HPLC (basic) to afford Compound 13.

¹HNMR (400 MHz, CDCl3) δ=9.61 (s, 1H), 7.83 (s, 1H), 7.64-7.62 (m, 1H), 7.42-7.40 (m, 2H), 7.35-7.33 (m, 3H), 5.32 (s, 2H), 4.66-4.63 (m, 1H), 3.87-3.81 (m, 1H), 3.55-3.51 (m, 1H), 2.69 (s, 3H), 2.68 (s, 6H), 2.41 (s, 3H), 1.69 (s, 3H). LCMS(ESI) m/z: 573.9 (M+1).

### BIOLOGICAL ASSAY

### Assay Example 1: Assay of BRD4 Biochemical Activity of the Compounds of the Present Disclosure

1. Assay Preparation

Test 1: IC₅₀ characterization of the compounds at 10 concentrations.

The compounds were evaluated for IC₅₀ against two BRD (BRD4-1, BRD4-2) at 10 concentrations, single well.

2. Test Condition

Buffer composition for BRD: 50 mM HEPES-HCl, pH7.5, 100 mM NaCl, 0.1% BSA, 0.05% CHAPS, and 1% DMSO.

Ligand: histone H4 peptide (1-21) K5/8/12/16Ac-Biotin

Assay: AlphaScreen binding assay (Ex/Em=680/520-620 nm)

Assay Procedures:

2.1 4×BRD was added into wells of a reaction plate, except for control wells without BRD, in which BRD was replaced with a buffer.

2.2 A solution of the compounds in 100% DMSO was added to the BRD mixture using Acoustic Technology (Echo550, nanoliter level). The mixture was centrifuged, shaken gently at room temperature, and pre-incubated for 30 minutes.

2.3 4× ligand was added, and the mixture was centrifuged and shaken.

2.4 The mixture was shaken gently and incubated at room temperature for 30 minutes.

2.5 4× donor beads were added in the dark, and the mixture was centrifuged and shaken.

2.6 4× acceptor beads were added in the dark, and the mixture was centrifuged and shaken gently in the dark for 60 minutes.

2.7 The Alpha assay was carried out using Enspire (Ex/Em=680/520-620 nm)

Data Analysis:

The signal value of the control well (DMSO) was defined as 100% enzyme activity, and the signal value of the background well (in the buffer, no BRD was added but ligand was added) was defined as 0% enzyme activity (or 100% inhibition).

The percent enzyme activity for each test solution was calculated by Microsoft Excel 2003 or 2007 software using the following equation: Percent enzyme activity={{[signal value]-[background signal value]}/{[signal value of DMSO control well]-[background signal value]}}×100

The four-parameter fitting was performed by GraphPadPrism4 software using the following formula to obtain an IC₅₀ fitting curve:

Y=minimum+(maximum−minimum)/(1+10\hat{ }((Log IC50−X)*HillSplope))

Prism settings: 4-parameter dose-response curve (with variable slope), constrained; minimum=0, maximum=less than 120

The curve fitting was performed when the percentage enzyme activity corresponding to the compounds at the highest concentration was less than 65%.

3. Result

Test 1: IC₅₀ characterization of the compounds at 10 concentrations.

The compounds were tested for IC₅₀ against BRD4-1 and BRD4-2 at 10 concentrations, single well, initial concentration of 10 μM, and 3-fold serial dilution.

IC₅₀ values (molar concentration) were summarized in the table below.

Summary Table: IC₅₀ (M)

TABLE 1
IC₅₀ Results of BRD4 Assay

| | BRD4 (BD1, BD2), |
|---|---|
| Compound | IC₅₀ (nM) |
| 1 | 48.8, 7.4 |
| 2 | 48.2, 6.23 |
| 3 | 27.0, 4.45 |
| 4 | 28.0, 6.6 |
| 5 | 31.2, 4.06 |
| 6 | 63.8, 15.1 |
| 7 | 28.7, 6.06 |
| 8 | 85.7, 15.0 |
| 9 | 43.6, 7.0 |
| 10 | 27.0, 3.56 |
| 11 | 72.2, 3.92 |
| 12 | 18.1, 1.99 |
| 13 | 13.1, 2.26 |

Conclusion: The compounds of the present disclosure all have significant activity of inhibiting BET bromdomain.

### Assay Example 2: Regulation of Cell PD-L1 by the Compounds of the Present Disclosure

### (1) Effect of the Compounds on PD-L1 of MCF7 Cells

Assay Purpose:

The downregulation of the compounds on PD-L1 gene was evaluated by detecting the effect of the compounds on PD-L1 of MCF7 cells through a qPCR test.

Assay Method:

MCF7 cells were stimulated with 5 NM of the compounds and interferon γ, respectively. The cells were cultured for 18 hours, and then the samples were collected and detected by qPCR method. The content of DMSO in the detection reaction was 0.1%.

Reagents:

### Takara PrimeScript™ RT Master Mix Kit-RR036A;

### Thermo Power SYBR™ Green PCR Master Mix Kit-4367659;

### QIAGEN RNeasy Mini Kit-74106.

Compound:

The test compound was dissolved in 100% DMSO and diluted to 10 mM for subsequent use. Interferon γ was diluted with PBS to a final concentration of 100 ng/mL.

Assay Procedures:

To cell samples were respectively added each compound and interferon γ to a final concentration of 5 μM and 100 ng/mL. After incubation with drugs for 18 hours, RNA was extracted from the cells using RNeasy kit and inverted to cDNA using Takara inversion kit. Gene primers and SYBR™ Green reagent were added to the cDNA to detect the relative content of the target gene by qPCR.

Reaction Detection:

The relative abundance of the target gene was obtained by reading the plate with QuantStudio 7 instrument.

The assay results were shown in FIG. 1.

Assay Conclusion:

The compounds of the present disclosure have significant down-regulation effect on PD-L1 gene expression.

### (2) Effect of the Compounds on PD-L1 of MDA-MB-231 Cells

Assay Purpose:

The downregulation of the compounds on PD-L1 gene was evaluated by detecting the effect of the compounds on PD-L1 of MDA-MB-231 cells through a qPCR test.

Assay Method:

MDA-MB-231 cells were stimulated with each compound at 250 nM, and cultured for 18 hours. The samples were collected and detected by qPCR method. The content of DMSO in the detection reaction was 0.1%.

Reagents:

Takara PrimeScript™ RT Master Mix Kit-RR036A

Thermo Power SYBR™ Green PCR Master Mix Kit-4367659

QIAGEN RNeasy Mini Kit-74106.

Compound:

The test compound was dissolved in 100% DMSO and diluted to 10 mM for subsequent use.

Assay Procedures:

Each compound was added to cell samples to a final concentration of 250 nM, respectively. After incubation with drugs for 18 hours, RNA was extracted from the cells using RNeasy kit and inverted to cDNA using Takara inversion kit. Gene primers and SYBR™ Green reagent were added to the cDNA to detect the relative content of the target gene by qPCR.

Reaction Detection:

The relative abundance of the target gene was obtained by reading the plate with QuantStudio 7 instrument.

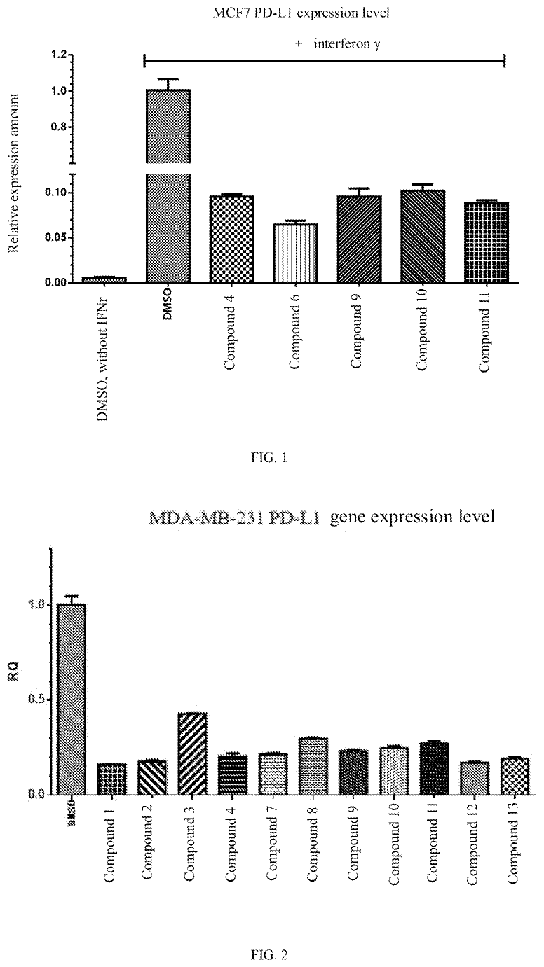

The assay results were shown in FIG. 2.

Assay Conclusion:

The compounds of the present disclosure have efficient down-regulation effect on PD-L1 gene expression.

### Assay Example 3: Pharmacokinetic Studies of the Compounds of the Present Disclosure

1. Abstract

1.1 Male CD-1 mice were used as test animals, and the LC/MS/MS method was used to determine the drug concentration in plasma of mice at different times after intravenous and intragastric administration of the test compounds. The pharmacokinetic behavior of the compounds of the present disclosure in mice was studied, and the pharmacokinetic characteristics of the compounds of the present disclosure were evaluated.

2. Assay Scheme

2.1 Assay Drugs: Test Compounds.

2.2 Assay animals: Eight healthy adult male CD-1 mice, which were divided into four groups (two for each group) according to the principle of similar body weight. The animals were purchased from Shanghai Sippe-Bk Lab Animal Co., Ltd., with animal production license No. of SCXK (Shanghai) 2018-0006.

2.3 Drug Preparation

A proper amount of samples were weighed, to which a solvent was added. The mixture was stirred with ultrasound to a clear state for intravenous administration.

A proper amount of samples were weighed, to which a solvent was added. The mixture was stirred with ultrasound to a clear state for intragastric administration.

2.4 Administration

Eight male CD-1 mice were divided into four groups. After one-night fasting, two groups were administered intravenously, and the remaining two groups were administered intragastrically.

3. Operation

After intravenous administration of the test compounds to male CD-1 mice, 30 L of blood was collected at 0.0833, 0.25, 0.5, 1, 2, 4, 8, and 24 hours, and placed in commercial tubes containing EDTA-K₂. After intragastric administration of the test compounds, 30 L of blood was collected at 0.25, 0.5, 1, 2, 4, 6, 8, and 24 hours, and placed in commercial tubes containing EDTA-K₂. The tubes were centrifuged at 3000 g for 15 minutes to separate plasma and stored at −60° C. Animals were fed 2 hours after the administration.

The LC/MS/MS method was used to determine the content of the test compounds in the plasma of mice after intravenous and intragastric administration. The linear range of the method was 2.00-6000 nmol/L; The plasma samples were analyzed after protein precipitation with acetonitrile.

4. Pharmacokinetic Parameter Results

TABLE 2
Summary of Pharmacokinetic Parameter Data

| | | | | | | | | Area | |
|---|---|---|---|---|---|---|---|---|---|
| | | Drug | | | Apparent | | | under | |
| | | concentration | Peak | Half | volume | Clearance | Area under | curve | |
| | | in | arrival | life | of | rate | curve (0-t) | (0-inf) | bio- |
| Mode of | Dose | blood | time | period | distribution | Cl | AUC₀₋ₗₐₛₜ | AUC_{0-inf} | availability |
| administration | administered | Cmax(nM) | Tmax(h) | T½(h) | Vdss(L/kg) | (mL/min/kg) | (nM · h) | (nM · h) | F(%) |
| Compound | 1 mg/kg | — | — | 2.05 | 1.84 | 10.2 | 2715 | 2910 | — |
| 4 is | | | | | | | | | |
| administered | | | | | | | | | |
| intravenously | | | | | | | | | |
| Compound | 2 mg/kg | 262 | 4.00 | ND | — | — | 1606 | ND | 29.6 |
| 4 is | | | | | | | | | |
| administered | | | | | | | | | |
| intragastrically | | | | | | | | | |
| Compound | 1 mg/kg | — | — | 2.16 | 2.21 | 11.5 | 2260 | 2439 | — |
| 11 is | | | | | | | | | |
| administered | | | | | | | | | |
| intravenously | | | | | | | | | |
| Compound | 2 mg/kg | 115 | 7.00 | ND | — | — | 1311 | ND | 29.0 |
| 11 is | | | | | | | | | |
| administered | | | | | | | | | |
| intragastrically | | | | | | | | | |

“—”: none; ND: not detected.

Assay Conclusion:

The compounds of the present disclosure have short half-life period, extensive distribution outside plasma, and moderate bioavailability.

### Assay Example 4. Study on Anti-Tumor Efficacy in Subject

### (1) In Vivo Efficacy Study of the Compounds of the Present Disclosure in an Animal Transplanted Tumor Model of Colon Cancer Cells in MC38 Mice

1. Assay Design

TABLE 3
Animal grouping and administering scheme of
in vivo efficacy assay

| | | | | Administering | | |
|---|---|---|---|---|---|---|
| | Number | | | volume | | |
| | of | Compound | Dose | parameters | Route of | Administering |
| Group | animals | therapy | (mg/kg) | (l/g) | administration | frequency |
| 1 | 8 | Vehicle | — | 10 | p.o. | BID |
| 2 | 8 | Compound | 20 | 10 | p.o. | BID |
| | | 4 | | | | |
| 3 | 8 | Compound | 50 | 10 | p.o. | BID |
| | | 4 | | | | |
| 4 | 8 | Compound | 20 | 10 | p.o. | BID |
| | | 11 | | | | |
| 5 | 8 | Compound | 50 | 10 | p.o. | BID |
| | | 11 | | | | |

*p.o: oral; BID: twice a day.

2. Assay Materials

2.1 Assay Animals

Species: mice.

Strain: C57BL/6 mice.

Week age and body weight: 6-8 weeks old.

Gender: female.

Supplier: Shanghai slack Laboratory Animal Co., Ltd.

3. Assay Method and Steps

3.1 Cell Culture

Mouse colon cancer MC38 cells (Art. No. HYC3401) were cultured in vitro in single layer under the conditions of DMEM containing 10% fetal bovine serum and 37° C. 5% CO₂ incubator. Trypsin-EDTA was used for routine digestion and passage. When the cells were in exponential growth phase and the saturation was 80%-90%, the cells were collected and counted.

3.2 Tumor Cell Inoculation

Cells were re-suspended in DPBS at a density of 2×10⁵ cells/mL. 0.1 mL DPBS (containing 2×10⁵ MC38 cells) was subcutaneously inoculated into the right back of each mouse. When average tumor volume reached ˜63 mm³, mice were randomly grouped and administered according to tumor volume.

3.3 Tumor Measurement and Assay Indicators

Tumor diameter was measured with vernier caliper three times a week. The calculation formula of tumor volume was as follows: V=0.5×a×b², wherein a and b indicated the long and short diameters of a tumor, respectively.

The anti-tumor effect of the compounds was evaluated using TGI (%) or relative tumor proliferation rate T/C (%). Relative tumor proliferation rate T/C (%)=T_{RTV};/C_{RTV}×100 (T_{RTV}: average RTV for treatment group; C_{RT}V: average RTV for negative control group). According to the results of tumor measurement, relative tumor volume (RTV) was calculated using the formula: RTV=Vₜ/V₀, wherein V₀ is the tumor volume measured when grouped and administered (i.e., D0), Vₜ is the tumor volume at a certain measurement, and the data of T_{RTV} and C_{RTV} on the same day were used.

TGI (%) reflects the rate of tumor growth inhibition. TGI (%)=[(1−(Average tumor volume at the end of administration for a treatment group−Average tumor volume at the beginning of administration for the treatment group))/(Average tumor volume at the end of treatment for a vehicle control group-Average tumor volume at the beginning of treatment for the vehicle control group)]×100.

Tumor weights will be measured at the end of the assay, and T_{weight}/C_{weight} percent will be calculated, wherein T_{weight}/C_{weight} represents tumor weights of the administration group and the vehicle control group, respectively.

3.4 Statistical Analysis

Statistical analysis was performed using SPSS software based on the relative tumor volume and tumor weight at the end of the test. T-test was used for comparison between two groups and one-way ANOVA was used for comparison among three or more groups. If variance was not neat (significant difference was found in F value), Games-Howell method was used for test. P<0.05 was considered a significant difference.

4. Assay Conclusion

The results were shown in Table 4.

TABLE 4

| Group | Dose (mpk) | TGI (%) | P value |
|---|---|---|---|
| Compound 4 | 20 | 85.90 | 0.009 |
| | 50 | 97.66 | 0.005 |
| Compound 11 | 20 | 54.16 | 0.091 |
| | 50 | 84.11 | 0.010 |

Conclusion:

After 18 days of administration observation, compared with the vehicle control group, the compounds of the present disclosure show remarkable tumor inhibition effects at the administration doses. In addition, the compounds of the present disclosure show a dose-dependent effect, and the tumor inhibition effect in the high dose group is better than that in the low dose group. During the administration period, the animals in all dose groups were well tolerated.

### (2) In Vivo Efficacy Study of the Compounds of the Present Disclosure in a PAN02 Tumor Model

Assay Purpose:

The anti-tumor effect of the test compound was investigated on an in vivo tumor model of mouse pancreatic cancer PAN02.

Assay Method:

Female C57BL/6 mice were subcutaneously inoculated with PAN02 mouse pancreatic cancer cell line, grouped according to body weight and tumor volume on the 8th day after inoculation, and administered as described below.

Group 1 (control group): Vehicle control was administered intragastrically at a dose of 0.1 mL/10 g body weight twice a day.

Group 2: Compound 4 dissolved in (5% DMSO+40% PEG 400+10% Solutol+45% DDH₂O) was administered intragastrically at a dose of 30 mg/kg body weight.

During the assay, the body weight and tumor volume of mice were measured three times a week, and the tumor volume was calculated according to the formula of length×width²/2. The tumor proliferation rate and tumor inhibition rate were calculated according to the formula: tumor proliferation rate=tumor volume of treatment group/tumor volume of control group×100%; tumor inhibition rate=(tumor volume of control group−tumor volume of treatment group)/tumor volume of control group.

Student's t-test was used for statistical analysis between groups, and p<0.05 indicated significant difference.

Assay Results:

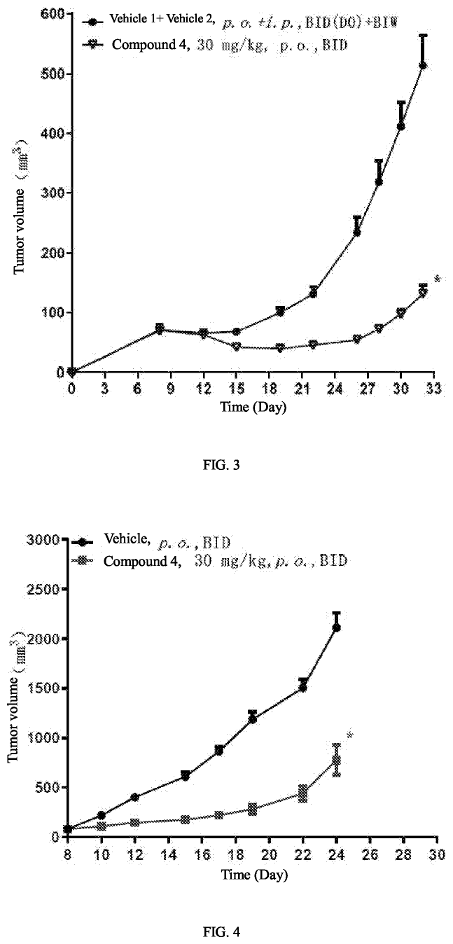

In the mouse PAN02 tumor model, Compound 4 showed anti-tumor activity at a dose of 30 mg/kg with a tumor proliferation rate of 37.61% on day 32. Compared with the control group, Compound 4 showed a significant anti-tumor effect (P=0.0417). The detailed results were shown in FIG. 3.

Assay Conclusion:

The compound of the present disclosure shows excellent anti-tumor effect in a PAN02 model.

### (3) Anti-Tumor Activity Assay of the Compound of the Present Disclosure in an EMT-6 In Vivo Tumor Model

Assay Purpose:

The anti-tumor effect of the test compound was investigated on an in vivo tumor model of mouse breast cancer EMT-6.

Assay Method:

Female BALB/C mice were subcutaneously inoculated with an EMT-6 mouse breast cancer cell line, grouped according to body weight and tumor volume on the 8th day after inoculation, and administered as described below.

Group 1 (control group): Vehicle control was administered intragastrically at a dose of 0.1 mL/10 g body weight twice a day.

Group 2: Compound 4 dissolved in (5% DMSO+40% PEG 400+10% Solutol+45% DDH₂O) was administered intragastrically at a dose of 30 mg/kg body weight.

During the assay, the body weight and tumor volume of mice were measured three times a week, and the tumor volume was calculated according to the formula of length×width²/2. The tumor proliferation rate and tumor inhibition rate were calculated according to the formula: tumor proliferation rate=tumor volume of treatment group/tumor volume of control group×100%; tumor inhibition rate=(tumor volume of control group-tumor volume of treatment group)/tumor volume of control group.

Student's t-test was used for statistical analysis between groups, and p<0.05 indicated significant difference.

Assay Results:

In the mouse EMT-6 tumor model, Compound 4 showed anti-tumor activity at a dose of 30 mg/kg with a tumor proliferation rate of 23.92% on Day 19. At the end of the assay, the tumor proliferation rate reached 36.79%. Compared with the control group, Compound 4 showed a significant anti-tumor effect (P=0.0493). The detailed results were shown in FIG. 4.

Assay Conclusion:

The compound of the present disclosure shows excellent anti-tumor effect in an EMT-6 model.

1. A compound represented by formula (I), or an isomer or a pharmaceutically acceptable salt thereof, 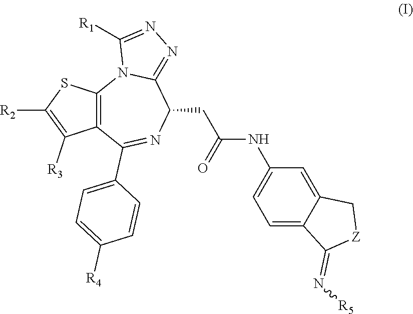
wherein,
R₁, R₂ and R₃ are each independently selected from C₁₋₃ alkyl optionally substituted with 1, 2 or 3 Rₐ;
R₄ is selected from the group consisting of H, F, Cl, Br, I, OH, and NH₂;
R₅ is selected from the group consisting of H, OH, NH₂, CN, C₁₋₆ alkyl, C₁₋₆ alkylamino, C₁₋₆ alkoxy and 4- to 6-membered heterocycloalkyl, wherein the C₁₋₆ alkyl, C₁₋₆ alkylamino, C₁₋₆ alkoxy and 4- to 6-membered heterocycloalkyl are each independently optionally substituted with 1, 2 or 3 R_{b};
Z is selected from the group consisting of O, NR₆ and CHR₆;
R₆ is selected from the group consisting of H and C₁₋₃ alkyl optionally substituted with 1, 2 or 3 R_{c};
or R₅, R₆ and the atoms to which they are attached together form 5- to 6-membered heterocycloalkenyl or 5- to 6-membered heteroaryl, wherein the 5- to 6-membered hetercyclooalkenyl and 5- to 6-membered heteroaryl are each independently optionally substituted with 1, 2 or 3 R_{d};
Rₐ, R_{c}, and R_{d} are each independently selected from the group consisting of F, Cl, Br, I, OH, NH₂, and CH₃;
R_{b} is selected from the group consisting of F, Cl, Br, I, OH, NH₂, CN, C₁₋₃ alkyl and C₁₋₃ alkoxy, wherein the C₁₋₃ alkyl and C₁₋₃ alkoxy are each independently optionally substituted with 1, 2 or 3 R;
R is each independently selected from the group consisting of F, Cl, Br, I, OH, NH₂ and CH₃;
the 4- to 6-membered heterocycloalkyl, 5- to 6-membered heterocycloalkenyl and 5- to 6-membered heteroaryl each contain 1, 2, 3 or 4 heteroatoms or heteroatom groups independently selected from the group consisting of —NH—, —O—, —S— and N.

2. The compound, or an isomer or a pharmaceutically acceptable salt thereof according to claim 1, wherein R₁, R₂ and R₃ are each independently selected from CH₃ optionally substituted with 1, 2 or 3 Rₐ.

3. The compound, or an isomer or a pharmaceutically acceptable salt thereof according to claim 2, wherein R₁, R₂ and R₃ are each independently selected from CH₃.

4. The compound, or an isomer or a pharmaceutically acceptable salt thereof according to claim 1, wherein R₄ is selected from CL.

5. The compound, or an isomer or a pharmaceutically acceptable salt thereof according to claim 1, wherein R_{b} is selected from the group consisting of F, Cl, Br, I, OH, NH₂, CN, CH₃, CH₂CH₃ and OCH₃, wherein the CH₃, CH₂CH₃ and OCH₃ are optionally substituted with 1, 2 or 3 R.

6. The compound, or an isomer or a pharmaceutically acceptable salt thereof according to claim 5, wherein R_{b} is selected from the group consisting of F, Cl, Br, I, OH, NH₂, CN, CH₃, CH₂F, CHF₂, CF₃, CH₂CH₃ and OCH₃.

7. The compound, or an isomer or a pharmaceutically acceptable salt thereof according to claim 1, wherein R₅ is selected from the group consisting of H, OH, NH₂, CN, C₁₋₃ alkyl, C₁₋₃ alkylamino, C₁₋₃ alkoxy and oxetanyl, wherein the C₁₋₃ alkyl, C₁₋₃ alkylamino, C₁₋₃ alkoxy or oxetanyl are each independently optionally substituted with 1, 2 or 3 R_{b}.

8. The compound, or an isomer or a pharmaceutically acceptable salt thereof according to claim 7, wherein R₅ is selected from the group consisting of H, OH, NH₂, CN, CH₃, CH₂CH₃, CH(CH₃)₂, OCH₃, OCH₂CH₃, NH(CH₃) and oxetanyl, wherein the CH₃, CH₂CH₃, CH(CH₃)₂, OCH₃, OCH₂CH₃, NH(CH₃) or oxetanyl are each independently optionally substituted with 1, 2 or 3 R_{b}.

9. The compound, or an isomer or a pharmaceutically acceptable salt thereof according to claim 6, wherein R₅ is selected from the group consisting of H, OH, NH₂, CN, CH₃, CH₂F, CHF₂, CF₃, CH₂CH₃, CH(CH₃)₂, OCH₃, OCH₂F, OCHF₂, OCF₃, OCH₂CH₃, OCH(CH₃)₂, OCH₂CH₂OCH₃, NH(CH₃), N(CH₃)₂, NHCH(CH₃)₂, and

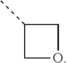

10. The compound, or an isomer or a pharmaceutically acceptable salt thereof according to claim 1, wherein R₅, R₆ and the atoms to which they are attached together form 4,5-dihydroisoxazolyl, pyrazolyl, pyrrolyl and imidazolyl, wherein the 4,5-dihydroisoxazolyl, pyrazolyl, pyrrolyl or imidazolyl are each independently optionally substituted with 1, 2 or 3 R_{d}.

11. The compound, or an isomer or a pharmaceutically acceptable salt thereof according to claim 10, wherein R₅, R₆ and the atoms to which they are attached together form

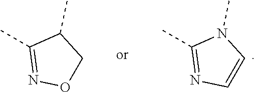

12. The compound, or an isomer or a pharmaceutically acceptable salt thereof according to claim 11, wherein the moiety 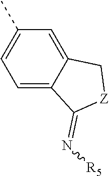
is selected from the group consisting of

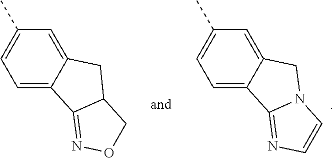

13. The compound, or an isomer or a pharmaceutically acceptable salt thereof according to claim 1, wherein the compound is selected from the group consisting of 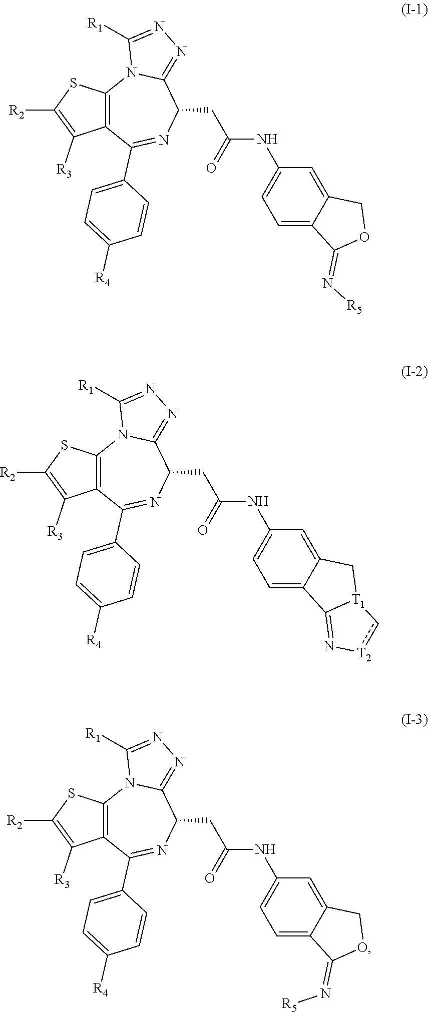
wherein, [symbol]
is selected from single bond and double bond;
R₁, R₂ and R₃ are as defined in any one of claims 1, 2 or 3;
R₄ is as defined in claim 1 or 4;
T₁ is CH or N;
T₂ is CH, N, or O;
R₅ is as defined in any one of claims 1, 7, 8 or 9.

14. The compound, or an isomer or a pharmaceutically acceptable salt thereof according to claim 13, wherein the compound is selected from the group consisting of 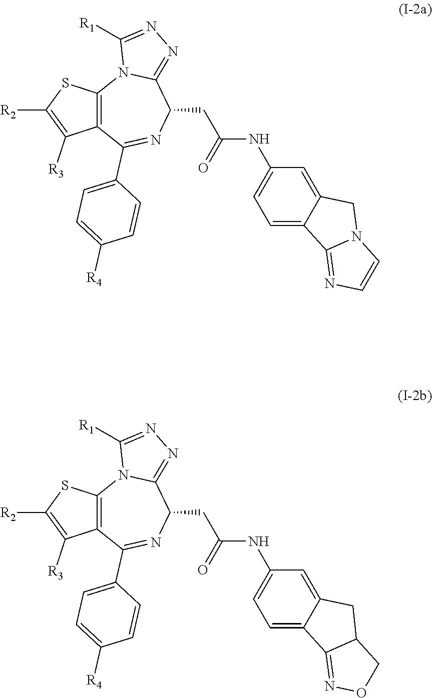
wherein,
R₁, R₂, R₃ and R₄ are as defined in claim 13.

15. A compound represented by the following formula, or an isomer or a pharmaceutically acceptable salt thereof, wherein the compound is selected from the group consisting of

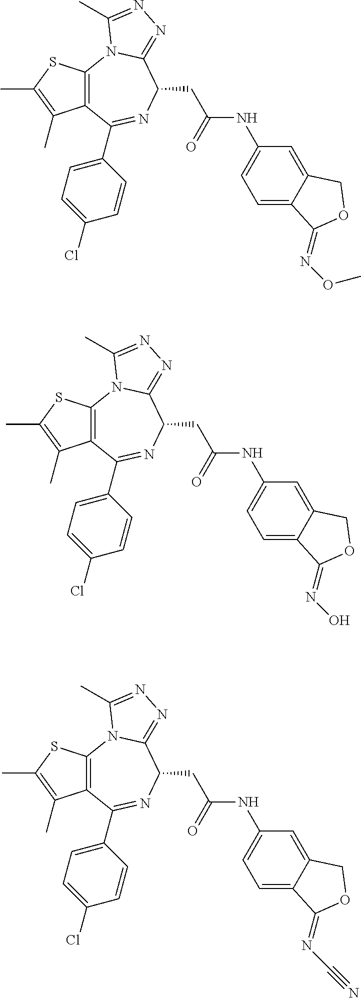

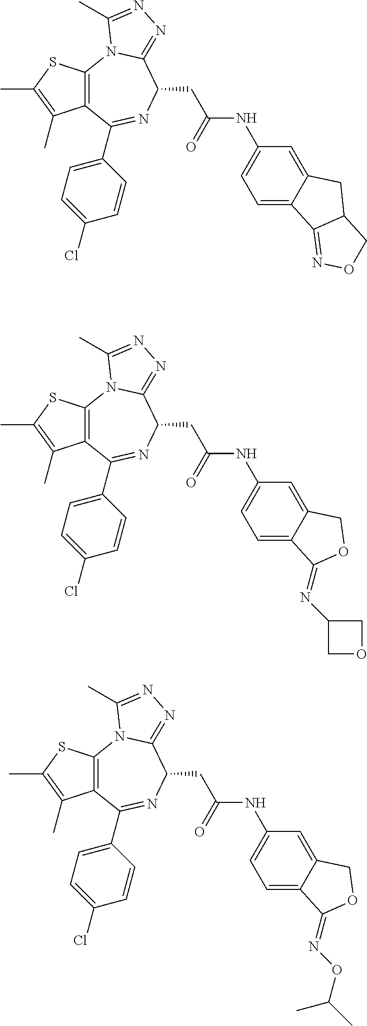 [SMILES: Cc1sc2c(c1C)C(c1ccc(Cl)cc1)=N[C@@H](CC(=O)Nc1ccc3c(c1)CC1CON=C31)c1nnc(C)n1-2 ; Cc1sc2c(c1C)C(c1ccc(Cl)cc1)=N[C@@H](CC(=O)Nc1ccc3c(c1)CO/C3=N\C1COC1)c1nnc(C)n1-2 ; Cc1sc2c(c1C)C(c1ccc(Cl)cc1)=N[C@@H](CC(=O)Nc1ccc3c(c1)CO/C3=N\OC(C)C)c1nnc(C)n1-2]

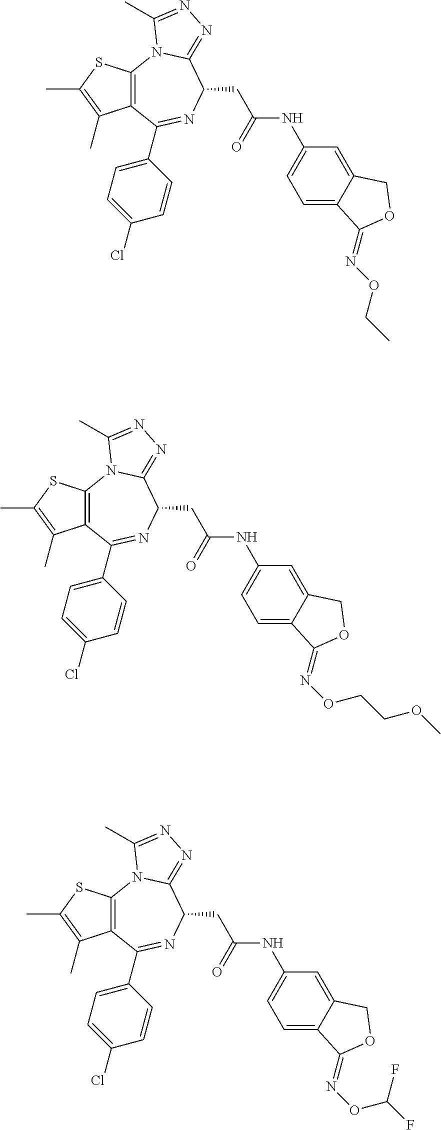

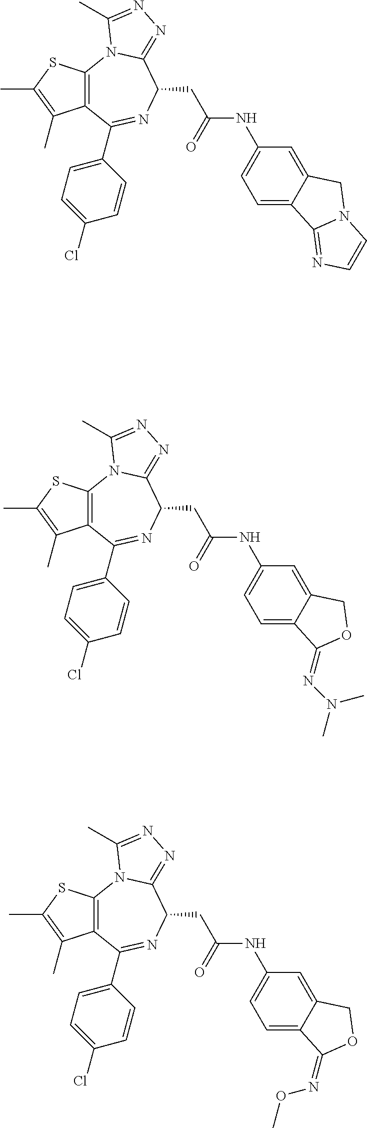

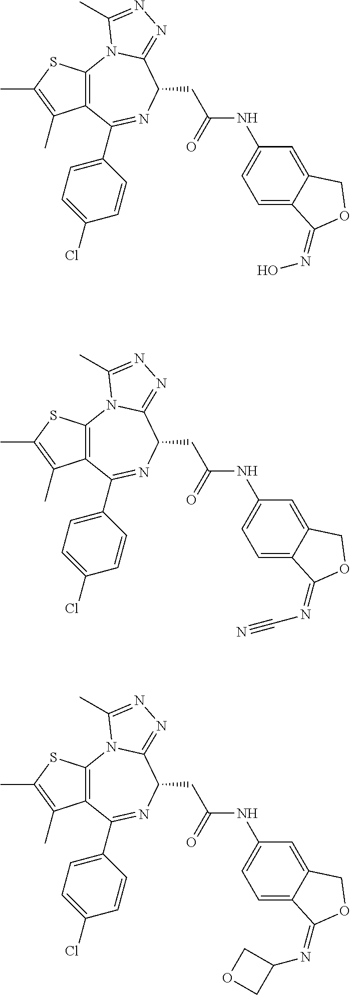 [SMILES: Cc1sc2c(c1C)C(c1ccc(Cl)cc1)=N[C@@H](CC(=O)Nc1ccc3c(c1)CO/C3=N/O)c1nnc(C)n1-2 ; Cc1sc2c(c1C)C(c1ccc(Cl)cc1)=N[C@@H](CC(=O)Nc1ccc3c(c1)CO/C3=N/C#N)c1nnc(C)n1-2 ; Cc1sc2c(c1C)C(c1ccc(Cl)cc1)=N[C@@H](CC(=O)Nc1ccc3c(c1)CO/C3=N/C1COC1)c1nnc(C)n1-2]

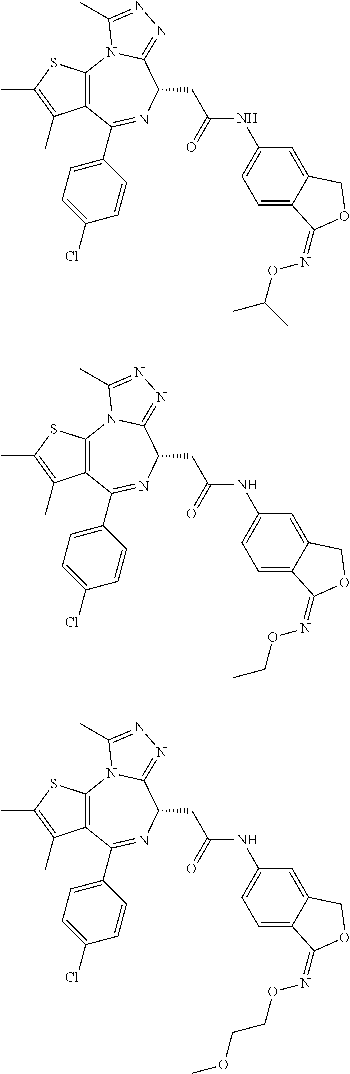 [SMILES: Cc1sc2c(c1C)C(c1ccc(Cl)cc1)=N[C@@H](CC(=O)Nc1ccc3c(c1)CO/C3=N/OC(C)C)c1nnc(C)n1-2 ; CCO/N=C1/OCc2cc(NC(=O)C[C@@H]3N=C(c4ccc(Cl)cc4)c4c(sc(C)c4C)-n4c(C)nnc43)ccc21 ; COCCO/N=C1/OCc2cc(NC(=O)C[C@@H]3N=C(c4ccc(Cl)cc4)c4c(sc(C)c4C)-n4c(C)nnc43)ccc21]

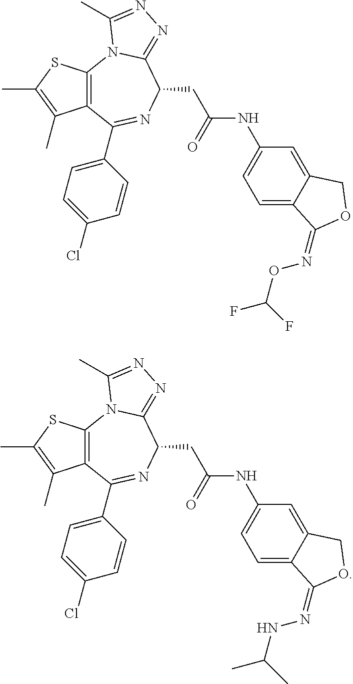 [SMILES: Cc1sc2c(c1C)C(c1ccc(Cl)cc1)=N[C@@H](CC(=O)Nc1ccc3c(c1)CO/C3=N/OC(F)F)c1nnc(C)n1-2 ; Cc1sc2c(c1C)C(c1ccc(Cl)cc1)=N[C@@H](CC(=O)Nc1ccc3c(c1)CO/C3=N/NC(C)C)c1nnc(C)n1-2]

16. The compound, or an isomer or a pharmaceutically acceptable salt thereof according to claim 15, wherein the compound is selected from the group consisting of

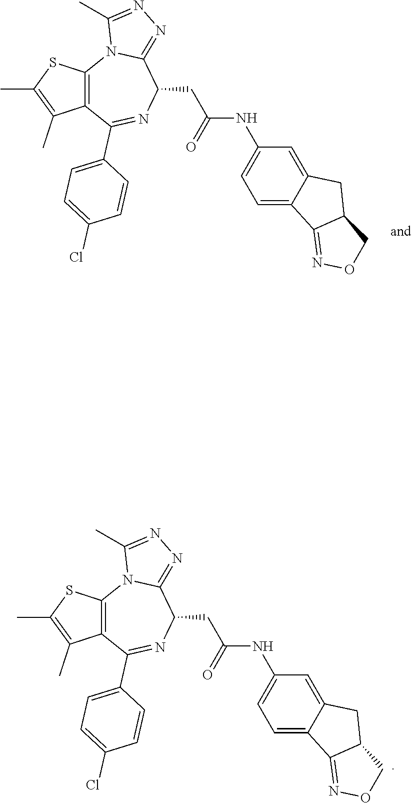 [SMILES: Cc1sc2c(c1C)C(c1ccc(Cl)cc1)=N[C@@H](CC(=O)Nc1ccc3c(c1)C[C@H]1CON=C31)c1nnc(C)n1-2 ; Cc1sc2c(c1C)C(c1ccc(Cl)cc1)=N[C@@H](CC(=O)Nc1ccc3c(c1)C[C@@H]1CON=C31)c1nnc(C)n1-2]

17. A pharmaceutical composition comprising a therapeutically effective amount of the compound, or an isomer or a pharmaceutically acceptable salt thereof according to claim 1 as an active ingredient and pharmaceutically acceptable carrier (s).

18. A method of treating a disease associated with BET Bromodomain protein inhibition and PD-L1 gene expression in a subject in need thereof, comprising administering to the subject the compound, or an isomer or a pharmaceutically acceptable salt thereof according to claim 1.

19. The method according to claim 18, wherein the disease is a tumor.

20. The compound, or an isomer or a pharmaceutically acceptable salt thereof according to claim 8, wherein R5 is selected from the group consisting of H, OH, NH2, CN, CH3, CH2F, CHF2, CF3, CH2CH3, CH(CH3)2, OCH3, OCH2F, OCHF2, OCF3, OCH2CH3, OCH(CH3)2, OCH2CH2OCH3, NH(CH3), N(CH3)2, NHCH(CH3)2, and [chemical structure]
